(19) United States
(12) Patent Application Publication
(10) Pub. No.: US 2008/0200471 A1
(43) Pub. Date: Aug. 21, 2008

(54) 6' SUBSTITUTED COMPOUNDS HAVING 5-HT6 RECEPTOR AFFINITY

(75) Inventors: Robert Dunn, Towaco, NJ (US); Truc Minh Nguyen, Des Moines, IA (US); Wenge Xie, Mahwah, NJ (US); Ashok Tehim, Ridgewood, NJ (US)

Correspondence Address: DARBY & DARBY P.C., P.O. BOX 770, Church Street Station, New York, NY, 10008-0770, US

(73) Assignee: MEMORY PHARMACEUTICALS CORPORATION, Montvale, NJ (US)

(21) Appl. No.: 12/033,797

(22) Filed: Feb. 19, 2008

Related U.S. Application Data
(60) Provisional application No. 60/890,324, filed on Feb. 16, 2007.

(51) Int. Cl. A61K 31/4985; C07D 295/00; A61P 25/00; A61P 1/00; A61K 31/496

(52) U.S. Cl. 514/249; 544/373; 514/254.09; 544/349; 544/371; 514/254.06

(57) ABSTRACT

The present disclosure provides compounds having affinity for the 5-HT6 receptor which are of the formula (I):

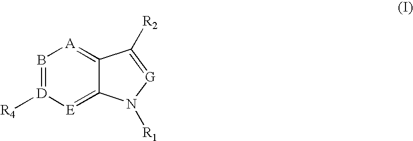

wherein R¹—R⁴ A, B, D, E, and G are as defined herein. The disclosure also relates to methods of preparing such compounds, compositions containing such compounds, and methods of use thereof.

This application claims priority to provisional application U.S. 60/890,324 filed Feb. 16, 2007, herein incorporated by reference in its entirety.

### FIELD OF THE INVENTION

The present invention relates generally to the field of serotonin 5-HT6 affinity. More specifically, this invention relates to novel compounds having affinity for the 5-HT6 receptor, in particular to compounds having selective 5-HT6 affinity, methods of preparing such compounds, compositions containing such compounds, and methods of use thereof.

### BACKGROUND OF THE INVENTION

The human 5-hydroxytryptamine-6 (5HT6) receptor, one of the most recently cloned serotonergic receptors, is a 440-amino acid polypeptide with seven transmembrane spanning domains typical of the G-protein-coupled receptors. It is one of the 14 receptors that mediate the effects of the neurotransmitter 5-hydroxytryptamine (5-HT, serotonin) (Hoyer et al., Neuropharmacology, 1997, 36:419). Within the transmembrane region, the human 5HT6 receptor shows about 30-40% homology to other human 5-HT receptors and is found to be positively coupled to adenylyl cyclase.

The prominent localization of 5HT6 receptor mRNA in the nucleus accumbens, striatum, olfactory tubercle, substantia nigra, and hippocampus of the brain (Ward et al., Neuroscience, 1995, 64:1105) together with its high affinity for several therapeutically important antipsychotics and antidepressants, suggest a possible role for this receptor in the treatment of schizophrenia and depression. In fact, the prototypic atypical antipsychotic agent clozapine exhibits greater affinity for the 5HT6 receptor than for any other receptor subtype (Monsma et al., J. Pharmacol. Exp. Ther., 1994, 268:1403).

Although the 5HT6 receptor has a distinct pharmacological profile, in vivo investigation of receptor function has been hindered by the lack of selective agonists and antagonists. Recent experiments demonstrated that chronic intracerebroventricular treatment with an antisense oligonucleotide, directed at 5HT6 receptor mRNA, elicited a behavioral syndrome in rats consisting of yawning, stretching, and chewing. This syndrome in the antisense-treated rats was dose-dependently antagonized by atropine (a muscarinic antagonist), implicating 5HT6 receptor in the control of cholinergic neurotransmission. Therefore, 5HT6 receptor antagonists may be useful for the treatment of memory dysfunction (Bourson et al., J. Pharmacol. Exp. Ther., 1995, 274:173), and to treat other central nervous system (CNS) disorders.

The high affinity of a number of antipsychotic agents for the 5-14T6 receptor, in addition to its mRNA localization in striatum, olfactory tubercle and nucleus accumbens suggests that some of the clinical actions of these compounds may be mediated through this receptor. Compounds which interact with, stimulate, or inhibit the 5-HT6 receptor are commonly referred to as 5-HT6 ligands. In particular, 5-HT6 selective ligands have been identified as potentially useful in the treatment of certain CNS disorders such as Parkinson's disease, Huntington's disease, anxiety, depression, manic depression, psychoses, epilepsy, obsessive compulsive disorders, migraine, Alzheimer's disease (enhancement of cognitive memory), sleep disorders, feeding disorders such as anorexia and bulimia, panic attacks, attention deficit hyperactivity disorder (ADHID), attention deficit disorder (ADD), withdrawal from drug abuse such as cocaine, ethanol, nicotine and benzodiazepines, schizophrenia, bipolar disorder, and also disorders associated with spinal trauma and/or head injury such as hydrocephalus. Such compounds are also expected to be of use in the treatment of certain gastrointestinal (GI) disorders such as functional bowel disorder and irritable bowel syndrome (See for ex. B. L. Roth et al., J. Pharmacol. Exp. Ther., 1994, 268, pages 1403-14120, D. R. Sibley et al., Mol. Pharmacol., 1993, 43, 320-327, A. J. Sleight et al., Neurotransmission, 1995, 11, 1-5, and A. J. Sleight et al. Serotonin ID Research Alert, 1997, 2 (3), 115-8). Furthermore, the effect of 5-HT6 antagonist and 5-HT6 antisense oligonucleotides to reduce food intake in rats has been reported (Br. J. Pharmac., 1999 Suppl. 126, page 66 and J. Psychopharmacol Suppl. A64, 1997, page 255).

Therefore, it is an object of this invention to provide compounds which are useful as therapeutic agents in the treatment of a variety of central nervous system disorders related to or affected by the 5-HT6 receptor.

It is another object of this invention to provide therapeutic methods and pharmaceutical compositions useful for the treatment of central nervous system disorders related to or affected by the 5-HT6 receptor.

The following patents and publications also provide relevant background to the present invention. All references cited below are incorporated herein by reference in their entirety and to the same extent as if each reference was individually incorporated by reference. U.S. Pat. Nos. 6,100,291, 6,133,287, 6,191,141, 6,251,893, 6,686,374, 6,767,912, 6,897,215, 6,903,112, 6,916,818, and 7,034,029; Published U.S. Application Nos. 2005/0124603, and 2005/0171118.

### SUMMARY OF THE INVENTION

The present invention relates to novel compounds that have affinity, preferably selectively, for the serotonin 5-H1T₆ receptor, methods of use thereof, and the synthesis thereof.

Still further, the present invention provides methods for synthesizing compounds with such activity and selectivity, as well as methods of and corresponding pharmaceutical compositions for treating a disorder (e.g. a mood disorder and/or a cognitive disorder) in a patient, wherein the disorder is related to or affected by the 5-HT6 receptor.

### DETAILED DESCRIPTION OF THE INVENTION

The present invention includes compounds of formula I:

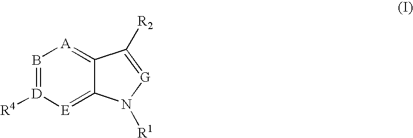

Wherein

A, B, E, and C; are each independently CH, CR³ or N;

D is C;

R¹ is SO₂Ar, wherein

Ar is selected from formulas (a)-(p):

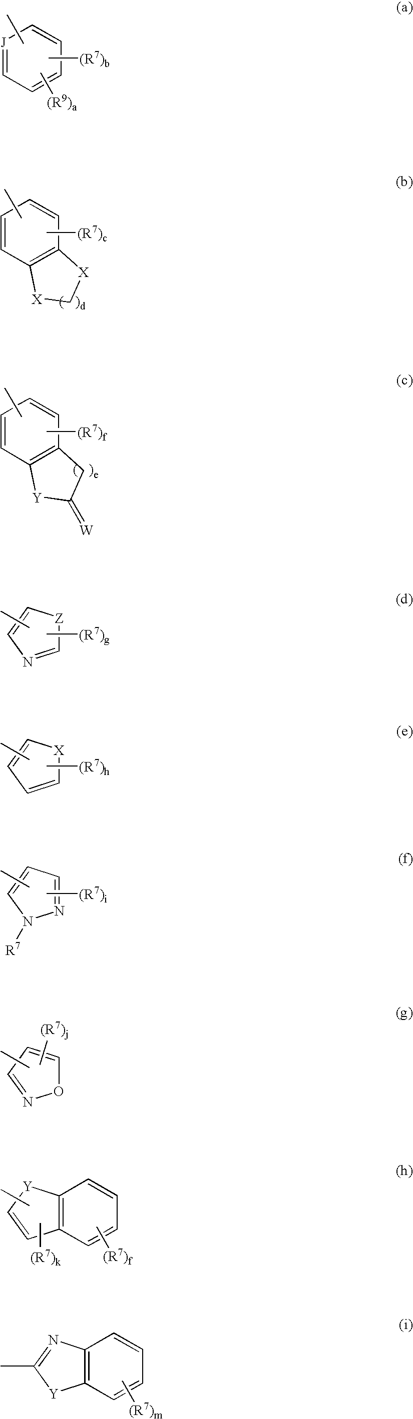

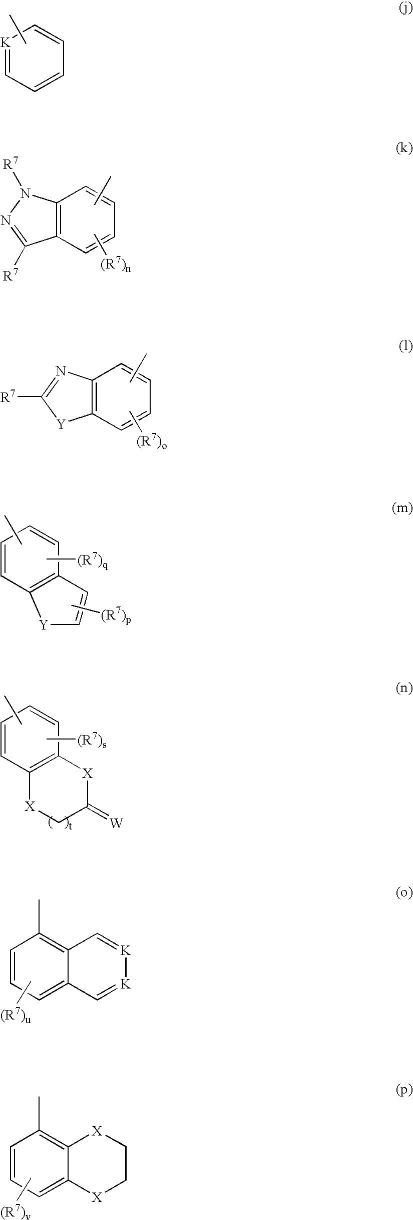

wherein

J is CR⁷ (e.g., CH) or N;

K is, in each instance is independently, CH or N;

W is O, S, or is absent;

X is, in each instance is independently, O or NR⁷;

Y is O, NR⁷ or S;

Z is S or NR⁷;

a is 1, 2, 3, 4 or 5;

b, l, and m are independently 0, 1, 2, 3 or 4;

c, f, h, n, o, q, s and u are independently 0, 1, 2 or 3;

d and e are independently 1, 2 or 3;

g, i, j, p, and u are independently 0, 1 or 2;

k and t are 0 or 1;

R² is H, C₁-C₆ alkyl, or COOR⁵

R³ is halogen (e.g., F), nitro,
alkyl having 1 to 8, preferably 1 to 4 carbon atoms, cycloalkyl having 3 to 12, preferably 3 to 8 carbon atoms, or cycloalkylalkyl having 4 to 12, preferably 4 to 8 carbon atoms, each of which is branched or unbranched and which is unsubstituted or substituted one or more times with halogen, C₁₋₄-alkyl, C₁₋₄-alkoxy, oxo, or any combination thereof (e.g., CHF₂, or CF₃), or
a heterocyclic group, which is saturated, partially saturated or unsaturated, having 5 to 10 ring atoms in which at least 1 ring atom is an N, O or S atom, which is unsubstituted or substituted one or more times by halogen, hydroxy, C₅₋₇-aryl, C₁₋₄-alkyl, C₁₋₄-alkoxy, cyano, halogenated C₁₋₄-alkyl (e.g., trifluoromethyl), nitro, or any combination thereof (e.g., substituted or unsubstituted morpholinyl, substituted or unsubstituted pyrrolyl, substituted or unsubstituted pyrrolidinyl, substituted or unsubstituted piperidinyl, substituted or unsubstituted pyridyl),
R⁴ is

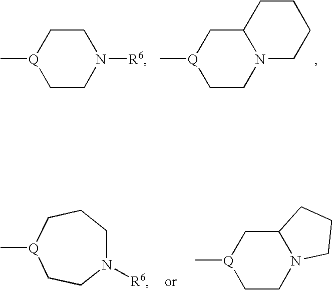

wherein each Q is independently N, CH, or C double bonded to an adjacent carbon,

R⁵ is H or alkyl having 1 to 8, preferably 1 to 4 carbon atoms (e.g., CH₃),

R⁶ is H or alkyl having 1 to 8, preferably 1 to 4 carbon atoms (e.g., CH₃), cycloalkyl having 3 to 12, preferably 3 to 8 carbon atoms, or cycloalkylalkyl having 4 to 12, preferably 4 to 8 carbon atoms, each of which is branched or unbranched and each of which is unsubstituted or substituted one or more times with halogen, C₁₋₄-alkyl, C₁₋₄-alkoxy, oxo, or any combination thereof;

R⁷ is, in each instance, independently
H, halogen (e.g., F, Cl, or Br), C(O)R⁸ (e.g., COCH₃), CO₂R⁸ (e.g., CO₂CH₃), NR⁶COR⁸ (e.g., NHCOCH₃),
alkyl having 1 to 12, preferably 1 to 8 carbon atoms, which is branched or unbranched and which is unsubstituted or substituted one or more times by halogen, hydroxy, cyano, C₁₋₄-alkoxy, oxo or any combination thereof (e.g., CH₃, CH₂CH₃, CHF₂, CF₃, etc.), and wherein optionally one or more —CH₂CH₂— groups is replaced in each case by —CH═CH— or —C≡C—,
alkoxy having 1 to 8, preferably 1 to 4 carbon atoms, which is branched or unbranched and which is unsubstituted or substituted one or more times by halogen (e.g., OCHF₂, or OCF₃),
cycloalkyl having 3 to 10, preferably 3 to 8 carbon atoms, which is unsubstituted or substituted one or more times by halogen, hydroxy, oxo, cyano, C₁₋₄-alkyl, C₁₋₄-alkoxy, or any combination thereof (e.g., cyclopentyl),
cycloalkylalkyl having 4 to 16, preferably 4 to 12 carbon atoms, which is unsubstituted or substituted in the cycloalkyl portion and/or the alkyl portion one or more times by halogen, oxo, cyano, hydroxy, C₁₋₄-alkyl, C₁₋₄-alkoxy or any combination thereof (e.g., cyclopentylmethyl or cyclopropylmethyl,),
aryl having 6 to 14 carbon atoms, which is unsubstituted or substituted one or more times by halogen, CF₃, OCF₃, C₁₋₄-alkyl, hydroxy, C₁₋₄-alkoxy, nitro, methylenedioxy, ethylenedioxy, cyano, or any combination thereof (e.g., substituted or unsubstituted phenyl, or substituted or unsubstituted pyridinyl),
arylalkyl in which the aryl portion has 6 to 14 carbon atoms and the alkyl portion, which is branched or unbranched, has 1 to 5 carbon atoms, wherein the arylalkyl radical is unsubstituted, substituted in the aryl portion one or more times by halogen, CF₃, OCF₃, C₁₋₄-alkyl, hydroxy, C₁₋₄-alkoxy, nitro, cyano, methylenedioxy, ethylenedioxy, or any combination thereof, and/or substituted in the alkyl portion one or more times by halogen, oxo, hydroxy, cyano, or any combination thereof, and wherein in the alkyl portion one or more —CH₂CH₂— groups are each optionally replaced by —CH═CH— or —C/C—, and one or more —CH₂— groups are each optionally replaced by —O— or —NH— (e.g., phenylethyl, phenylpropyl, phenylbutyl, methoxyphenylethyl, methoxyphenylpropyl, chlorophenylethyl, chlorophenylpropyl, phenylethenyl, phenoxyethyl, phenoxybutyl, chlorophenoxyethyl, or chlorophenylaminoethyl),
a heterocyclic group, which is saturated, partially saturated or unsaturated, having 5 to 10 ring atoms in which at least 1 ring atom is an N, O or S atom, which is unsubstituted or substituted one or more times by halogen, hydroxy, C₅₋₇-aryl, C₁₋₄-alkyl, C₁₋₄-alkoxy, cyano, trifluoromethyl, nitro, oxo, or any combination thereof (e.g., substituted or unsubstituted morpholinyl), or
a heterocycle-alkyl group, wherein the heterocyclic portion is saturated, partially saturated or unsaturated, and has 5 to 10 ring atoms in which at least 1 ring atom is an N, O or S atom, and the alkyl portion is branched or unbranched and has 1 to 5 carbon atoms, the heterocycle-alkyl group is unsubstituted, substituted one or more times in the heterocyclic portion by halogen, OCF₃, hydroxy, C₅₋₇-aryl, C₁₋₄-alkyl, C₁₋₄-alkoxy, cyano, trifluoromethyl, nitro, oxo, or any combination thereof, and/or substituted in the alkyl portion one or more times by halogen, oxo, hydroxy, cyano, or any combination thereof, and wherein in the alkyl portion one or more —CH₂CH₂— groups are each optionally replaced by —CH═CH— or —C/C—, and one or more —CH₂— groups are each optionally replaced by —O— or —NH—;

R⁸ is in each instance, independently, H or alkyl having 1 to 8, carbon atoms, preferably 1 to 4 carbon atoms, which is branched or unbranched and which is unsubstituted or substituted one or more times by halogen (e.g., CH₃, CH₂CH₃, CHF₂, or CF₃);

R⁹ is amino NH₂), C₁₋₄-alkylamino, C₁₋₄-dialkylamino (e.g., NMe₂), NR¹⁰C(O)R¹⁰ (e.g., —NHC(O)CH₃ , —N(CH₃)C(O)CH₃)), cyano, methoxy, or a helerocyclic group, which is saturated, partially saturated or unsaturated, having 5 to 10 ring atoms in which at least 1 ring atom is an N, O or S atom, which is unsubstituted or substituted one or more times by halogen, hydroxy, C₅₋₇-aryl, C₁₋₄-alkyl, C₁₋₄-alkoxy, cyano, halogenated C₁₋₄-alkyl (e.g., trifluoromethyl), nitro, or any combination thereof (e.g., substituted or unsubstituted morpholinyl, substituted or unsubstituted pyrimidinyl, or substituted or unsubstituted pyrrolidinyl), or —C(O)-heterocyclic group,

R¹⁰ in each instance, is independently H or alkyl having 1 to 8, preferably 1 to 4 carbon atoms, which is branched or unbranched and which is unsubstituted or substituted one or more times with halogen, C₁₋₄-alkyl, C₁₋₄-alkoxy, oxo, or any combination thereof;

and pharmaceutically acceptable salts or solvates (e.g., hydrates) thereof, or solvates of pharmaceutically acceptable salts thereof;

with the following provisos:
(i) wherein if A, B, A, and G are CH or CR³ D is C, and Ar is then R⁴ is not

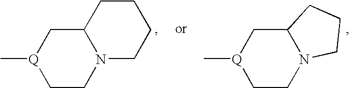

(ii) wherein if A, B, and E are CH or CR³, D is C, G is N, R² is H, and Ar is (j) wherein K is CH, or (h) wherein Y is S, then R⁴ is not

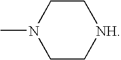

Halogen herein refers to F, Cl, Br, and I. Preferred halogens are F and Cl.

Alkyl means a straight-chain or branched-chain aliphatic hydrocarbon radical. Suitable alkyl groups include, but are not limited to, methyl, ethyl, propyl, isopropyl, butyl, sec-butyl, tert-butyl, pentyl, hexyl, heptyl, octyl, nonyl, decyl, undecyl, and dodecyl. Other examples of suitable alkyl groups include, but are not limited to, 1-, 2- or 3-methylbutyl, 1,1-, 1,2- or 2,2-dimethylpropyl, 1-ethylpropyl, 1-, 2, -, 3- or 4-methylpentyl, 1,1-, 1,2-, 1,3-, 2,2-, 2,3- or 3,3-dimethylbutyl, 1- or 2-ethylbutyl, ethylmethylpropyl, trimethylpropyl, methylhexyl, dimethylpentyl, ethylpentyl, ethylmethylbutyl, dimethylbutyl, and the like.

These alkyl radicals can optionally have one or more —CH₂CH₂— groups replaced in each case by —CH═CH— or —C≡C— groups. Suitable alkenyl or alkynyl groups include, but are not limited to, 1-propenyl, 2-propenyl, 1-propynyl, 1-butenyl, 2-butenyl, 3-butenyl, 1-butynyl, 1,3-butadienyl, and 3-methyl-2-butenyl.

The alkyl groups include cycloalkyl groups, e.g., monocyclic, bicyclic or tricyclic saturated hydrocarbon radical having 3 to 8 carbon atoms, preferably 3 to 6 carbon atoms. Suitable cycloalkyl groups include, but are not limited to, cyclopropyl, cyclobutyl, cyclopentyl, cyclohexyl, cycloheptyl, cyclooctyl, and norbornyl. Other suitable cycloalkyl groups include, but are not limited to, spiropentyl, bicyclo[2.1.0]pentyl, bicyclo[3.1.0]hexyl, spiro[2.4]heptyl, spiro[2.5]octyl, bicyclo[5.1.0]octyl, spiro[2.6]nonyl, bicyclo[2.2.0]hexyl, spiro[3.3]heptyl, and bicyclo[4.2.0]octyl.

The alkyl groups also include cycloalkylalkyl in which the cycloalkyl portions have preferably 3 to 8 carbon atoms, preferably 4 to 6 carbon atoms and alkyl the portions have preferably 1 to 8 carbon atoms, preferably 1 to 4 carbon atoms. Suitable examples include, but are not limited to, cyclopentylethyl and cyclopropylmethyl.

In the arylalkyl groups and heteroalkyl groups, “alkyl” refers to a divalent alkylene group preferably having 1 to 4 carbon atoms.

In the cases where alkyl is a substituent (e.g., alkyl substituents on aryl and heteroaryl groups) or is part of a substituent (e.g., in the alkylamino, dialkylamino, hydroxyalkyl, hydroxyalkoxy, alkylthio, alkylsulphinyl, and alkylsulphonyl substituents), the alkyl portion preferably has 1 to 12 carbon atoms, especially 1 to 8 carbon atoms, in particular 1 to 4 carbon atoms.

Aryl, as a group or substituent per se or as part of a group or substituent, refers to an aromatic carbocyclic radical containing 6 to 14 carbon atoms, preferably 6 to 12 carbon atoms, especially 6 to 10 carbon atoms. Suitable aryl groups include, but are not limited to, phenyl, naphthyl and biphenyl. Substituted aryl groups include the above-described aryl groups which are substituted one or more times by, for example, halogen, alkyl, hydroxy, alkoxy, nitro, methylenedioxy, ethylenedioxy, amino, alkylamino, dialkylamino, hydroxyalkyl, hydroxyalkoxy, carboxy, cyano, acyl, alkoxycarbonyl, alkylthio, alkylsulphinyl, alkylsulphonyl, phenoxy, and acyloxy (e.g., acetoxy).

Arylalkyl refers to an aryl-alkyl-radical in which the aryl and alkyl portions are in accordance with the previous descriptions. Suitable examples include, but are not limited to, benzyl, 1-phenethyl, 2-phenethyl, phenpropyl, phenbutyl, phenpentyl, and naphthalenemethyl.

Heteroaryl groups refer to unsaturated heterocyclic groups having one or two rings and a total number of 5 to 10 ring atoms wherein at least one of the ring atoms is preferably an N, O or S atom. Preferably, the heteroaryl group contains 1 to 3, especially 1 or 2, hetero-ring atoms selected from N, O and S. Suitable heteroaryl groups include, for example, furyl, benzothienyl, benzofuranyl, pyrrolyl, pyrazolyl, imidazolyl, pyridyl, pyrimidinyl, isoxazolyl, quinolinyl, azaindolyl, naphthyridinyl, thiazolyl, and the like. Preferred heteroaryl groups include, but are not limited to, furyl, benzothienyl, benzofuranyl, pyrrolyl, pyrazolyl, imidazolyl, pyridyl, pyrimidinyl, isoxazolyl, and thiazolyl.

Substituted heteroaryl groups refer to the heteroaryl groups described above which are substituted in one or more places by preferably halogen, aryl, alkyl, alkoxy, cyano, halogenated alkyl (e.g., trifluoromethyl), nitro, oxo, amino, alkylamino, and dialkylamino.

Hetereocycles are non-aromatic, saturated or partially unsaturated, cyclic groups containing at least one hetero-ring atom, preferably selected from N, S, and O, for example, 1,2,3,4,-tetrahydroquinolyl, dihydrobenzofuranyl, dihydrobenzodioxepinyl, dihydrobenzodioxinyl, dihydroindolyl, benzodioxolyl, 3-tetrahydrofuranyl, piperidinyl, imidazolinyl, imidazolidinyl, pyrrolinyl, pyrrolidinyl, morpholinyl, piperazinyl, oxazolidinyl, and indolinyl.

Heteroarylalkyl refers to a heteroaryl-alkyl-group wherein the heteroaryl and alkyl portions are in accordance with the previous discussions. Suitable examples include, but are not limited to, pyridylmethyl, thienylmethyl, pyrimidinylmethyl, pyrazinylmethyl, isoquinolinylmethyl, pyridylethyl and thienylethyl.

Carbocyclic structures are non-aromatic monocyclic or bicyclic structures containing 5 to 14 carbon atoms, preferably 6 to 10 carbon atoms, wherein the ring structure(s) optionally contain at least one C═C bond.

Acyl refers to alkanoyl radicals having 2 to 4 carbon atoms. Suitable acyl groups include, but are not limited to, formyl, acetyl, propionyl, and butanoyl.

Substituted radicals preferably have 1 to 3 substituents, especially 1 or 2 substituents.

R² is preferably H; an alkyl having 1 to 4 carbon atoms, e.g., methyl, ethyl, propyl, isopropyl, n-butyl, especially methyl or ethyl; or a carboxyl group, e.g., carboxylic acid, methyl carboxylate, ethyl carboxylate or propyl carboxylate.

R³ is preferably H or alkyl having 1 to 4 carbon atoms, e.g., methyl, ethyl, propyl, isopropyl, n-butyl, especially methyl. More preferably, R³ is H.

R4 is a preferably

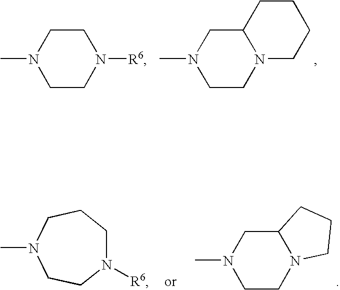

In another preferred embodiment, R⁴ is

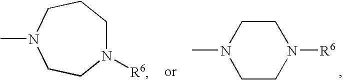

In one embodiment, R⁴ is preferably

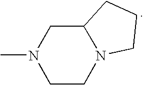

In one preferred embodiment, R⁴ is

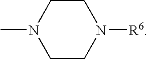

In another preferred embodiment, R⁶ is an alkyl having 1 to 8, preferably 1 to 4 carbon atoms.

R⁵ is preferably methyl or ethyl.

R⁶ is preferably H or methyl.

R⁷ is preferably C₁₋₄-alkyl (e.g., methyl, ethyl), halogenated C₁₋₄-alkyl (e.g., CH₂, CF₃), aryl (e.g., unsubstituted or substituted phenyl), CO₂R₈ (e.g., CO₂CH₃), NR⁶COR₈ (e.g., NHCOCH3, N(CH₃)COCH₃), halogen (e.g., F, Cl), or C(O)R⁸ (e.g., COCH₃). In a preferred embodiment, R⁷ is a C₁₋₄ alkyl or C(O)CH₃.

R⁸ is preferably alkyl having 1 to 4 carbon atoms, e.g., CH₃, CH₂CH₃, especially CH₃.

In one preferred embodiment, R⁹ is preferably amino (NH₂), C₁₋₄-alkylamino, C₁₋₄-dialkylamino (e.g., NMe₂), NR¹⁰OC(O)R¹⁰ (e.g., —NHC(O)CH₃, or —N(CH₃)C(O)CH₃)) or a heterocyclic group.

In one embodiment R⁹ is preferably a heterocycle and more preferably a pyrrolidine or a substituted pyrrolidine, e.g., methoxy pyrrolidine or pyrrolidinol.

In another embodiment, R⁹ is preferably a pyrrolidine or a substituted pyrrolidine, e.g., methoxy pyrrolidine or pyrrolidinol.

Y is preferably O or NR⁷.

W is preferably absent, or when present, is preferably O.

In a preferred embodiment, Ar is selected from formulas (a), (b), (c′), (j), (m), (n), and (p):

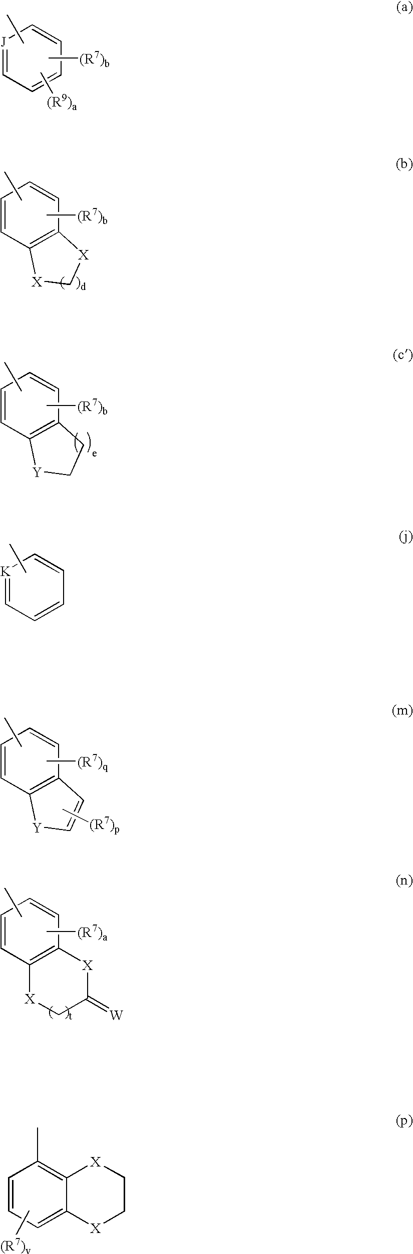

In a preferred embodiment, Ar is selected from formulas (a), (b), (c′), (j), (m), and (n). In another preferred embodiment Ar is selected from formulas (a), (b), (c′), (m), (n), and (p).

In another preferred embodiment, Ar is selected from the formula (b), (c), (f), (g), (i), (k), (l), (n), (o), and (p).

In another preferred embodiment, Ar is (b), d is 2, one X is O and the second X is NR7.

In another preferred embodiment, Ar is (c), e is 1 and W is absent. In a particularly preferred embodiment, Y is NR7.

In another preferred embodiment, Ar is (n), t is 1 and W is present.

In yet another preferred embodiment, Ar is (o) and at least one K is NR7.

Preferred examples of Ar represented by formulas (a)-(p) include, but are not limited to, phenyl substituted at least once by amino, dialkylamino (e.g. N(CH₃)₂), NR COR (e.g., NHCOCH₃), N(CH₃)COCH₃), or substituted or unsubstituted heterocyclic group (e.g., pyrimidinyl, pyrrolidinyl, morpholinyl); pyridinyl substituted at least once by substituted or unsubstituted heterocyclic group (e.g., morpholinyl); unsubstituted or substituted dihydrobenzofuranyl (e.g., 2,3-dihydrobenzofuran-5-yl); unsubstituted or substituted dihydrobenzodioxepinyl (e.g., 3,4-dihydro-2H-1,5-benzodioxepin-7-yl); unsubstituted or substituted thiazolyl (e.g., 4-alkyl-2-aryl-substituted thiazolyl); unsubstituted or substituted pyrazolyl (e.g., 5-methyl-1-phenyl-1H-pyrazol-4-yl, 1-methyl-3-trifluoromethyl-1H-pyrazol-4-yl, 1,3,5-trimethyl-1-H-pyrazol-4-yl, 1-ethyl-3-methyl-1H-pyrazol-4-yl, 1-difluoromethyl-5-methyl-1H-pyrazol-4-yl, 1-difluoromethyl-3-methyl-1H-pyrazol-4-yl, 1-ethyl-5-methyl-1H-pyrazol-4-yl, 1-ethyl-1H-pyrazol-4-yl, 1-ethyl-3,5-dimethyl-1H-pyrazol-4-yl, 1-methyl-1H-pyrazol-4-yl, 1,5-dimethyl-1H-pyrazol-4-yl); unsubstituted or substituted benzothienyl (e.g., 1-benzothien-2-yl, 1-benzothien-3-yl); unsubstituted or substituted furanyl (e.g., 5-acetoxy-furan-2-yl, 2,5-dimethyl-furan-3-yl); unsubstituted or substituted benzofuranyl (e.g., 1-benzofuran-2-yl); unsubstituted or substituted oxazolyl (e.g., 3,5-dimethyloxazol-4-yl); unsubstituted or substituted benzothiazolyl (e.g., 1,3-benzothiazol-6-yl); unsubstituted or substituted pyrrolyl (e.g., 4-chloro-1,2-dimethyl-1-H-pyrrol-3-yl); unsubstituted or substituted imidazolyl (e.g., 1-methyl-1H-imidazol-4-yl, 1,2-dimethyl-1H-imidazol-4-yl); unsubstituted or substituted dihydroindolyl (e.g., 2,3,dihydro-1-H-indol-5-yl, 1-acetyl-2,3,dihydro-1-H-indol-5-yl, 1-methyl-2,3,dihydro-1-H-indol-5-yl, 1-ethyl-2,3,dihydro-1-H-indol-5-yl); unsubstituted or substituted indazolyl (e.g., 1-(2,2-dimethylpropanoyl)indazol-5-yl); and unsubstituted or substituted tetrahydroisoquinolinyl (e.g., 1,2,3,4-tetrahydroisoquinolin-7-yl, 1-methyl-1,2,3,4-tetrahydroisoquinolin-7-yl, 1 -methyl-1,2,3,4-tetrahydroisoquinolin-7-yl).

In addition, preferred compounds in accordance with the invention are described by subformulas (i)-(v), which correspond to formula I, but exhibit the following preferred groups:
(i) A and B are CH,
D is C,
E and G are CH or N,

R¹ is SO₂Ar wherein Ar is phenyl substituted at least once by 3-methoxypyrrolidinyl, 3-hydroxypyrrolidinyl, or pyrrolidin-3-ol, or Ar is a substituted or unsubstituted aryl selected from pyrrolo[2,3-b]pyridinyl, benzofuranyl, dihydroindolyl, piperazinyl-indazolyl, and pyrazolo[3,4-b]pyridinyl.
(ii) A and B are CH, D is CR,

R¹ is SO₂Ar wherein Ar is an unsubstituted phenyl or unsubstituted pyridyl, and
R⁴ is

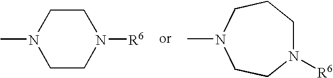

wherein R₆ is H or methyl.
(iii) at least on of A, B, and E is CR³ or N.
(iv) R⁴ is

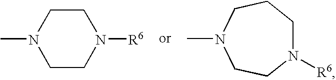

(v) G is CH or CR⁴.

One aspect of the present invention comprises compounds 1-94 or a(nother) salt or freebase thereof. Another aspect of the present invention comprises compounds 1-96 or a(nother) salt or freebase thereof. Another aspect of the present invention comprises compounds 1-94 and 104-115 or a(nother) salt or freebase thereof. Another aspect of the present invention comprises compounds I -115 and 140-141 or a(nother) salt or freebase thereof. Another aspect of the present invention comprises compounds 1-137 and 140-141 or a(nother) salt or freebase thereof. Another aspect of the present invention comprises compounds 1-94, 104-115 and 140-142 or a(nother) salt or freebase thereof.

According to a compound and/or method aspect of the present invention, the compounds are selected from:

| No. | Name |
|---|---|
| 1 | 4-methyl-7-[(6-piperazin-1-yl-1H-indol-1-yl)sulfonyl]- |
| | 3,4-dihydro-2H-1,4-benzoxazine |
| 2 | 1-methyl-5-[(6-piperazin-1-yl-1H-indol-1-yl)sulfonyl]-1H-indole |
| 3 | 1-[(1-acetyl-2,3-dihydro-1H-indol-5-yl)sulfonyl]- |
| | 6-piperazin-1-yl-1H-indole |
| 4 | 1-{[3-(3-methoxypyrrolidin-1-yl)phenyl]sulfonyl}-6- |
| | (4-methylpiperazin-1-yl)-1H-pyrazolo[3,4-b]pyridine |
| 5 | 1-{[3-(3-methoxypyrrolidin-1-yl)phenyl]sulfonyl}-6- |
| | piperazin-1-yl-1H-pyrazolo[3,4-b]pyridine |
| 6 | 7-[(6-piperazin-1-yl-1H-pyrazolo[3,4-b]pyridin-1-yl) |
| | sulfonyl]-2H-1,4-benzoxazin-3(4H)-one |
| 7 | 1-{[3-(3-methoxypyrrolidin-1-yl)phenyl]sulfonyl}- |
| | 6-(4-methylpiperazin-1-yl)-1H-indazole |
| 8 | 7-{[6-(4-methylpiperazin-1-yl)-1H-pyrazolo[3,4-b] |
| | pyridin-1-yl]sulfonyl}-2H-1,4-benzoxazin-3(4H)-one |
| 9 | 7-{[6-(4-methylpiperazin-1-yl)-1H-indazol-1-yl] |
| | sulfonyl}-2H-1,4-benzoxazin-3(4H)-one |
| 10 | 7-{[6-(4-methylpiperazin-1-yl)-1H-indol-1-yl] |
| | sulfonyl}-2H-1,4-benzoxazin-3(4H)-one |
| 11 | 7-[(6-piperazin-1-yl-1H-indol-1-yl)sulfonyl]- |
| | 2H-1,4-benzoxazin-3(4H)-one |
| 12 | 1-{[3-(3-methoxypyrrolidin-1-yl)phenyl]sulfonyl}- |
| | 6-(4-methylpiperazin-1-yl)-1H-indole |
| 13 | 1-{[3-(3-methoxypyrrolidin-1-yl)phenyl]sulfonyl}- |
| | 6-piperazin-1-yl-1H-indole |
| 14 | 1-[(1-acetyl-2,3-dihydro-1H-indol-5-yl)sulfonyl]-6- |
| | (4-methylpiperazin-1-yl)-1H-pyrrolo[2,3-b]pyridine |
| 15 | 2-{1-[(1-acetyl-2,3-dihydro-1H-indol-5-yl)sulfonyl]- |
| | 1H-indol-6-yl}octahydro-2H-pyrido[1,2-a]pyrazine |
| 16 | 2-{1-[(1-acetyl-2,3-dihydro-1H-indol-5-yl)sulfonyl]- |
| | 1H-indol-6-yl}octahydropyrrolo[1,2-a]pyrazine |
| 17 | 1-{[3-(3-methoxypyrrolidin-1-yl)phenyl]sulfonyl}- |
| | 6-(4-methylpiperazin-1-yl)-1H-pyrrolo[2,3-b]pyridine |
| 18 | 2-(1-{]3-(3-methoxypyrrolidin-1-yl)phenyl]sulfonyl}- |
| | 1H-indol-6-yl)octahydro-2H-pyrido[1,2-a]pyrazine |
| 19 | 2-(1-{]3-(3-methoxypyrrolidin-1-yl)phenyl]sulfonyl}- |
| | 1H-indol-6-yl)octahydropyrrolo[1,2-a]pyrazine |
| 20 | 1-[(1-acetyl-2,3-dihydro-1H-indol-5-yl)sulfonyl]- |
| | 6-(4-methylpiperazin-1-yl)-1H-pyrazolo[3,4-b]pyridine |
| 21 | 1-[(1-acetyl-2,3-dihydro-1H-indol-5-yl)sulfonyl]- |
| | 6-(4-methylpiperazin-1-yl)-1H-indazole |
| 22 | 1-[(1-acetyl-2,3-dihydro-1H-indol-5-yl)sulfonyl]-6- |
| | (4-methylpiperazin-1-yl)-1H-indole |
| 23 | 4-methyl-7-{[6-(4-methylpiperazin-1-yl)-1H-indazol- |
| | 1-yl]sulfonyl}-3,4-dihydro-2H-1,4-benzoxazine |
| 24 | 4-methyl-7-{[6-(4-methylpiperazin-1-yl)-1H-pyrazolo |
| | [3,4-b]pyridin-1-yl]sulfonyl}-3,4-dihydro- 2H-1,4-benzoxazine |
| 25 | 4-methyl-7-{[6-(4-methylpiperazin-1-yl)-1H-pyrrolo[2,3-b] |
| | pyridin-1-yl]sulfonyl}-3,4-dihydro- 2H-1,4-benzoxazine |
| 26 | 4-methyl-7-{[6-(octahydro-2H-pyrido[1,2-a]pyrazin-2-yl)- |
| | 1H-indol-1-yl]sulfonyl}-3,4-dihydro-2H-1,4-benzoxazine |
| 27 | 7-{[6-(hexahydropyrrolo[1,2-a]pyrazin-2(1H)-yl)-1H- |
| | indol-1-yl]sulfonyl}-4-methyl-3,4-dihydro-2H-1,4-benzoxazine |
| 28 | 7-{[6-(4-methylpiperazin-1-yl)-1H-pyrrolo[2,3-b]pyridin-1-yl] |
| | sulfonyl}-2H-1,4-benzoxazin-3(4H)-one |
| 29 | 7-{[6-(octahydro-2H-pyrido[1,2-a]pyrazin-2-yl)-1H-indol-1-yl] |
| | sulfonyl}-2H-1,4-benzoxazin-3(4H)-one |
| 30 | 7-{[6-(hexahydropyrrolo[1,2-a]pyrazin-2(1H)-yl)-1H-indol-l-yl] |
| | sulfonyl}-2H-1,4-benzoxazin-3(4H)-one |
| 31 | 1-(1-benzofuran-5-ylsulfonyl)-6-(4-methylpiperazin-1-yl)-1H- |
| | pyrrolo[2,3-b]pyridine |
| 32 | 2-[1-(1-benzofuran-5-ylsulfonyl)-1H-indol-6-yl]octahydro-2H- |
| | pyrido[1,2-a]pyrazine |
| 33 | 1-[(1-acetyl-2,3-dihydro-1H-indol-5-yl)sulfonyl]-6- |
| | (1,4-diazepan-1-yl)-1H-indazole |
| 34 | 7-{[6-(1,4-diazepan-1-yl)-1H-indazol-1-yl]sulfonyl}-4- |
| | methyl-3,4-dihydro-2H-1,4-benzoxazine |
| 35 | 1-(1-benzofuran-6-ylsulfonyl)-6-(1,4-diazepan-1-yl)-1H-indazole |
| 36 | 1-(1-benzofuran-5-ylsulfonyl)-6-(1,4-diazepan-1-yl)-1H-indazole |
| 37 | 1-[(1-acetyl-2,3-dihydro-1H-indol-5-yl)sulfonyl]-6- |
| | (4-methyl-1,4-diazepan-1-yl)-1H-indole |
| 38 | 4-methyl-7-{[6-(4-methyl-1,4-diazepan-1-yl)-1H-indol-1-yl] |
| | sulfonyl}-3,4-dihydro-2H-1,4-benzoxazine |
| 39 | 1-{[3-(3-methoxypyrrolidin-1-yl)phenyl] |
| | sulfonyl}-6-(4-methyl-1,4-diazepan-1-yl)-1H-indole |
| 40 | 1-[(1-acetyl-2,3-dihydro-1H-indol-5-yl)sulfonyl]-6- |
| | (1,4-diazepan-1-yl)-1H-indole |
| 41 | 7-{[6-(1,4-diazepan-1-yl)-1H-indol-1-yl]sulfonyl}-4- |
| | methyl-3,4-dihydro-2H-1,4-benzoxazine |
| 42 | 6-(1,4-diazepan-1-yl)-1-{[3- |
| | (3-methoxypyrrolidin-1-yl)phenyl]sulfonyl }-1H-indole |
| 43 | 1-(1-benzofuran-6-ylsulfonyl)-6-(1,4-diazepan-1-yl)-1H-indole |
| 44 | 1-(1-benzofuran-5-ylsulfonyl)-6-(1,4-diazepan-1-yl)-1H-indole |
| 45 | 6-(1,4-diazepan-1-yl)-1-{[3-(3-methoxypyrrolidin-1-yl) |
| | phenyl]sulfonyl}-1H-indazole |
| 46 | 4-methyl-7-{[6-(4-methyl-1,4-diazepan-1-yl)-1H-indazol-1-yl] |
| | sulfonyl}-3,4-dihydro-2H-1,4-benzoxazine |
| 47 | 1-{[3-(3-methoxypyrrolidin-1-yl)phenyl]sulfonyl}-6- |
| | (4-methyl-1,4-diazepan-1-yl)-1H-indazole |
| 48 | 1-(1-benzofuran-6-ylsulfonyl)-6-(4-methyl-1,4-diazepan-1-yl)- |
| | 1H-indazole |
| 49 | 1-(1-benzofuran-5-ylsulfonyl)-6-(4-methyl-1,4-diazepan-1-yl)- |
| | 1H-indazole |
| 50 | 1-[(1 acetyl-2,3-dihydro-1H-indol-5-yl)sulfonyl]-6- |
| | piperazin-1-yl-1H-indole |
| 51 | 4-methyl-7-{[6-(4-methylpiperazin-1-yl)-1H-indazol- |
| | 1-yl]sulfonyl}-3,4-dihydro-2H-1,4-benzoxazine |
| 52 | ethyl 1-[(4-methyl-3,4-dihydro-2H-1,4-benzoxazin-7-yl)sulfonyl]- |
| | 6-(4-methylpiperazin-1-yl)-1H-indazole-3-carboxylate |
| 53 | ethyl 1-{[3-(3-hydroxypyrrolidin-1-yl)phenyl]sulfonyl}- |
| | 6-(4-methylpiperazin-1-yl)-1H-indazole-3-carboxylate |
| 54 | ethyl 1 -[(4-methyl-3,4-dihydro-2H-1,4-benzoxazin-7-yl)sulfonyl]- |
| | 6-piperazin-1-yl-1H-indazole-3-carboxylate |
| 55 | ethyl 1-{[3-(3-methoxypyrrolidin-1-yl)phenyl]sulfonyl}- |
| | 6-piperazin-1-yl-1H-indazole-3-carboxylate |
| 56 | 7-{[3-ethyl-6-(4-methylpiperazin-1-yl)-1H-indazol-1-yl] |
| | sulfonyl}-4-methyl-3,4-dihydro-2H-1,4-benzoxazine |
| 57 | 3-ethyl-1-{[3-(3-methoxypyrrolidin-1-yl)phenyl]sulfonyl}- |
| | 6-(4-methylpiperazin-1-yl)-1H-indazole |
| 58 | 1-(3-{[3-ethyl-6-(4-methylpiperazin-1-yl)-1H-indazol-1-yl] |
| | sulfonyl}phenyl)pyrrolidin-3-ol |
| 59 | 7-[(3-ethyl-6-piperazin-1-yl-1H-indazol-1-yl)sulfonyl]- |
| | 4-methyl-3,4-dihydro-2H-1,4-benzoxazine |
| 60 | 3-ethyl-1-{[3-(3-methoxypyrrolidin-1-yl)phenyl] |
| | sulfonyl}-6-piperazin-1-yl-1H-indazole |
| 61 | 1-{3-[(3-ethyl-6-piperazin-1-yl-1H-indazol-1-yl) |
| | sulfonyl]phenyl}pyrrolidin-3-ol |
| 62 | 1-[(1-acetyl-2,3-dihydro-1H-indol-5-yl)sulfonyl]- |
| | 3-ethyl-6-piperazin-1-yl-1H-indazole |
| 63 | ethyl 1-{[3-(3-methoxypyrrolidin-1-yl)phenyl]sulfonyl}- |
| | 6-(4-methylpiperazin-1-yl)-1H-indazole-3-carboxylate |
| 64 | 7-[(3 -ethyl-6-piperazin-1-yl-1H-indazol-1-yl)sulfonyl]-2H- |
| | 1,4-benzoxazin-3(4H)-one |
| 65 | 7-[(6-piperazin-1-yl-1H-indol-1-yl)sulfonyl]- |
| | 2H-1,4-benzoxazin-3(4H)-one |
| 66 | 6-[(3-ethyl-6-piperazin-1-yl-1H-indol-1-yl)sulfonyl]- |
| | 4-methyl-3,4-dihydro-2H-1,4-benzoxazine |
| 67 | 6-[(3-ethyl-6-piperazin-1-yl-1H-indol-1-yl) |
| | sulfonyl]-2H-1,4-benzoxazin-3(4H)-one |
| 68 | 3-Ethyl-1-[3-((S)-3-methoxy-pyrrolidin-1-yl)-benzenesulfonyl]-6- |
| | piperazin-1-yl-1H-indole |
| 69 | (S)-1-[3-(3-Ethyl-6-piperazin-1-yl-indole-1-sulfonyl)- |
| | phenyl]-pyrrolidin-3-ol |
| 70 | 5-(6-Piperazin-1-yl-indole-1-sulfonyl)-4H-benzol[1,4]oxazin-3-one |
| 71 | 4-methyl-7-[(6-piperazin-1-yl-1H-indazol-1-yl)sulfonyl]- |
| | 3,4-dihydro-2H-1,4-benzoxazine |
| 72 | 4-methyl-7-[(3-methyl-6-piperazin-1-yl-1H-indazol-1-yl)sulfonyl]- |
| | 3,4-dihydro-2H-1,4-benzoxazine |
| 73 | 4-methyl-7-[(3-methyl-6-piperazin-1-yl-1H-indol-1-yl)sulfonyl]- |
| | 3,4-dihydro-2H-1,4-benzoxazine |
| 74 | 7-[(3-ethyl-6-piperazin-1-yl-1H-indol-1-yl)sulfonyl]-4-methyl- |
| | 3,4-dihydro-2H-1,4-benzoxazine |
| 75 | 4-methyl-6-[(6-piperazin-1-yl-1H-indazol-1-yl)sulfonyl]-3,4- |
| | dihydro-2H-1,4-benzoxazine |
| 76 | 4-methyl-6-[(6-piperazin-1-yl-1H-indol-1-yl)sulfonyl]- |
| | 3,4-dihydro-2H-1,4-benzoxazine |
| 77 | 4-Methyl-6-(3-methyl-6-piperazin-1-yl-indazole-1- |
| | sulfonyl)-3,4-dihydro-2H-benzo[1,4]oxazine |
| 78 | 3-Ethyl-1-[3-((S)-3-methoxy-pyrrolidin-1-yl)-benzenesulfonyl]- |
| | 6-piperazin-1-yl-1H-indazole |
| 79 | 3-Ethyl-1-[3-((S)-3-methoxy-pyrrolidin-1-yl)-benzenesulfonyl]- |
| | 6-piperazin-1-yl-1H-indazole |
| 80 | 1-[3-((S)-3-Methoxy-pyrrolidin-1-yl)-benzenesulfonyl]- |
| | 3-methyl-6-piperazin-1-yl-1H-indazole |
| 81 | 1-[3-((S)-3-Methoxy-pyrrolidin-1-yl)-benzenesulfonyl]- |
| | 3-methyl-6-piperazin-1-yl-1H-indole |
| 82 | 4-Methyl-6-(3-methyl-6-piperazin-1-yl-indole-1-sulfonyl)- |
| | 3,4-dihydro-2H-benzo[1,4]oxazine |
| 83 | 6-(3-Ethyl-6-piperazin-1-yl-indole-1-sulfonyl)-4-methyl-3,4- |
| | dihydro-2H-benzo[1,4]oxazine |
| 84 | 7-(3-Ethyl-6-piperazin-1-yl-indazole-1-sulfonyl)-4-methyl-3,4- |
| | dihydro-2H-benzo[1,4]oxazine |
| 85 | 6-(6-Piperazin-1-yl-indazole-1-sulfonyl)-4H- |
| | benzo[1,4]oxazin-3-one |
| 86 | 6-(6-Piperazin-1-yl-indole-1-sulfonyl)-4H-benzo[1,4]oxazin-3-one |
| 87 | 6-(3-Methyl-6-piperazin-1-yl-indazole-1-sulfonyl)-4H-benzo |
| | [1,4]oxazin-3-one |
| 88 | 6-(3-Methyl-6-piperazin-1-yl-indole-1-sulfonyl)-4H-benzo |
| | [1,4]oxazin-3-one |
| 89 | 4-methyl-7-[(6-piperazin-1-yl-1H-indazol-1-yl)sulfonyl]- |
| | 3,4-dihydro-2H-pyrido[3,2-b][1,4]oxazine |
| 90 | 1-(2,3-dihydro-1,4-benzodioxin-6-ylsulfonyl)-6-piperazin- |
| | 1-yl-1H-indole |
| 91 | {3-[(6-piperazin-1-yl-1H-indazol-1-yl)sulfonyl] |
| | phenyl}(pyridin-2-yl)methanone |
| 92 | 1-(2,3-dihydro-1,4-benzodioxin-6-ylsulfonyl)-6- |
| | piperazin-1-yl-1H-indazole |
| 93 | {3-[(6-piperazin-1-yl-1H-indol-1-yl) |
| | sulfonyl]phenyl }(pyridin-2-yl)methanone |
| 94 | 3-[(6-piperazin-1-yl-1H-indazol-1-yl)sulfonyl]benzonitrile |
| 95 | 3-[(6-piperazin-1-yl-1H-indol-1-yl)sulfonyl]benzonitrile |
| 96 | 6-(4-methylpiperazin-1-yl)-1-(phenylsulfonyl)-1H-indole |
| 97 | 1-(phenylsulfonyl)-6-piperazin-1-yl-1H-indole |
| 98 | ethyl 6-(4-methylpiperazin-1-yl)-1-(phenylsulfonyl)- |
| | 1H-indazole-3-carboxylate |
| 99 | 6-(4-methylpiperazin-1-yl)-1-(phenylsulfonyl)-1H- |
| | indazole-3-carboxylic acid |
| 100 | ethyl 1-(phenylsulfonyl)-6-piperazin-1-yl-1H-indazole- |
| | 3-carboxylate |
| 101 | 3-ethyl-6-(4-methylpiperazin-1-yl)-1-(phenylsulfonyl)- |
| | 1H-indazole |
| 102 | 3-ethyl-1-(phenylsulfonyl)-6-piperazin-1-yl-1H-indazole |
| 103 | ethyl 6-(4-methylpiperazin-1-yl)-1-(phenylsulfonyl)-1H- |
| | indazole-3-carboxylate |
| 104 | 6-piperazin-1-yl-1-(pyridin-3-ylsulfonyl)-1H-indole |
| 105 | 6-(4-methylpiperazin-1-yl)-1-(pyridin-3-ylsulfonyl)-1H-indole |
| 106 | 6-(4-methylpiperazin-1-yl)-1-(pyridin-3-ylsulfonyl)-1H-indazole |
| 107 | 6-piperazin-1-yl-1-(pyridin-3-ylsulfonyl)-1H-indole |
| 108 | 6-(4-methylpiperazin-1-yl)-1-(pyridin-3-ylsulfonyl)- |
| | 1H-pyrrolo[2,3-b]pyridine |
| 109 | 6-(4-methyl-1,4-diazepan-1-yl)-1-(pyridin-3-ylsulfonyl)-1H-indole |
| 110 | 6-(1,4-diazepan-1-yl)-1-(pyridin-3-ylsulfonyl)-1H-indole |
| 111 | 6-(1,4-diazepan-1-yl)-1-(pyridin-3-ylsulfonyl)-1H-indazole |
| 112 | ethyl 6-piperazin-1-yl-1-(pyridin-3-ylsulfonyl)-1H- |
| | indazole-3-carboxylate |
| 113 | 3-ethyl-6-(4-methylpiperazin-1-yl)-1-(pyridin-3-ylsulfonyl)- |
| | 1H-indazole |
| 114 | 3-ethyl-6-piperazin-1-yl-1-(pyridin-3-ylsulfonyl)-1H-indazole |
| 115 | 6-piperazin-1-yl-1-(pyridin-3-ylsulfonyl)-1H-indole |
| 116 | 1-[(3-fluorophenyl)sulfonyl]-6-piperazin-1-yl-1H-indole |
| 117 | 1-[(2-fluorophenyl)sulfonyl]-6-piperazin-1-yl-1H-indole |
| 118 | 1-[(2,4-difluorophenyl)sulfonyl]-6-piperazin-1-yl-1H-indole |
| 119 | 1-[(2,5-difluorophenyl)sulfonyl]-6-piperazin-1-yl-1H-indole |
| 120 | 1-[(3-chlorophenyl)sulfonyl]-6-piperazin-1-yl-1H-indole |
| 121 | 1-[(2-chlorophenyl)sulfonyl]-6-piperazin-1-yl-1H-indole |
| 122 | 1-[(3-fluorophenyl)sulfonyl]-6-piperazin-1-yl-1H-indazole |
| 123 | 1-[(2-fluorophenyl)sulfonyl]-6-piperazin-1-yl-1H-indazole |
| 124 | 1-[(2,4-difluorophenyl)sulfonyl]-6-piperazin-1-yl-1H-indazole |
| 125 | 1-[(2,5-difluorophenyl)sulfonyl]-6-piperazin-1-yl-1H-indazole |
| 126 | 1-[(3-chlorophenyl)sulfonyl]-6-piperazin-1-yl-1H-indazole |
| 127 | 1-[(2-chlorophenyl)sulfonyl]-6-piperazin-1-yl-1H-indazole |
| 128 | 1-[(3-methoxyphenyl)sulfonyl]-6-piperazin-1-yl-1H-indole |
| 129 | 1-[(2-methoxyphenyl)sulfonyl]-6-piperazin-1-yl-1H-indole |
| 130 | -[(4-methoxyphenyl)sulfonyl]-6-piperazin-1-yl-1H-indole |
| 131 | 1-[(3,4-dimethoxyphenyl)sulfonyl]-6-piperazin-1-yl-1H-indole |
| 132 | 1-[(2,5-dimethoxyphenyl)sulfonyl]-6-piperazin-1-yl-1H-indole |
| 133 | 1-[(3-methoxyphenyl)sulfonyl]-6-piperazin-1-yl-1H-indazole |
| 134 | 1-[(2-methoxyphenyl)sulfonyl]-6-piperazin-1-yl-1H-indazole |
| 135 | 1-[(4-methoxyphenyl)sulfonyl]-6-piperazin-1-yl-1H-indazole |
| 136 | 1-[(3,4-dimethoxyphenyl)sulfonyl]-6-piperazin-1-yl-1H-indazole |
| 137 | 1-[(2,5-dimethoxyphenyl)sulfonyl]-6-piperazin-1-yl-1H-indazole |
| 138 | 1-(1-naphthylsulfonyl)-6-piperazin-1-yl-1H-indazole |
| 139 | 1-(1-naphthylsulfonyl)-6-piperazin-1-yl-1H-indole |
| 140 | 3-[(6-piperazin-1-yl-1H-indol-1-yl)sulfonyl]quinoline |
| 141 | 3-[(6-piperazin-1-yl-1H-indazol-1-yl)sulfonyl]quinoline |

wherein salts listed above can also be in free base form or in the form of another pharmaceutically acceptable salt, and free base forms listed above can also be in the form of a pharmaceutically acceptable salt,

wherein a compound listed above (either in a free base form or in the form of a pharmaceutically acceptable salt) can also be in the form of a solvate (such as a hydrate),

wherein a compound listed above (in a free base form or solvate thereof, or in the form of a pharmaceutically acceptable salt or solvate thereof) can also be in the form of a polymorph, and

wherein if the compound exhibits chirality it can be in the form of a mixture of enantiomers such as a racemate or a mixture of diastereomers, or can be in the form of a single enantiomer or a single diastereomer.

The following table presents structures for selected compounds of the present invention:

| | Structure |
|---|---|
| 1 | 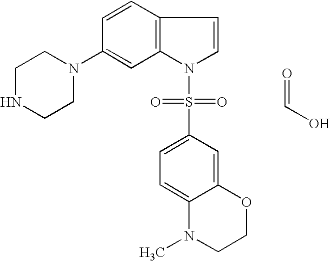 |
| 2 | 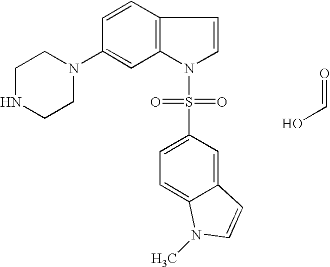 |
| 3 | 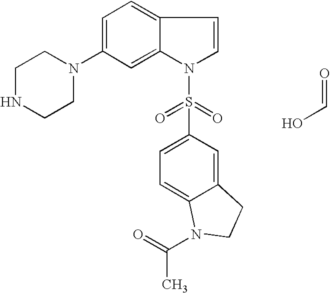 |
| 4 | 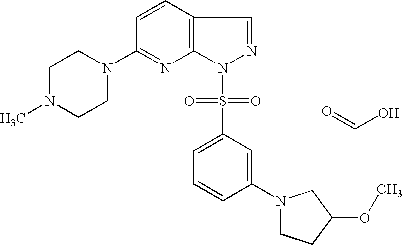 |
| 5 | 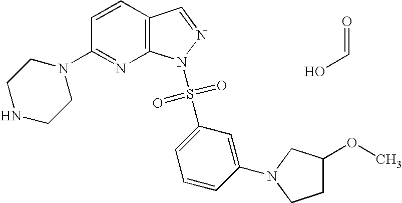 |
| 6 | 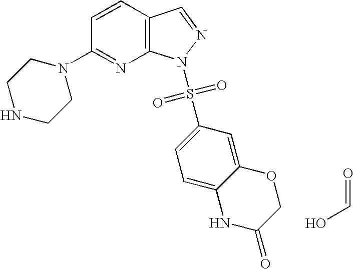 |
| 7 | 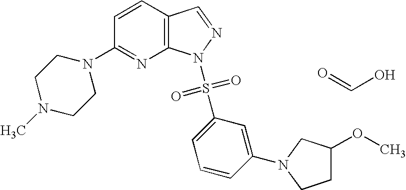 |
| 8 | 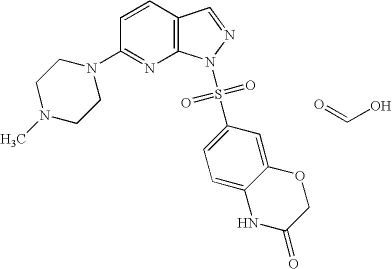 |
| 9 | 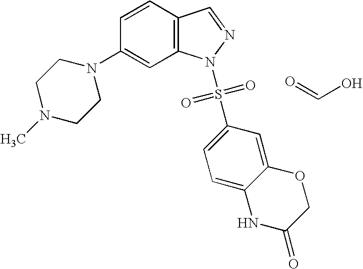 |
| 10 | 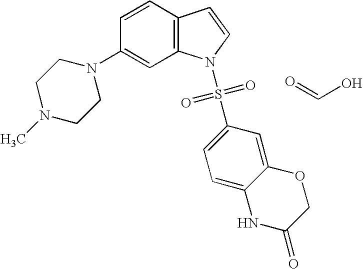 |
| 11 | 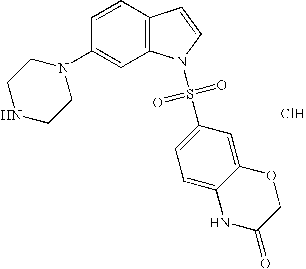 |
| 12 | 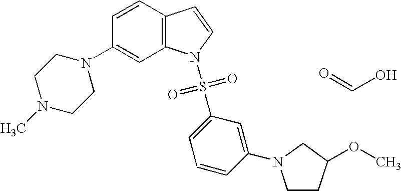 |
| 13 | 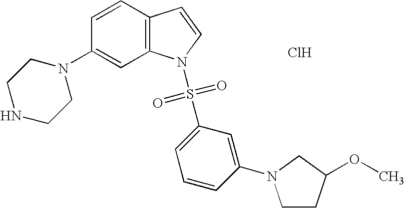 |
| 14 | 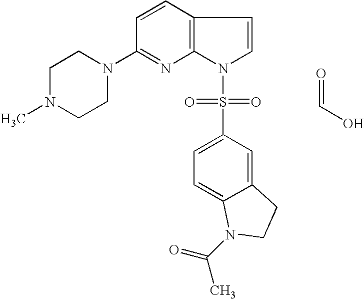 |
| 15 | 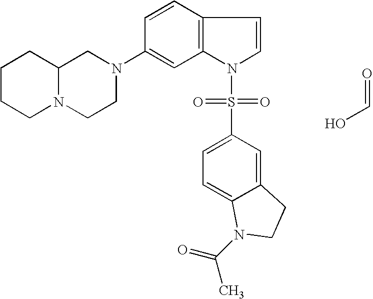 |
| 16 | 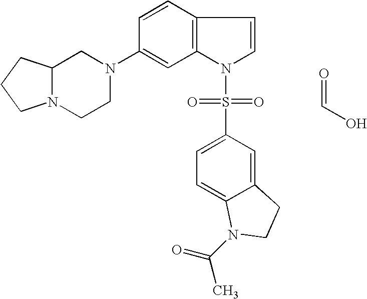 |
| 17 | 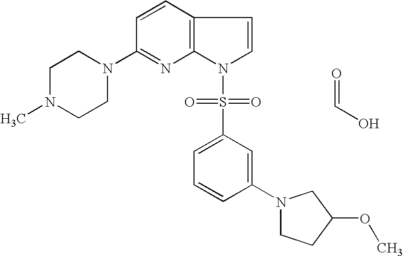 |
| 18 | 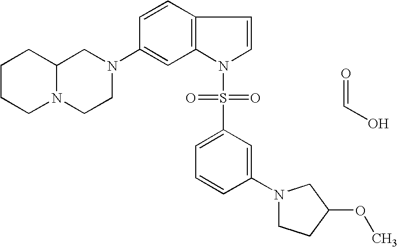 |
| 19 | 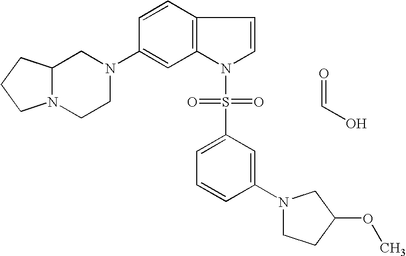 |
| 20 | 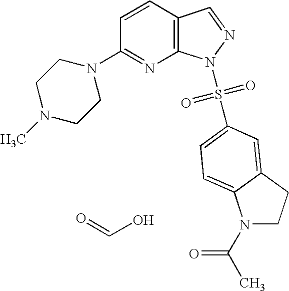 |
| 21 | 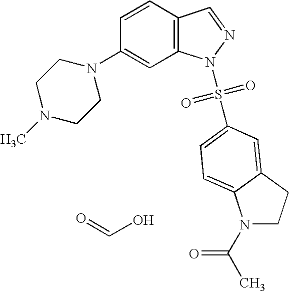 |
| 22 | 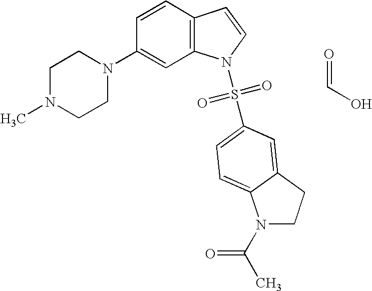 |
| 23 | 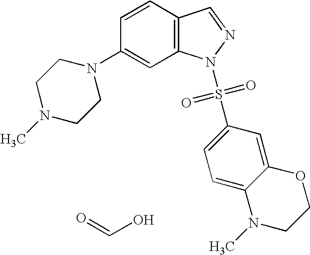 |
| 24 | 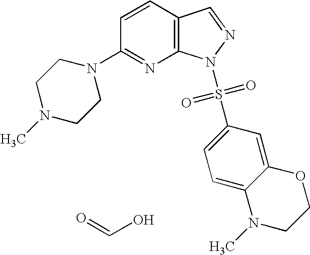 |
| 25 | 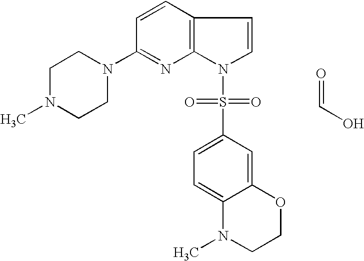 |
| 26 | 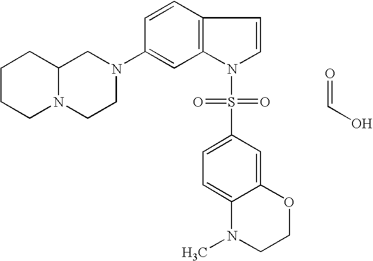 |
| 27 | 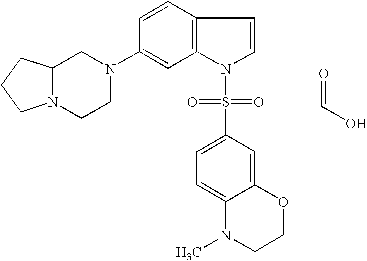 |
| 28 | 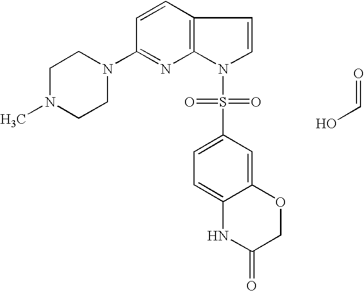 |
| 29 | 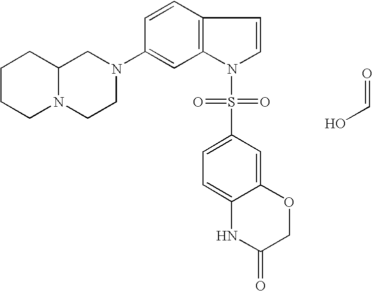 |
| 30 | 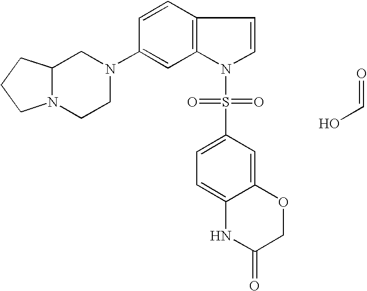 |
| 31 | 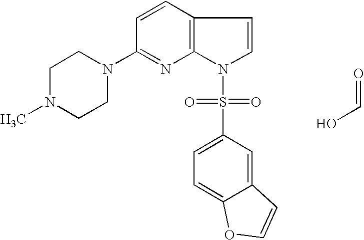 |
| 32 | 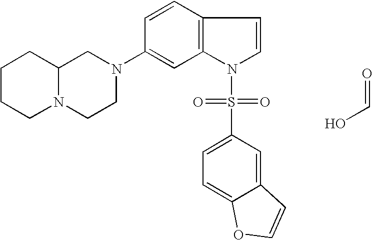 |
| 33 | 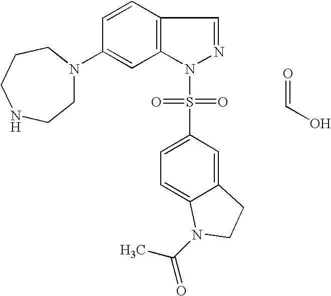 |
| 34 | 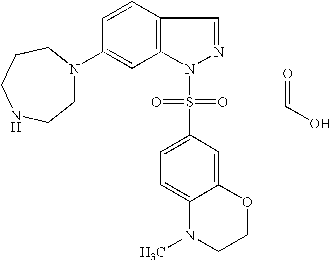 |
| 35 | 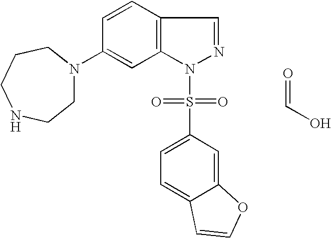 |
| 36 | 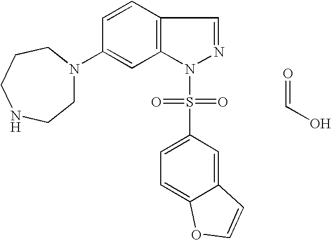 |
| 37 | 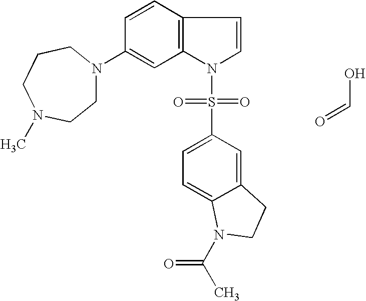 |
| 38 | 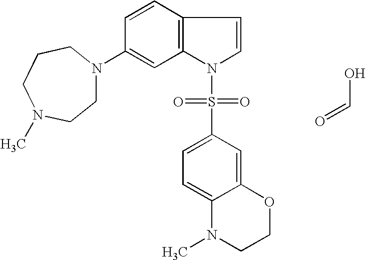 |
| 39 | 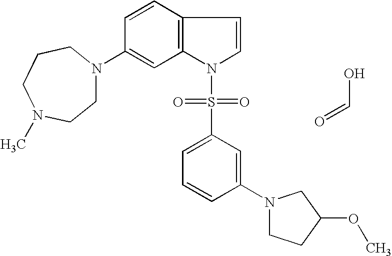 |
| 40 | 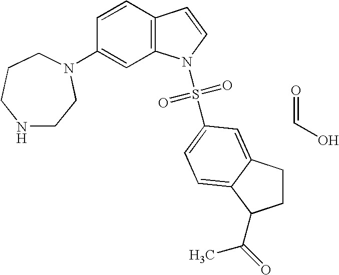 |
| 41 | 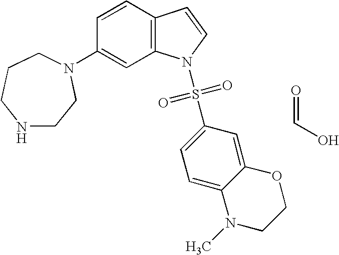 |
| 42 | 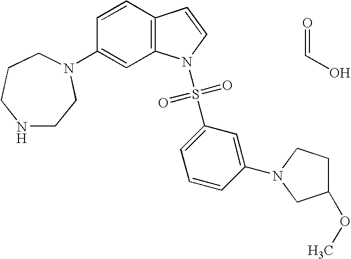 |
| 43 | 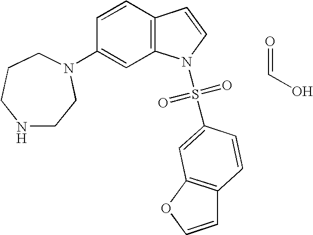 |
| 44 | 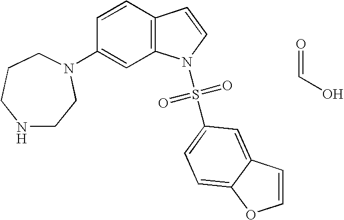 |
| 45 | 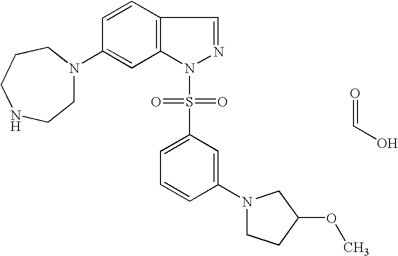 |
| 46 | 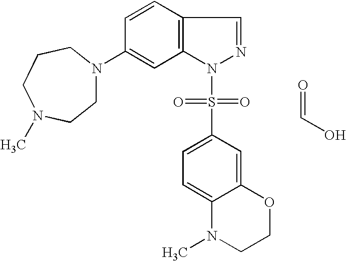 |
| 47 | 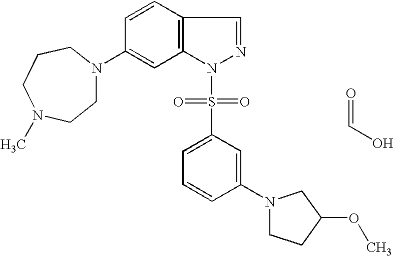 |
| 48 | 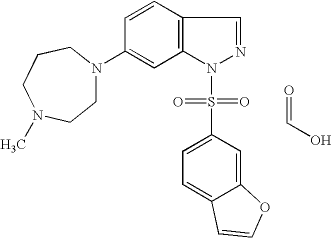 |
| 49 | 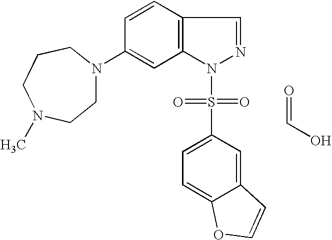 |
| 50 | 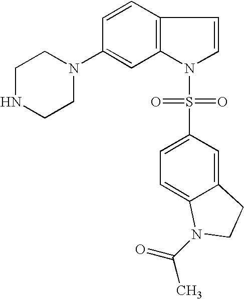 |
| 51 | 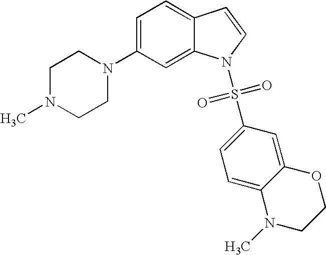 |
| 52 | 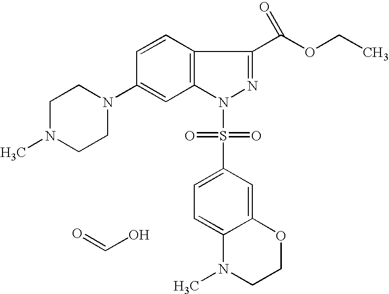 |
| 53 | 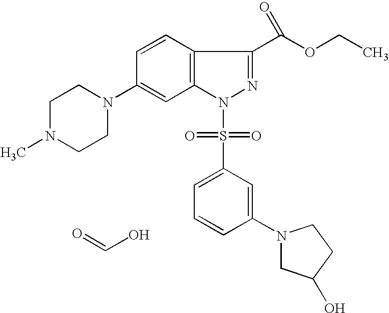 |
| 54 | 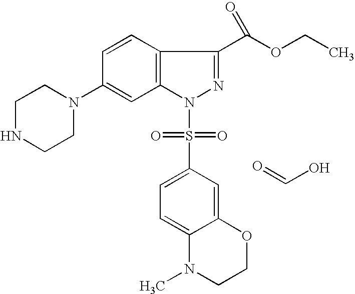 |
| 55 | 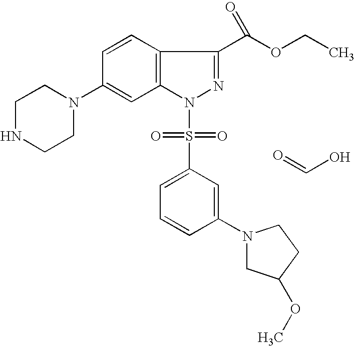 |
| 56 | 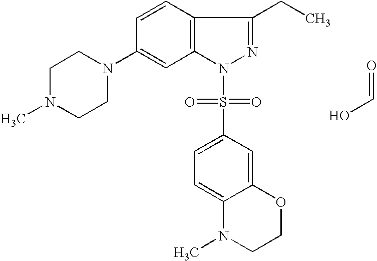 |
| 57 | 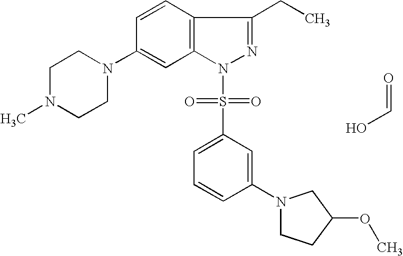 |
| 58 | 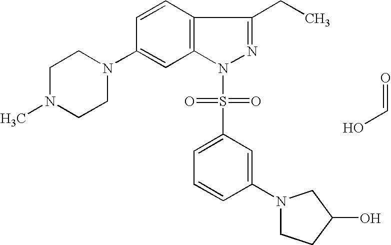 |
| 59 | 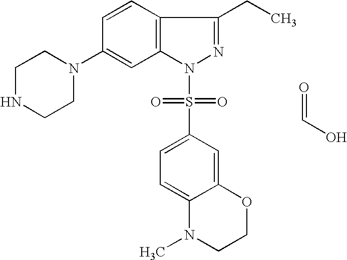 |
| 60 | 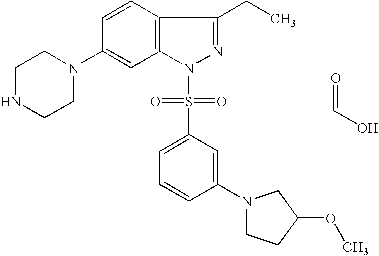 |
| 61 | 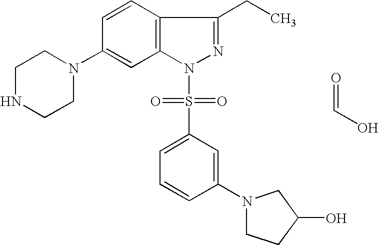 |
| 62 | 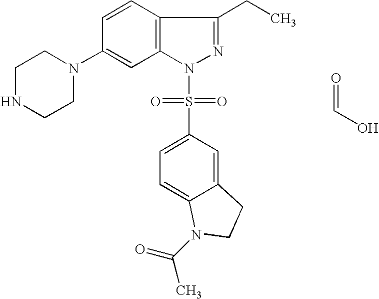 |
| 63 | 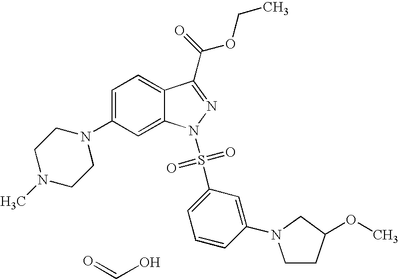 |
| 64 | 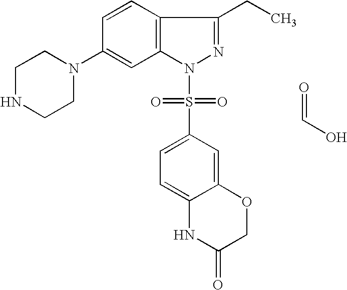 |
| 65 | 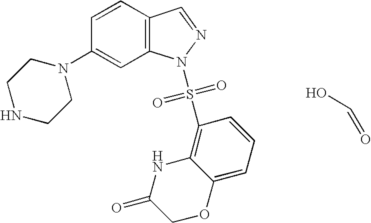 |
| 66 | 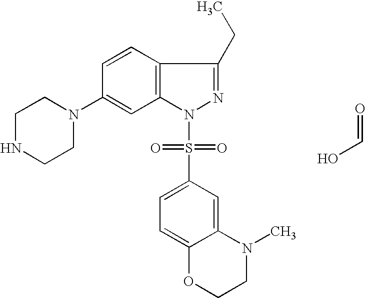 |
| 67 | 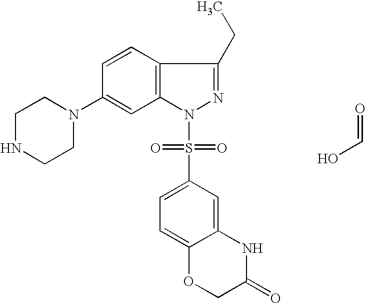 |
| 68 | 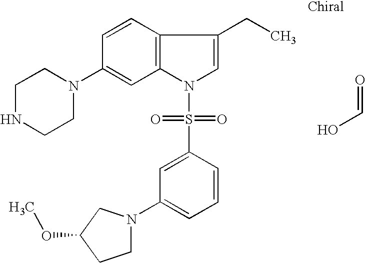 |
| 69 | 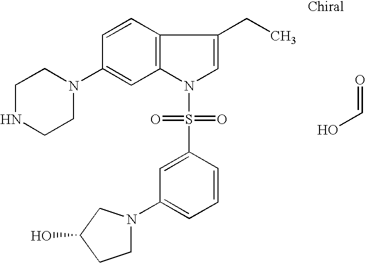 |
| 70 | 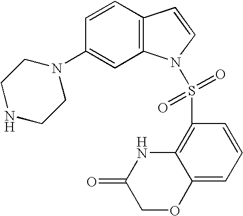 |
| 71 | 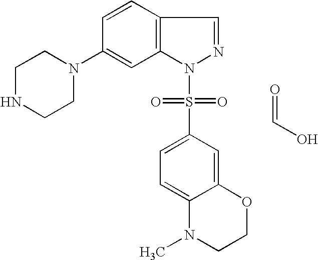 |
| 72 | 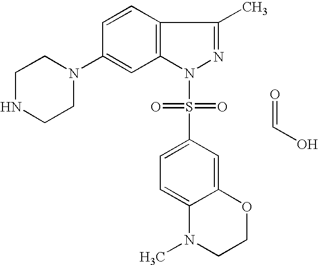 |
| 73 | 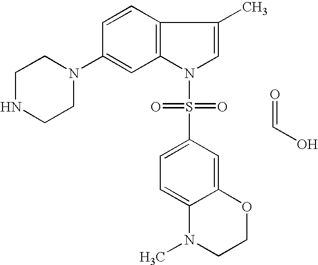 |
| 74 | 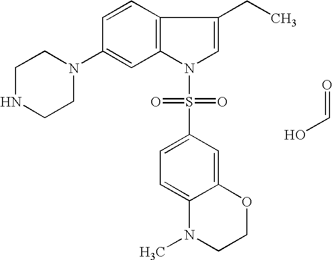 |
| 75 | 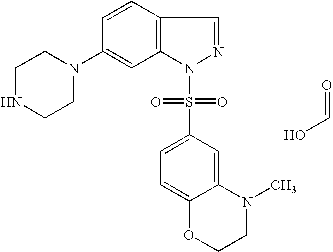 |
| 76 | 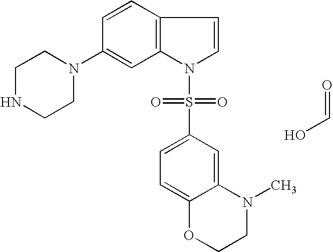 |
| 77 | 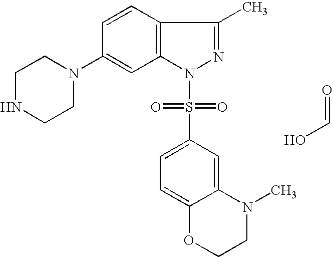 |
| 78 | 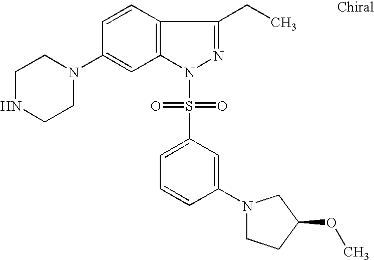 |
| 79 | 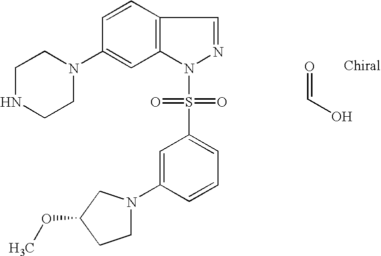 |
| 80 | 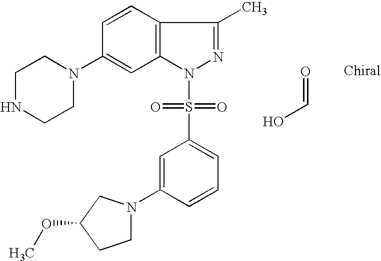 |
| 81 | 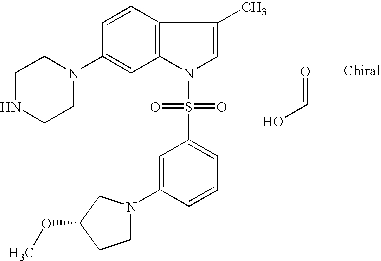 |
| 82 | 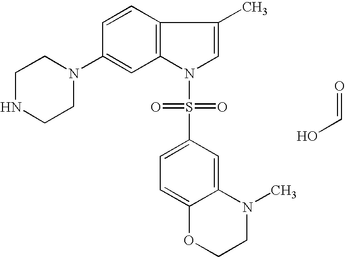 |
| 83 | 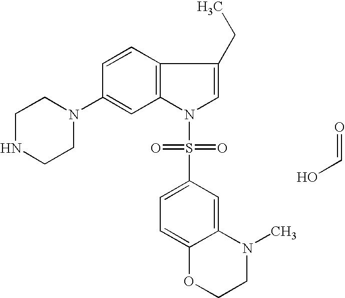 |
| 84 | 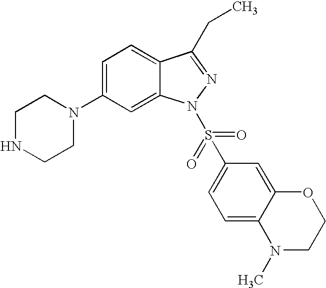 |
| 85 | 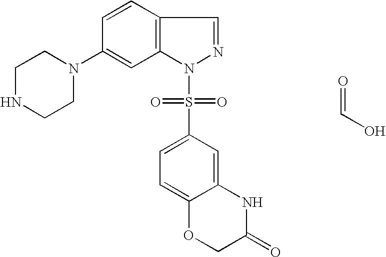 |
| 86 | 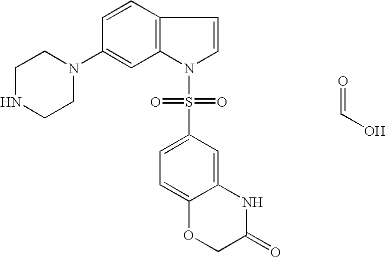 |
| 87 | 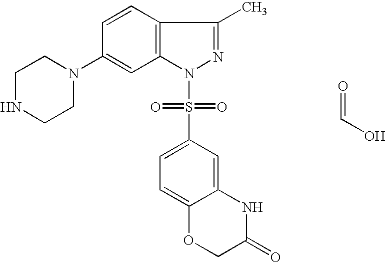 |
| 88 | 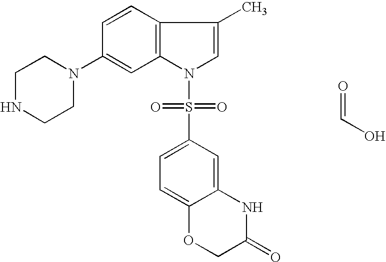 |
| 89 | 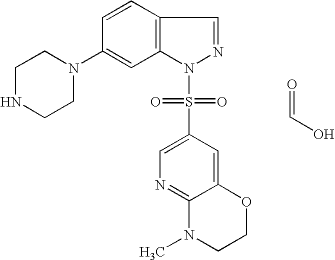 |
| 90 | 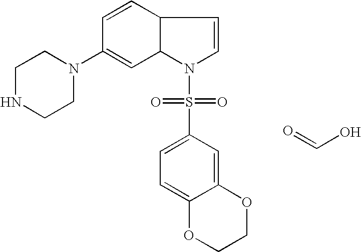 |
| 91 | 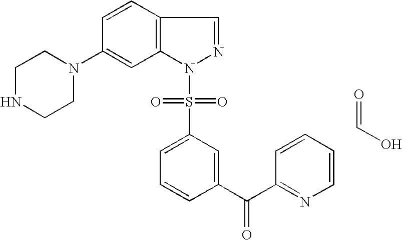 |
| 92 | 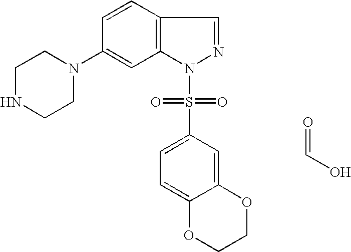 |
| 93 | 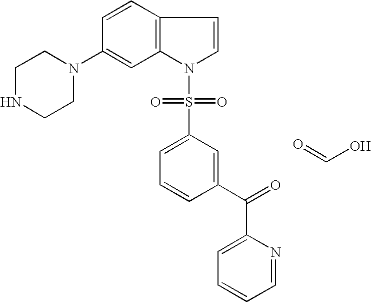 |
| 94 | 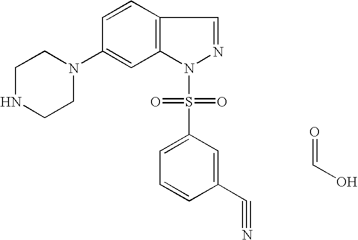 |
| 95 | 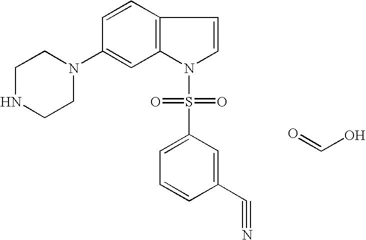 |
| 96 | 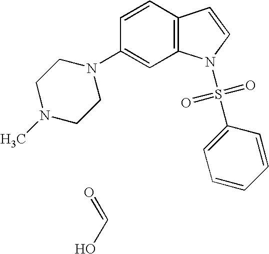 |
| 97 | 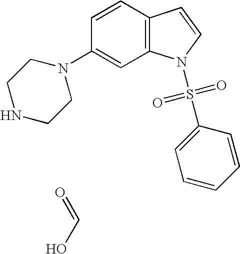 |
| 98 | 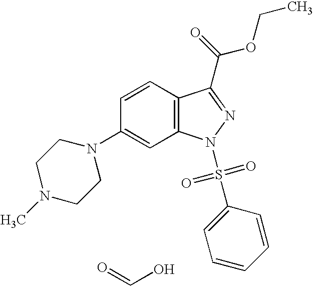 |
| 99 | 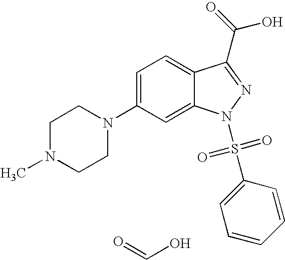 |
| 100 | 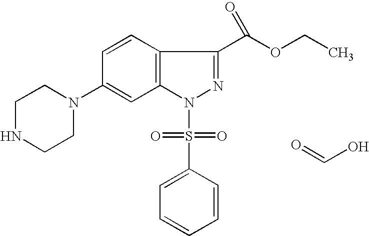 |
| 101 | 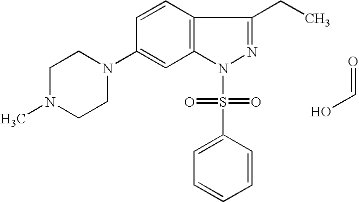 |
| 102 | 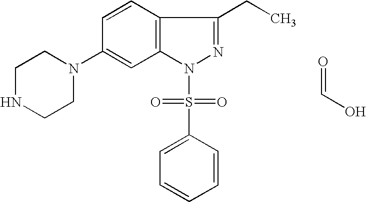 |
| 103 | 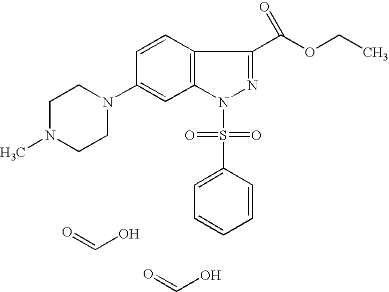 |
| 104 | 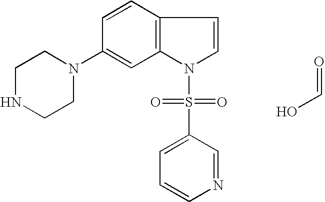 |
| 105 | 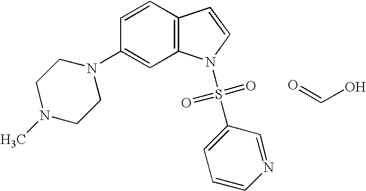 |
| 106 | 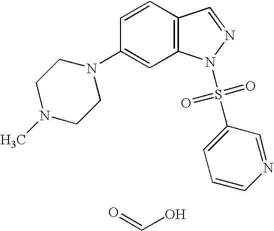 |
| 107 | 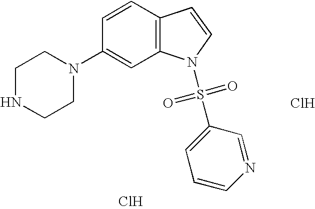 |
| 108 | 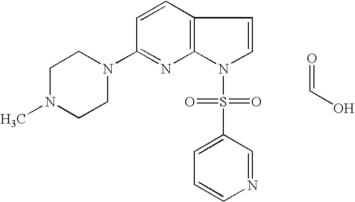 |
| 109 | 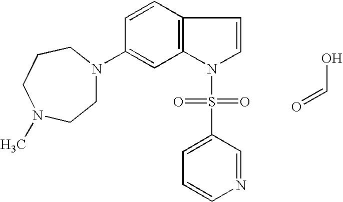 |
| 110 | 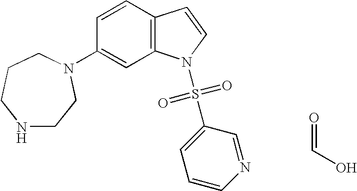 |
| 111 | 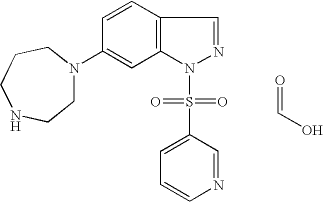 |
| 112 | 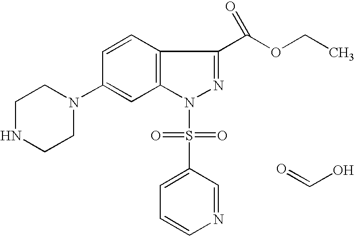 |
| 113 | 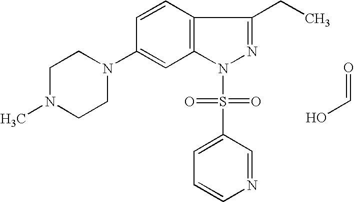 |
| 114 | 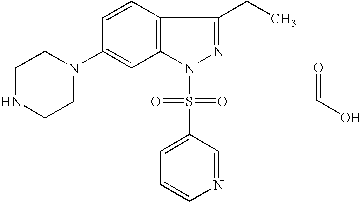 |
| 115 | 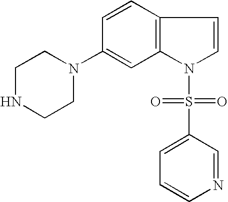 |
| 116 | 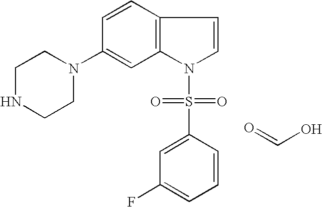 |
| 117 | 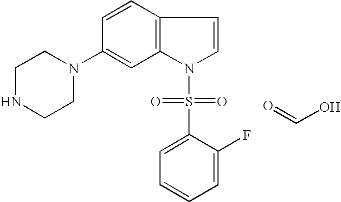 |
| 118 | 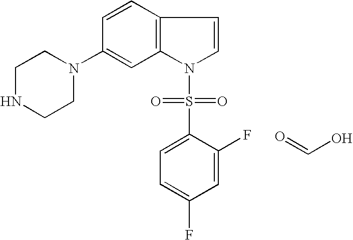 |
| 119 | 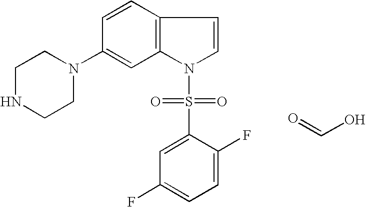 |
| 120 | 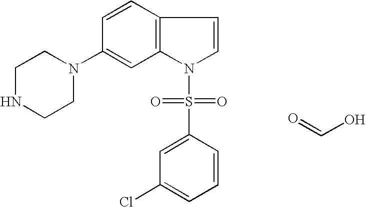 |
| 121 | 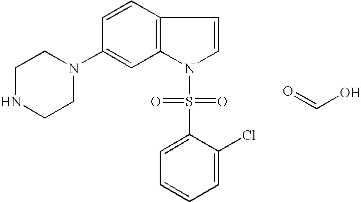 |
| 122 | 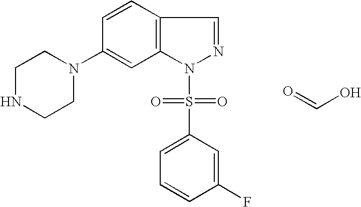 |
| 123 | 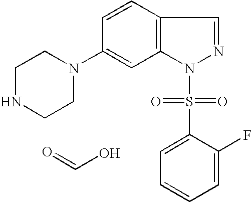 |
| 124 | 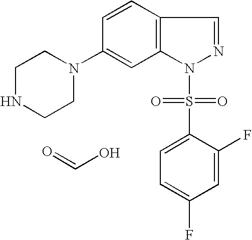 |
| 125 | 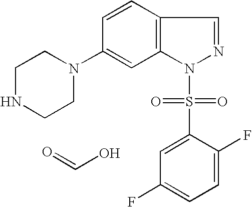 |
| 126 | 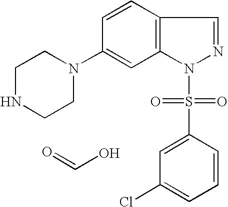 |
| 127 | 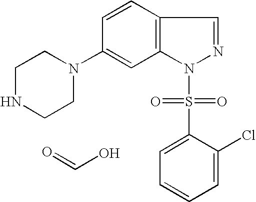 |
| 138 | 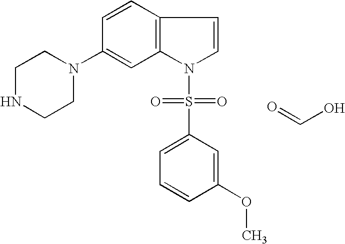 |
| 129 | 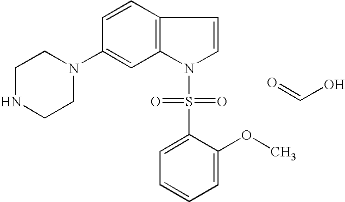 |
| 130 | 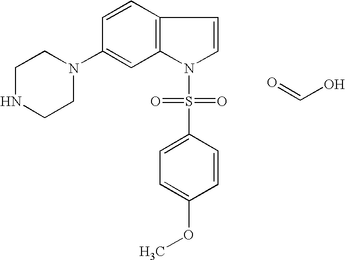 |
| 131 | 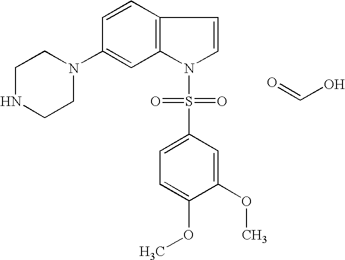 |
| 132 | 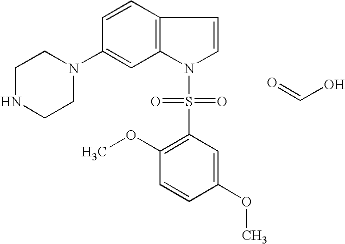 |
| 133 | 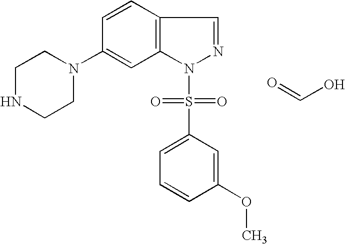 |
| 134 | 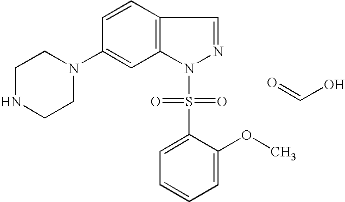 |
| 135 | 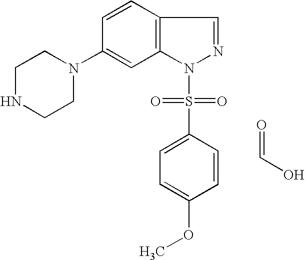 |
| 136 | 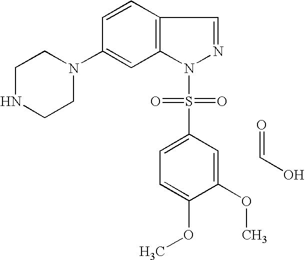 |
| 137 | 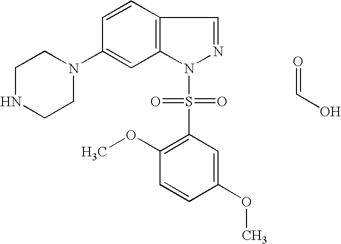 |
| 138 | 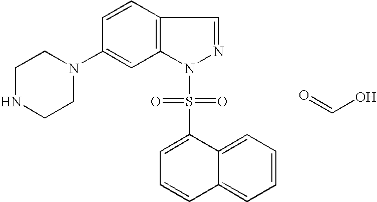 |
| 139 | 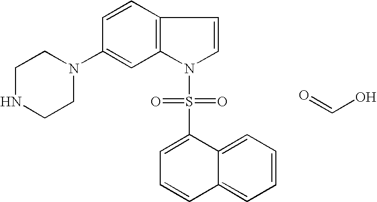 |
| 140 | 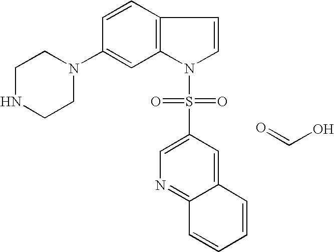 |
| 141 | 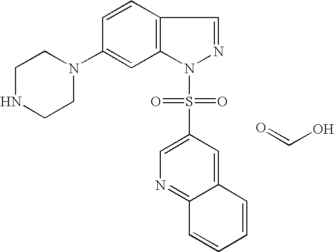 |

Additional aspects of the present invention include pharmaceutical compositions comprising a compound of this invention and a pharmaceutically acceptable carrier and, optionally, one or more additional active agent(s) as discussed below. Further aspects include methods of treating a disease state related to or modulated by the 5-HT6 receptor, in a patient, such as a mammal, e.g., a human, e.g., those disease states mentioned herein.

The compounds of the present invention are effective in inhibiting, or modulating the activity of the 5-HT6 receptor in animals, e.g., mammals, especially humans. These compounds exhibit activity, especially where such activity affects states associated with CNS disorders including motor, mood, personality, behavioral, psychiatric, cognitive, and neurodegenerative disorders, such as, but not limited to, Alzheimer's disease (enhancement of cognitive memory), Parkinson's disease, Huntington's disease, anxiety, depression, manic depression, epilepsy, obsessive compulsive disorders, migraine, sleep disorders, feeding disorders such as anorexia and bulimia, panic attacks, attention deficit hyperactivity disorder (AD1HD), attention deficit disorder (ADD), withdrawal from drug abuse such as cocaine, ethanol, nicotine and benzodiazepines, psychoses, such as schizophrenia, bipolar disorder, and also disorders associated with spinal trauma and/or head injury such as hydrocephalus. Such compounds are also useful for the treatment of memory/cognitive impairment associated with Alzheimer's disease, schizophrenia, Parkinson's disease, Huntington's disease Pick's disease, Creutzfeld Jakob disease, HIV, cardiovascular disease, head trauma or age-related cognitive decline. In addition, such compounds are also expected to be of use in the treatment of certain gastrointestinal (GI) disorders such as, but not limited to, functional bowel disorder, constipation, including chronic constipation, gastroesophageal reflux disease (GERD), noctumal-GERD, and irritable bowel syndrome (IBS), including diarrhea-predominant IBS (IBS-c), constipation-predominant IBS (IBS-c) and alternating constipation/diarrhea IBS.

All methods comprise administering to the patient in need of such treatment an effective amount of one or more compounds of the invention.

A subject or patient in whom administration of the therapeutic compound is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

The compounds of the present invention may be prepared using conventional synthetic methods analogous to those established in the art, and, if required, standard separation or isolation techniques. Suitable synthetic procedures that may be used to prepare the compounds of the present invention are described in, for example, U.S. Pat. Nos. 6,133,217, 6,191,141, and 6,903,112. All starting materials are either commercially available, or can be conventionally prepared from known starting materials without undue experimentation.

One of ordinary skill in the art will recognize that some of the compounds of Formula I can exist in different geometrical isomeric forms. In addition, some of the compounds of the present invention possess one or more asymmetric atoms and are thus capable of existing in the form of optical isomers, as well as in the form of racemic or nonracemic mixtures thereof and in the form of diastereomers and diastereomeric mixtures inter alia. All of these compounds, including cis isomers, trans isomers, diastereomeric mixtures, racemates, nonracemic mixtures of enantiomers, substantially pure, and pure enantiomers, are within the scope of the present invention. Substantially pure enantiomers contain no more than 5% w/w of the corresponding opposite enantiomer, preferably no more than 2%, most preferably no more than 1%.

The optical isomers can be obtained by resolution of the racemic mixtures according to conventional processes, for example, by the formation of diastereomeric salts using an optically active acid or base or formation of covalent diastereomers.

Examples of appropriate acids include, but are not limited to, tartaric, diacetyltartaric, dibenzoyltartaric, ditoluoyltartaric and camphorsulfonic acid. Mixtures of diastereomers can be separated into their individual diastereomers on the basis of their physical and/or chemical differences by methods known to those skilled in the art, for example, by chromatography or fractional crystallization. The optically active bases or acids are then liberated from the separated diastereomeric salts.

A different process for separation of optical isomers involves the use of chiral chromatography (e.g., chiral IULC or SFC columns), with or without conventional derivation, optimally chosen to maximize the separation of the enantiomers. Suitable chiral HPLC columns are manufactured by Diacelt e.g., Chiracel OD and Chiracel OJ among many others, all routinely selectable. Enzymatic separations, with or without derivatization, are also useful. The optically active compounds of Formulas I-H can likewise be obtained by utilizing optically active starting materials in chiral syntheses processes under reaction conditions which do not cause racemization.

In addition, one of ordinary skill in the art will recognize that the compounds can be used in different enriched isotopic forms, e.g., enriched in the content of ²H, ³H, ¹¹C, ¹³C and/or ¹⁴C. In one particular embodiment, the compounds are deuterated. Such deuterated forms can be made by the procedure described in U.S. Pat. Nos. 5,846,514 and 6,334,997. As described in U.S. Pat. Nos. 5,846,514 and 6,334,997, deuteration can improve the efficacy and increase the duration of action of drugs.

Deuterium substituted compounds can be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] (2000), 110 pp. CAN 133:68895 AN 2000:473538 CAPLUS; Kabalka, George W.; Varma, Rajender S. The Synthesis of Radiolabeled Compounds via Organometallic Intermediates. Tetrahedron (1989), 45(21), 6601-21, CODEN: TETRAB ISSN:0040-4020. CAN 112:20527 AN 1990:20527 CAPLUS; and Evans, E. Anthony. Synthesis of radiolabeled compounds, J. Radioanal. Chem. (1981), 64(1-2), 9-32. CODEN: JRACBN ISSN:0022-4081, CAN 95:76229 AN 1981:476229 CAPLUS.

The present invention also relates to useful forms of the compounds as disclosed herein, including free base forms, as well as pharmaceutically acceptable salts or prodrugs of all the compounds of the present invention for which salts or prodrugs can be prepared. Pharmaceutically acceptable salts include those obtained by reacting the main compound, functioning as a base, with an inorganic or organic acid to form a salt for example, but not limited to, salts of hydrochloric acid, sulfuric acid, phosphoric acid, methanesulfonic acid, camphorsulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts are prepared by reacting the compounds of the invention with the appropriate base via a variety of known methods.

The following are further non-limiting examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

For example, the pharmaceutically acceptable salt can be a hydrochloride, hydroformate, hydrobromide, or maleate.

Preferably, the salts formed are pharmaceutically acceptable for administration to mammals. However, pharmaceutically unacceptable salts of the compounds are suitable as intermediates, for example, for isolating the compound as a salt and then converting the salt back to the free base compound by treatment with an alkaline reagent. The free base can then, if desired, be converted to a pharmaceutically acceptable acid addition salt.

One of ordinary skill in the art will also recognize that some of the compounds of Formula I can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

One of ordinary skill in the art will further recognize that compounds of Formula I can exist in different solvate forms. Solvates of the compounds of the invention may also form when solvent molecules are incorporated into the crystalline lattice structure of the compound molecule during the crystallization process. For example, suitable solvates include hydrates, e.g., monohydrates, dihydrates, sesquihydrates, and hemihydrates.

The compounds of the invention can be administered alone or as an active ingredient of a formulation. Thus, the present invention also includes pharmaceutical compositions of one or more compounds of Formula I containing, for example, one or more pharmaceutically acceptable carriers. The compounds of the invention can be administered in a form where the active ingredient is substantially pure.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

In view of their high degree of selective 5-HT6 receptor activity, the compounds of the present invention can be administered to anyone requiring modulation of the 5-HT6 receptor. Administration may be accomplished according to patient needs, for example, orally, nasally, parenterally (subcutaneously, intravenously, intramuscularly, intrasternally and by infusion) by inhalation, rectally, vaginally, topically and by ocular administration.

Various solid oral dosage forms can be used for administering compounds of the invention including such solid forms as tablets, gelcaps, capsules, caplets, granules, lozenges and bulk powders. The compounds of the present invention can be administered alone or combined with various pharmaceutically acceptable carriers, diluents (such as sucrose, mannitol, lactose, starches) and excipients known in the art, including but not limited to suspending agents, solubilizers, buffering agents, binders, disintegrants, preservatives, colorants, flavorants, lubricants and the like. Time release capsules, tablets and gels are also advantageous in administering the compounds of the present invention.

Various liquid oral dosage forms can also be used for administering compounds of the inventions, including aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention. The compounds of the present invention may be injected, for example, intravenously, in the form of an isotonic sterile solution. Other preparations are also possible.

Suppositories for rectal administration of the compounds of the present invention can be prepared by mixing the compound with a suitable excipient such as cocoa butter, salicylates and polyethylene glycols. Formulations for vaginal administration can be in the form of a pessary, tampon, cream, gel, paste, foam, or spray formula containing, in addition to the active ingredient, such suitable carriers as are known in the art.

For topical administration, the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Aerosol formulations suitable for administering via inhalation also can be made. For example, for treatment of disorders of the respiratory tract, the compounds according to the invention can be administered by inhalation in the form of a powder (e.g., micronized) or in the form of atomized solutions or suspensions. The aerosol formulation can be placed into a pressurized acceptable propellant.

The compounds of the present invention are effective in inhibiting, or modulating the activity of the 5-HT6 receptor in animals, e.g., mammals, especially humans. These compounds exhibit activity, especially where such activity affects states associated with CNS disorders including motor, mood, personality, behavioral, psychiatric, cognitive, and neurodegenerative disorders, such as, but not limited to, Alzheimer's disease (enhancement of cognitive memory), Parkinson's disease, Huntington's disease, anxiety, depression, manic depression, epilepsy, obsessive compulsive disorders, migraine, sleep disorders, feeding disorders such as anorexia and bulimia, panic attacks, attention deficit hyperactivity disorder (ADHD), attention deficit disorder (ADD), withdrawal from drug abuse such as cocaine, ethanol, nicotine and benzodiazepines, psychoses, such as schizophrenia, bipolar disorder, and also disorders associated with spinal trauma and/or head injury such as hydrocephalus. Such compounds are also useful for the treatment of memory/cognitive impairment associated with Alzheimer's disease, schizophrenia, Parkinson's disease, Huntington's disease, Pick's disease, Creutzfeld-Jakob disease, WV, cardiovascular disease, head trauma or age-related cognitive decline. In addition, such compounds are also expected to be of use in the treatment of certain gastrointestinal (GI) disorders such as functional bowel disorder and irritable bowel syndrome.

Assays for determining 5-HT6 receptor activity, and selectivity of 5-HT6 receptor activity are known within the art. See, for example, U.S. Pat. Nos. 6,133,287, 6,686,374, and 6,903,112, and Example 13 described below. Compounds of the invention show 5-HT6 binding activity with receptor Ki values of typically less than 1-100 nM. Preferably, the binding activity will be less than 1-50 nM, and more preferably, the activity will be less than 1-10 nM. Compounds of the invention show 5-HT6 functional activity with pA2 values of greater than 6 (IC₅₀ less than 1 μM). Preferably, the pA2 value will be greater than 7 (IC₅₀ less than 500 nM), and more preferably the pA2 value will be greater than 8 (IC₅₀ less than 100 nM).

The preferred pharmacokinetic profile of the compounds may be further shown with measurements to determine HERG and Cyp3A4 inhibition. The HERG inhibition may be measured as described by Dubin, A. (2004). HERG Potassium Channel Activity Assayed with the PatchXpress Planar Patch Clamp. Inaugural PatchXpress User's Meeting, Feb. 12, 2004 (Baltimore, Md.). The Cyp inhibition may be measured as described by Miller V P, Stresser D M, Blanchard A P, Turner S, Crespi C L: Fluorometric high-throughput screening for inhibitors of cytochrome P450. Ann N Y Acad Sci 200; 919:26-32. In one preferred embodiment, the compounds show HERG inhibition with an IC₅₀ greater than 1 μM, preferably greater than 3 μM, and more preferably greater than 10 μM. In another preferred embodiment, the compounds show Cyp3A4 inhibition with an IC₅₀ greater than 1 μM, preferably greater than 3 μM, and more preferably greater than 10 μM.

High HERG inhibition and Cyp3A4 inhibition is potentially linked with adverse cardiac action potential and drug metabolism, respectively.

According to a method aspect, the invention includes a method for the treatment of a disorder of the central nervous system (CNS) related to or affected by the 5-HT6 receptor in a patient in need thereof by administering to the patient a therapeutically effective amount of a compound selected from formula In as described herein above.

The compounds can be administered as the sole active agent or in combination with other pharmaceutical agents such as other agents used in the treatment of CNS disorders, such as psychoses, especially schizophrenia and bipolar disorder, obsessive-compulsive disorder, Parkinson's disease, cognitive impairment and/or memory loss, e.g., nicotinic a-7 agonists, PDE4 inhibitors, PDE10 inhibitors, other 5HT6 receptor ligands, calcium channel blockers, muscarinic m1 and m2 modulators, adenosine receptor modulators, ampakines, NMDA-R modulators, mGluR modulators, dopamine modulators, serotonin modulators, canabinoid modulators, and cholinesterase inhibitors (e.g., donepezil, rivastigimine, and galanthanamine). In such combinations, each active ingredient can be administered either in accordance with their usual dosage range or in accordance with a dose below their usual dosage range.

The compounds can be administered in combination with other pharmaceutical agents used in the treatment of schizophrenia, e.g., Clozaril, Zyprexa, Risperidone, and Seroquel. Thus, the invention also includes methods for treating schizophrenia, including memory impairment associated with schizophrenia, comprising administering to a patient, simultaneously or sequentially, the compound of the invention and one or more additional agents used in the treatment of schizophrenia such as, but not limited to, Clozaril, Zyprexa, Risperidone, and Seroquel. In methods using simultaneous administration, the agents can be present in a combined composition or can be administered separately. As a result the invention also includes compositions comprising a compound according to Formula I and one or more additional pharmaceutical agents used in the treatment of schizophrenia, e.g., Clozaril, Zyprexa, Risperidone, and Seroquel. Similarly, the invention also includes kits containing a composition comprising a compound according to Formula I and another composition comprising one or more additional pharmaceutical agents used in the treatment of schizophrenia, e.g., Clozaril, Zyprexa, Risperidone, and Seroquel.

In addition, the compounds can be administered in combination with other pharmaceutical agents used in the treatment bipolar disorder such as Lithium, Zyprexa, Depakote, and Zyprexa. Thus, the invention also includes methods for treating bipolar disorder, including treating memory and/or cognitive impairment associated with the disease, comprising administering to a patient, simultaneously or sequentially, the compound of the invention and one or more additional agents used in the treatment of bipolar disorder such as, but not limited to, Lithium, Zyprexa, and Depakote. In methods using simultaneous administration, the agents can be present in a combined composition or can be administered separately. As a result, the invention also includes compositions comprising a compound according to Formula I and one or more additional pharmaceutical agents used in the treatment of bipolar disorder such as, but not limited to, Lithium, Zyprexa, and Depakote. Similarly, the invention also includes kits containing a composition comprising a compound according to Formula I and another composition comprising one or more additional pharmaceutical agents used in the treatment of bipolar disorder such as Lithium, Zyprexa, and Depakote.

In one preferred embodiment, the compounds of the invention can be administered in combination with a nicotinic acetylcholine subtype α-7 receptor ligand (a-7 receptor ligand). Nicotinic acetylcholine subtype α-7 receptor ligands modulate the function of nicotinic acetylcholine subtype α-7 receptors by altering the activity of the receptor. Suitable compounds also can be partial agonists that partially block or partially activate the α-7 receptor or agonists that activate the receptor. Positive allosteric modulators are compounds that potentiate the receptor response to acetylcholine without themselves triggering receptor activation or desensitization, or either, of the receptor. Nicotinic acetylcholine subtype α7 receptor ligands that can be combined with the 5HT6 ligand of the present invention can include full agonists, partial agonists, or positive allosteric modulators.

α-7 receptor ligands typically demonstrate Kᵢ values from about 1 nM to about 10 μM when tested by the [³H1-MLA assay. Many having a binding value (“Kᵢ MLA”) of less than 1 μM. According to one embodiment, [3H]-Cytisine binding values (“Kᵢ Cyt”) of the α-7 receptor ligand range from about 50 nM to greater than 100 μM. According to another embodiment, preferred α-7 receptor ligands have Kᵢ MLA value (as measured by MLA assay in view of the Kᵢ Cyt value as measured by (3H]-cytisine binding, such that in the formula D=Kᵢ Cyt/Kᵢ MLA) of at least 50. For example, preferred compounds typically exhibit greater potency at α-7 receptors compared to α4β2 receptors. Although the MLA and [3H]-cytisine binding assays are well known, further details for carrying out the assays are provided in International Publication Nos. WO 2005/028477; WO 2005/066168; US 20050137184; US20050137204; US2005024553 1; WO 2005/066166; WO 2005/066167; and WO 2005/077899.

Positive allosteric modulators, at concentrations ranging from 1 nM to 10 μM, enhance responses of acetylcholine at α-7 nicotinic receptors expressed endogenously in neurons or cell lines, or via expression of recombinant protein in Xenopus oocytes or in cell lines. α-7 receptor ligands can be used to improve efficacy of 5HT6 ligands without exaggerating the side effect profile of such agents.

Accordingly, α-7 receptor ligands that may be combined with the 5HT6 ligand can be compounds of various chemical classes. Particularly, some examples of α-7 receptor ligands suitable for the invention include, but are not limited to, diazabicycloalkane derivatives, for example as described in International Publication No. WO 2005/028477; spirocyclic quinuclidinic ether derivatives, for example as described in International Publication No. WO 2005/066168; fused bicycloheterocycle substituted quinuclidine derivatives, for example as described in US Publication Nos. US20050137184; US20050137204; and US20050245531; 3-quinuclidinyl aminosubstituted biaryl derivatives, for example as described in International Publication No. WO 2005/066166; 3-quinuclidinyl heteroatom-bridged biaryl derivatives, for example as described in International Publication No. WO 2005/066167; and aminosubstituted tricyclic derivatives, for example as described in International Publication No. WO 2005/077899, all of which are hereby incorporated by reference in their entirety.

Examples of compounds reported as α-7 agonists or partial agonists are quinuclidine derivatives, for example as described in WO 2004/016608 and WO 2004/022556; and tilorone derivatives, for example also as described in WO 2004/016608.

Examples of compounds reported as positive allosteric modulators are 5-hydroxyindole analogs, for example as described in WO 01/32619, WO 01/32620, and WO 01/32622; tetrahydroquinoline derivatives, for examples as described in WO 04/098600; amino-thiazole derivatives; and diarylurea derivatives, for example as described in WO 04/085433.

Specific examples of compounds that are suitable neuronal nicotinic subtype α-7 receptor ligands include, for example, 5-(6-[(3R)-1-azabicyclo[2.2.2]oct-3-yloxy]pyridazin-3-yl)-1H-indole; 2-(6-phenylpyridazine-3-yl)octahydropyrrolol-[3,4-c]pyrrole; 5-[5-{(1R,5R)-6-methyl-3,6-diaza-bicyclo[[3.2.0]hept-3-yl}-pyridin-2-yl]-1H-indole; and 5-[6-(cis-5-methyl-hexahydro-pyrrolo[3,4-c]pyrrol-2-yl)-pyridazin-3-yl-1H-indole. Other suitable α-7 ligands are described in WO2006/101745, which is hereby incorporated by reference.

Compounds modulating activity of nicotinic acetylcholine receptor α-7 subtype are suitable for the invention regardless of the manner in which they affect the receptor. Other compounds reported as demonstrating α-7 activity include, but are not limited to, quinuclidine amide derivatives, for example PNU-282987, N-[(3R)-1-azabicyclo[2.2.2]oct-3-yl]-4-chlorobenzamide TC-5619, varanicline, and others as described in WO 04/052894, and MEM-3454. Additional compounds can include, but are not limited to, AR R17779, AZD0328, WB-56203, SSR-18071 IA, GTS21, and OH-GTS-21, which are all described in the publicly available literature.

The invention also includes methods for treating Parkinson's disease, including treating memory and/or cognitive impairment associated with Parkinson's disease, comprising administering to a patient, simultaneously or sequentially, the compound of the invention and one or more additional agents used in the treatment of Parkinson's disease such as, but not limited to, Levodopa, Parlodel, Permax, Mirapex, Tasmar, Contan, Kemadin, Artane, and Cogentin. In methods using simultaneous administration, the agents can be present in a combined composition or can be administered separately. As a result, the invention also includes compositions comprising a compound according to Formula I and one or more additional pharmaceutical agents used in the treatment of Parkinson's disease, such as, but not limited to, Levodopa, Parlodel, Permax, Mirapex, Tasmar, Contan, Kemadin, Artane, and Cogentin. Similarly, the invention also includes kits containing a composition comprising a compound according to Formula I and another composition comprising one or more additional pharmaceutical agents gent used in the treatment of Parkinson's disease such as, but not limited to, Levodopa, Parlodel, Permax, Mirapex, Tasmar, Contan, Kemadin, Artane, and Cogentin.

In addition, the invention includes methods for treating memory and/or cognitive impairment associated with Alzheimer's disease comprising administering to a patient, simultaneously or sequentially, the compound of the invention and one or more additional agents used in the treatment of Alzheimer's disease such as, but not limited to, Reminyl, Cognex, Aricept, Exelon, Akatinol, Neotropin, Eldepryl, Estrogen and Cliquinol. In methods using simultaneous administration, the agents can be present in a combined composition or can be administered separately. As a result, the invention also includes compositions comprising a compound according to Formula I and one or more additional pharmaceutical agents used in-the treatment of Alzheimer's disease such as, but not limited to, Reminyl, Cognex, Aricept, Exelon, Akatinol, Neotropin, Eldepryl, Estrogen and Cliquinol. Similarly, the invention also includes kits containing a composition comprising a compound according to Formula I and another composition comprising one or more additional pharmaceutical agents used in the treatment of Alzheimer's disease such as, but not limited to Reminyl, Cognex, Aricept, Exelon, Akatinol, Neotropin, Eldepryl, Estrogen and Cliquinol.

Another aspect of the invention includes methods for treating memory and/or cognitive impairment associated with dementia comprising administering to a patient, simultaneously or sequentially, the compound of the invention and one or more additional agents used in the treatment of dementia such as, but not limited to, Thioridazine, Haloperidol, Risperidone, Cognex, Aricept, and Exelon. In methods using simultaneous administration, the agents can be present in a combined composition or can be administered separately. As a result, the invention also includes compositions comprising a compound according to Formula I and one or more additional pharmaceutical agents used in the treatment of dementia such as, but not limited to, Thioridazine, Haloperidol, Risperidone, Cognex, Aricept, and Exelon. Similarly, the invention also includes kits containing a composition comprising a compound according to Formula I and another composition comprising one or more additional pharmaceutical agents used in the treatment of dementia such as, but not limited to, Thioridazine, Haloperidol, Risperidone, Cognex, Aricept, and Exelon.

A further aspect of the invention includes methods for treating memory and/or cognitive impairment associated with epilepsy comprising administering to a patient, simultaneously or sequentially, the compound of the invention and one or more additional agents used in the treatment of epilepsy such as, but not limited to, Dilantin, Luminol, Tegretol, Depakote, Depakene, Zarontin, Neurontin, Barbita, Solfeton, and Felbatol. In methods using simultaneous administration, the agents can be present in a combined composition or can be administered separately. As a result, the invention also includes compositions comprising a compound according to Formula I and one or more additional pharmaceutical agents used in the treatment of epilepsy such as, but not limited to, Dilantin, Luminol, Tegretol, Depakote, Depakene, Zarontin, Neurontin, Barbita, Solfeton, and Felbatol. Similarly, the invention also includes kits containing a composition comprising a compound according to Formula I and another composition comprising one or more additional pharmaceutical agents used in the treatment of epilepsy such as, but not limited to, Dilantin, Luminol, Tegretol, Depakote, Depakene, Zarontin, Neurontin, Barbita, Solfeton, and Felbatol.

A further aspect of the invention includes methods for treating memory and/or cognitive impairment associated with multiple sclerosis comprising administering to a patient, simultaneously or sequentially, the compound of the invention and one or more additional agents used in the treatment of multiple sclerosis such as, but not limited to, Detrot, Ditropan XL, OxyContin, Betaseron, Avonex, Azothioprine, Methotrexate, and Copaxone. In methods using simultaneous administration, the agents can be present in a combined composition or can be administered separately. As a result, the invention also includes compositions comprising a compound according to Formula I and one or more additional pharmaceutical agents used in the treatment of multiple sclerosis such as, but not limited to, Detrol, Ditropan XL, OxyContin, Betaseron, Avonex, Azothioprine, Methotrexate, and Copaxone. Similarly, the invention also includes kits containing a composition comprising a compound according to Formula I and another composition comprising one or more additional pharmaceutical agents used in the treatment of multiple sclerosis such as, but not limited to, Detrol, Ditropan XL, OxyContin, Betaseron, Avonex, Azothioprine, Methotrexate, and Copaxone.

The invention further includes methods for treating Huntington's disease, including treating memory and/or cognitive impairment associated with Huntington's disease, comprising administering to a patient, simultaneously or sequentially, the compound of the invention and one or more additional agents used in the treatment of Huntington's disease such as, but not limited to, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, and Risperidone. In methods using simultaneous administration, the agents can be present in a combined composition or can be administered separately. As a result, the invention also includes compositions comprising a compound according to Formula I and one or more additional pharmaceutical agents used in the treatment of Huntington's disease such as, but not limited to, Amitriptyline, Imipramine, Despiramine, Nortriptyline, Paroxetine, Fluoxetine, Setraline, Terabenazine, Haloperidol, Chloropromazine, Thioridazine, Suipride, Quetiapine, Clozapine, and Risperidone. Similarly, the invention also includes kits containing a composition comprising a compound according to Formula I and another composition comprising one or more additional pharmaceutical agents used in the treatment of Huntington's disease such as, but not limited to, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, and Risperidone.

Indications that may be treated with 5HT6 ligands, either alone or in combination with other drugs, include, but are not limited to, those diseases thought to be mediated in part by the basal ganglia, prefrontal cortex and hippocampus. These indications include psychoses, Parkinson's disease, dementias, obsessive compulsion disorder, tardive dyskinesia, choreas, depression, mood disorders, impulsivity, drug addiction, attention deficit/hyperactivity disorder (ADED), depression with parkinsonian states, personality changes with eaudate or putamen disease, dementia and mania with caudate and pallidal diseases, and compulsions with pallidal disease.

Psychoses are disorders that affect an individual's perception of reality. Psychoses are characterized by delusions and hallucinations. The present invention includes methods for treating patients suffering from all forms of psychoses, including but not limited to schizophrenia, late-onset schizophrenia, schizoaffective disorders, prodromal schizophrenia, and bipolar disorders. Treatment may be for the positive symptoms of schizophrenia as well as for the cognitive deficits and negative symptoms. Other indications for 5-HT6 ligands include psychoses resulting from drug abuse (including amphetamines and PCP), encephalitis, alcoholism, epilepsy, Lupus, sarcoidosis, brain tumors, multiple sclerosis, dementia with Lewy bodies, or hypoglycemia. Other psychiatric disorders, like posttraumatic stress disorder (PTSD), and schizoid personality may also be treated with 5-HT6 ligands.

Dementias are diseases that include memory loss and additional intellectual impairment separate from memory. The present invention includes methods for treating patients suffering from memory impairment in all forms of dementia. Dementias are classified according to their cause and include: neurodegenerative dementias (e.g., Alzheimer's, Parkinson's disease, Huntington's disease, Pick's disease), vascular (e.g., infarcts, hemorrhage, cardiac disorders), mixed vascular and Alzheimer's, bacterial meningitis, Creutzfeld-Jacob Disease, multiple sclerosis, traumatic (e.g., subdural hematoma or traumatic brain injury), infectious (e.g., HIV), genetic (Down syndrome), toxic (e.g., heavy metals, alcohol, some medications), metabolic (e.g., vitamin B12 or folate deficiency), CNS hypoxia, Cushing's disease, psychiatric (e.g., depression and schizophrenia), and hydrocephalus.

The condition of memory impairment is manifested by impairment of the ability to learn new information and/or the inability to recall previously learned information. The present invention includes methods for dealing with memory loss separate from dementia, including mild cognitive impairment (MCI) and age-related cognitive decline. The present invention includes methods of treatment for memory impairment as a result of disease. Memory impairment is a primary symptom of dementia and can also be a symptom associated with such diseases as Alzheimer's disease, schizophrenia, Parkinson's disease, Huntington's disease, Pick's disease, Creutzfeld-Jakob disease, ETV, cardiovascular disease, and head trauma as well as age-related cognitive decline. In another application, the invention includes methods for dealing with memory loss resulting from the use of general anesthetics, chemotherapy, radiation treatment, post-surgical trauma, and therapeutic intervention. Thus, in accordance with a preferred embodiment, the present invention includes methods of treating patients suffering from memory impairment due to, for example, Alzheimer's disease, multiple sclerosis, amylolaterosclerosis (ALS), multiple systems atrophy (MSA), schizophrenia, Parkinson's disease, Huntington's disease, Pick's disease, Creutzfeld-Jakob disease, depression, aging, head trauma, stroke, spinal cord injury, CNS hypoxia, cerebral senility, diabetes associated cognitive impairment, memory deficits from early exposure of anesthetic agents, multiinfarct dementia and other neurological conditions including acute neuronal diseases, as well as WHV and cardiovascular diseases. The invention also relates to agents and/or methods to stimulate the formation of memory in “normal” subjects (i.e., subjects who do not exhibit an abnormal or pathological decrease in a memory function), e.g., ageing middle-aged subjects.

The invention is also suitable for use in the treatment of a class of disorders known as polyglutamine-repeat diseases. These diseases share a common pathogenic mutation. The expansion of a CAG repeat, which encodes the amino acid glutamine, within the genome leads to production of a mutant protein having an expanded polyglutamine region. For example, Huntington's disease has been linked to a mutation of the protein huntingtin. In individuals who do not have Huntington's disease, huntingtin has a polyglutamine region containing about 8 to 31 glutamine residues. For individuals who have Huntington's disease, huntingtin has a polyglutamine region with over 37 glutamine residues. Aside from Huntington's disease (HD), other known polyglutamine-repeat diseases and the associated proteins are: dentatorubral-pallidoluysian atrophy, DRPLA (atrophin-1); spinocerebellar ataxia type-1 (ataxin-1); spinocerebellar ataxia type-2 (ataxin-2); spinocerebellar ataxia type-3 also called Machado-Joseph disease, MJD (ataxin-3); spinocerebellar ataxia type-6 (alpha la-voltage dependent calcium channel); spinocerebellar ataxia type-7 (ataxin-7); and spinal and bulbar muscular atrophy, SBMA, also known as Kennedy disease (androgen receptor). Thus, in accordance with a further aspect of the invention, there is provided a method of treating a polyglutamine-repeat disease or CAG repeat expansion disease comprising administering to a patient, such as a mammal, especially a human, a therapeutically effective amount of a compound. In accordance with a further embodiment, there is provided a method of treating Huntington's disease (HD), dentatorubral-pallidoluysian atrophy (DRPLA), spinocerebellar ataxia type-1, spinocerebellar ataxia type-2, spinocerebellar ataxia type-3 (Machado-Joseph disease), spinocerebellar ataxia type-6, spinocerebellar ataxia type-7, or spinal and bulbar muscular atrophy, comprising administering to a patient, such as a mammal, especially a human, a therapeutically effective amount of a compound of the invention.

The basal ganglia are important for regulating the function of motor neurons; disorders of the basal ganglia result in movement disorders. Most prominent among the movement disorders related to basal ganglia function is Parkinson's disease (Obeso J A et at., Neurology., Jan. 13, 2004;62(1 Suppl 1):517-30). Other movement disorders related to dysfunction of the basla ganglia include tardive dyskinesia, progressive supranuclear palsy and cerebral palsy, corticobasal degeneration, multiple system atrophy, Wilson disease, and dystonia, tics, and chorea. In one embodiment, the compounds of the invention may be used to treat movement disorders related to dysfunction of basal ganglia neurons.

The dosages of the compounds of the present invention depend upon a variety of factors including the particular syndrome to be treated, the severity of the symptoms, the route of administration, the frequency of the dosage interval, the particular compound utilized, the efficacy, toxicology profile, pharmacokinetic profile of the compound, and the presence of any deleterious side-effects, among other considerations. One of ordinary skill in the art of treating such diseases will be able, without undue experimentation and in reliance upon personal knowledge and the disclosure of this Application, to ascertain a therapeutically effective amount of the compounds of the present invention for a given disease.

The compounds of the invention are typically administered at dosage levels and in a mammal customary for 5-HT6 ligands, such as those known compounds mentioned above. For example, the compounds can be administered, in single or multiple doses, by oral administration at a dosage level of generally 0.001-100 mg/kg/day, for example, 0.01-100 mg/kg/day, preferably 0.1-70 mg/kg/day, especially 0.5-10 mg/kg/day. Unit dosage forms can contain generally 0.01-1000 mg of active compound, for example, 0.1-50 mg of active compound. For intravenous administration, the compounds can be administered, in single or multiple dosages, at a dosage level of, for example, 0.001-50 mg/kg/day, preferably 0.001-10 mg/kg/day, especially 0.01-1 mg/kg/day. Unit dosage forms can contain, for example, 0.1-10 mg of active compound.

In carrying out the procedures of the present invention, it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

The present invention will now be further described by way of the following non-limiting examples. In applying the disclosure of these examples, it should be kept clearly in mind that other and different embodiments of the methods disclosed according to the present invention will no doubt suggest themselves to those of skill in the relevant art.

In the foregoing and in the following examples, all temperatures are set forth uncorrected in degrees Celsius; and, unless otherwise indicated, all parts and percentages are by weight.

The entire disclosures of all applications, patents and publications, cited above and below, are hereby incorporated by reference in their entirety.

### EXAMPLES

All spectra were recorded at 300 MHz on a Bruker Instruments NMR unless otherwise stated. Coupling constants (J) are in Hertz (Hz) and peaks are listed relative to TMS (δ 0.00 ppm).

Analytical UPLC was performed on (i) 4.0 mm×50 mm WATERS YMC ODS-A Cartridge 120A S3u 4 column using a gradient of 0/100 to 100/0 acetonitrile (0.05% TFA)/water (0.05% TFA) over 4 min (for all compounds except 1-[(1-acetyl-2,3-dihydro-1H-indol-5-yl)sulfonyl]-3-(1-methyl-1,2,3,6-tetrahydropyridin-4-yl)-1H-indole, or (ii) a 4.6 mm×100 mm Waters Sunfire™ RP C18 5 mm column using a gradient of 20/80 to 80/20 acetonitrile (0.1% formic acid)/water (0.1% formic acid) over 8 min. This procedure is written as (2080_{—}8min).

Additional HPLC analysis is performed on (iii) a 4.6 mm×100 mm Waters Sunfire™ RP C18 5 mm column using a constant flow of 80/20 acetonitrile (0.1% formic acid)/water (0.1% formic acid) over 8 min. This procedure is written as (8080_{—}8 min).

Preparative HPLC was performed on 30 mm×100 mm Xterra Prep RPIR 5μ columns using an 8 min gradient of 95/5 to 20/80 water (0.1% formic acid)/acetonitrile (0.1% formic acid).

Acronyms and abbreviations used in the experimental descriptions are as follows:
Ac acetyl
AcCl acetyl chloride
aq aqueous
BINAP 2,2′-bis(diphenylphosphino-1,1′-binaphthyl (ligand)
Boc tert-butylcarbonyloxy
Bu butyl
n-BuLi n-butyllithium
calcd calculated
conc concentrated
Cbz carbobenzoxy
d doublet
DCM dichloromethane (methylene chloride)
dd doublet of doublet
ddd doublet of doublet of doublet
DEAD diethylazodicarboxylate
DMF N,N-dimethyl formamide
DMSO dimethylsulfoxide
DMSO-d₆ dimethylsulfoxide-d₆
equiv equivalent
ES-MS electrospray mass spectrometry
Et ethyl
Et₂O diethyl ether
Et₃N triethylamine
EtOAc ethyl acetate
EtOH ethanol
g gram
GC-MS gas chromatography-mass spectrometry
h hour(s)
¹H NMR proton nuclear magnetic resonance
f UNO₃ fuming nitric acid
HOAc acetic acid
HPLC high-performance liquid chromatography
KOAc potassium acetate
L liter
LCMS liquid chromatography / mass spectroscopy
m multiplet
M molar
mL milliliter
m/z mass over charge
Me methyl
MeI iodomethane
MeOH methanol
mg milligram
MHz megahertz
min minute(s)
mmol millimole
mol mole
mp melting point
MS mass spectrometry
N normal
NBS N-bromosuccinimide
NCS N-chlorosuccinimide
NMR nuclear magnetic resonance
Pd(OAc)₂ palladium acetate
Pd(PPh₃)₄-( tetrakis(triphenylphosphine)palladium(0)
Pd/C palladium on carbon
PE petroleum ether
Ph phenyl
ppm parts per million
Pr propyl
i-PrOH isopropanol (2-propanol)
Py pyridine
q quartet
qt quintet
rt room temperature
s singlet
sat saturated
t triplet
TEBA N-benzyl-N-chloro-N,N-diethylethanamine; (triethylbenzylammonium chloride)
TFA trifluoroacetic acid
THF tetrahydrofuran
TLC thin layer chromatography
TMS tetramethylsilane
P-TSA p-toluenesulfonic acid
V/V volume per unit volume
vol volume
w/w weight per unit weight

Preparative HPLC was performed on 30 mm×100 mm Xterra Prep RP18 5μ columns using an 8 min gradient of 95/5 to 20/80 water (0.1% formic acid)/acetonitrile (0.1% formic acid).

### Experimental Details

### General Procedure A

### Synthesis of tert-butyl 4-(1H-indol-6-yl)piperazine-1-carboxylate

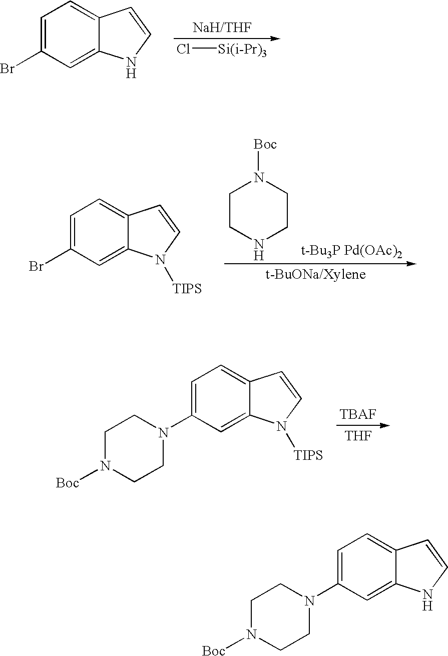

### Synthesis of 6-bromo-1-(triisopropylsilyl)-1H-indole

Into a 500 mL 3-necked round-bottom flask, was placed a solution of NaH (3.36 g, 84.00 mmol) in THF (100 mL) The temperature was cooled to 0° C. This was followed by the addition of a solution of 6-bromo-1H-indole (15 g, 76.51 mmol) in TIE (100 mL), which was added dropwise with stirring, while cooling to a temperature of 0° C. over a time period of 30 minutes. To the mixture was added chlorotriisopropylsilane (17.7 g, 91.80 mmol). The resulting solution was allowed to react, with stirring, for 20 minutes while the temperature was maintained at room temperature. The reaction progress was monitored by TLC (ethyl acetate/petroleum ether=1:10). The mixture was concentrated by evaporation under vacuum using a rotary evaporator. The resulting solution was diluted with 300 mL of ethyl acetate. The resulting mixture was washed 2 times with 100 mL of brine. The mixture was dried over Na₂SO₄. The residue was purified by eluting through a column with a petroleum ether solvent system. This resulted in 25 g (93%) of 6-bromo-1-(triisopropylsilyl)-1H-indole as a yellow liquid.

### 2. Synthesis of tert-butyl 4-(1-(triisopropylsilyl)-1H-indol-6-yl) piperazine-1-carboxylate

Into a 100 mL three necked flask purged and maintained with an inert atmosphere of nitrogen, was placed a solution of 6-bromo-1-(triisopropylsilyl)-1H-indole (5 g, 14.19 mmol) in Xylene (50 mL). To this was added tert-butyl piperazine-1-carboxylate (7.95 g, 42.68 mmol). Addition of t-BuONa (1.9 g, 19.77 mmol) was next. This was followed by the addition of tri-tert-butylphosphine (580 mg, 2.87 mmol). To the mixture was added Pd(OAc)2 (160 mg, 0.71 mmol). The resulting solution was allowed to react, with stirring, for 30 minutes while the temperature was maintained at 120° C. The reaction progress was monitored by TLC (ethyl acetate/petroleum ether=1:5).The mixture was concentrated by evaporation under vacuum using a rotary evaporator. The resulting solution was diluted with 250 mL of ethyl acetate. The resulting mixture was washed 2 times with 150 mL of brine. The mixture was dried over Na₂SO₄. The residue was purified by eluting through a column with a 1:40 ethyl acetate/petroleum ether solvent system. This resulted in 5 g (73%) of tert-butyl 4-(1-(triisopropylsilyl)-1H-indol-6-yl)piperazine-1-carboxylate as a yellow solid.

### 3. Synthesis of tert-butyl 4-(1H-indol-6-yl)piperazine-1-carboxylate

Into a 250 mL round-bottom flask, was placed a solution of tert-butyl 4-(1-(triisopropylsilyl)-1H-indol-6-yl)piperazine-1-carboxylate (6 g, 13.13 mmol) in THF (100 mL). To the mixture was added TBAF (3.43 g, 13.12 mmol). The resulting solution was allowed to react, with stirring, for 20 minutes while the temperature was maintained at room temperature. The reaction progress was monitored by TLC (ethyl acetate/petroleum ether=1:2). The mixture was concentrated by evaporation under vacuum using a rotary evaporator. The resulting mixture was poured on the water and filtrated; the filtrate cake was washed 3 times with 200 mL of hexane. This resulted in 3.5 g (75%) of tert-butyl 4-(1H-indol-6-yl)piperazine-1-carboxylate as a white solid.

¹H NMR (300 MHz, CDCl₃) δ 1.6 (s, 9H,), 3.1 (s, 4H,), 3.64 (s, 4H,), 6.4 (s, 1H,), 6.9 (d, 2H,), 7.1 (s, 1H), 7.5 (d, 1H), 8.1 (s, 1H). m/z 302 [M+H]⁺

### General Procedure B

### Synthesis of 6-(4-methylpiperazin-1-yl)-1H-indole

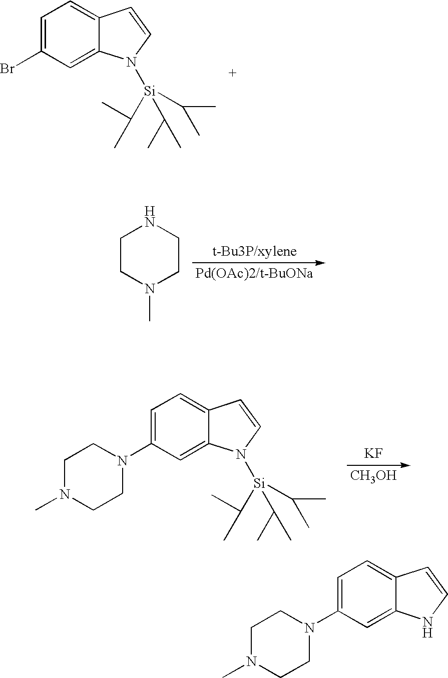

### Synthesis of 6-(4-methylpiperazin-1-yl)-1-(triisopropylsilyl)-1H-indole

Into a 100 mL 3-necked round-bottom flask purged and maintained with an inert atmosphere of nitrogen, was placed a solution of 6-bromo-1-(triisopropylsilyl)-1H-indole (2 g, 5.68 mmol) in Xylene (50 mL). To this was added 1-methylpiperazine (1.95 g, 19.47 mmol). Addition of t-BuONa (760 mg, 7.92 mmol) was next. This was followed by the addition of Pd(OAc)₂ (64 mg, 0.29 mmol). To the mixture was added t-Bu₃P (230 mg, 1.14 mmol). The resulting solution was allowed to react, with stirring, for 30 minutes while the temperature was maintained at 120° C. The reaction progress was monitored by TLC (CH₂Cl₂/MeOH=10:1). The resulting solution was diluted with 100 mL of ethyl acetate. The resulting mixture was washed 2 times with 100 mL of brine. The mixture was dried over Na₂SO₄ and concentrated by evaporation under vacuum using a rotary evaporator. The residue was purified by eluting through a column with a 10:1 CH₂Cl₂/MeOH solvent system. This resulted in 1.7 g (81%) of 6-(4-methylpiperazin-1-yl)-1-(triisopropylsilyl)-1H-indole as light brown oil.

### 2. Synthesis of 6-(4-methylpiperazin-1-yl)-1H-indole

Into a 100 mL round-bottom flask, was placed a solution of 6-(4-methylpiperazin-1-yl)-1-(triisopropylsilyl)-1H-indole (1.7 g, 4.57 mmol) in CH3OH (20 mL). To the mixture was added KF (1.06 g, 18.28 mmol). The resulting solution was allowed to react, with stirring, for 4 hours while the temperature was maintained at room temperature. The reaction progress was monitored by TLC (CH₂Cl₂/MeOH=10:1). The mixture was concentrated by evaporation under vacuum using a rotary evaporator. The resulting solution was diluted with 50 mL of Ethyl acetate. The resulting mixture was washed 3 times with 50 mL of brine. The mixture was dried over MgSO₄. A filtration was performed. The mixture was concentrated by evaporation under vacuum using a rotary evaporator. The residue was purified by eluting through a column with a 50.1-10:1 CH₂Cl₂/MeOH solvent system. This resulted in 0.75 g (76%) of 6-(4-methylpiperazin-1-yl)-1H-indole as a off-white solid.

¹H NMR(300 MHz, DMSO) δ 10.69 (s, 1H), 7.32 (d, 1H), 7.10 (d, 1H), 6.79 (s, 1H), 6.75 (d, 1H), 6.24 (s, 1H), 3.28 (s, 4H), 3.03 (s, 4H), 2.20 (s, 3H). m/z 216 [M+H]⁺

### General Procedure C

### Synthesis of tert-butyl 4-(1H-indazol-6-yl)piperazine-1-carboxylate

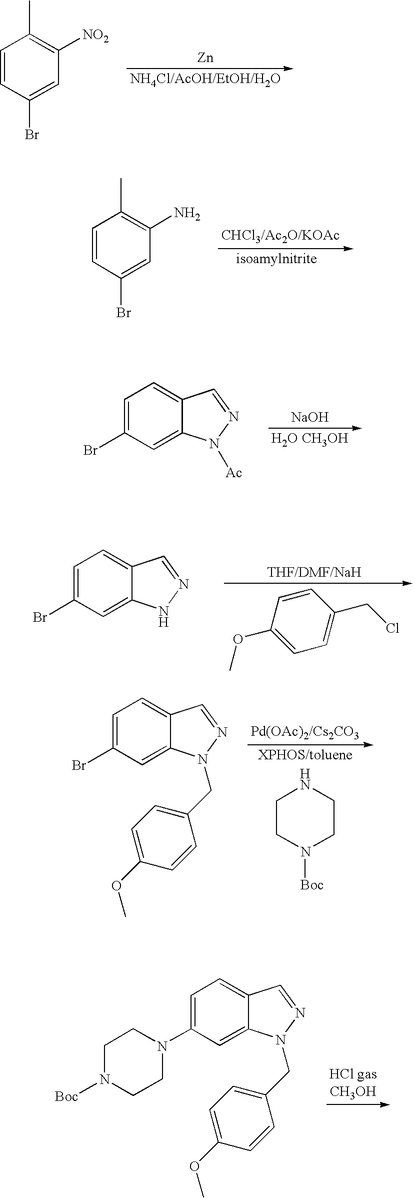

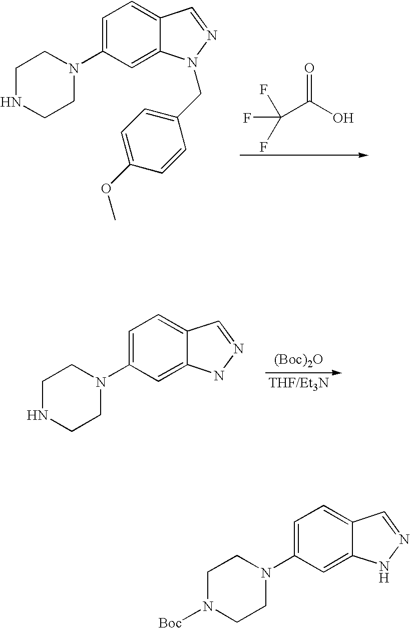

### Synthesis of 5-bromo-2-methylbenzenamine

Into a 1000 mL round-bottom flask, was placed 4-bromo-1-methyl-2-nitrobenzene (40 g, 185.19 mmol). To this was added ammonium chloride (199 g, 3.69 mol). Addition of zinc (120 g, 1.85 mol) was next. This was followed by the addition of a solution of EtOH (500 g) in H₂O (100 mL). To the mixture was added acetic acid (40 mL). The resulting solution was allowed to react, with stirring, for 2 hours while the temperature was maintained at 60° C. in a bath of oil. The reaction progress was monitored by TLC (ethyl acetate/petroleum ether=1:1). A filtration was performed. The filtrate was concentrated by evaporation. A filtration was performed again. This resulted in 20 g (58%) of 5-bromo-2-methylbenzenamine as a brown solid.

### 2. Synthesis of 1-(6-bromo-1H-indazol-1-yl)ethanone

Into a 500 mL round-bottom flask, was placed a solution of 5-bromo-2-methylbenzenamine (25 g, 134.41 mmol) in CHCl3 (60 mL). To the above was added acetic anhydride (27.97 g, 273.95 mmol) dropwise with stirring, while cooling to a temperature of 0° C. over a time period of 1 hour. The resulting solution was allowed to react, with stirring, for 3 hours while the temperature was maintained at 0-5° C. in a bath of H₂O/ice. The reaction progress was monitored by TLC (ethyl acetate/petroleum ether=1:5). Addition of isoamylnitrite (37.35 g, 319.23 mmol) was next. This was followed by the addition of KOAc (4.39 g, 44.80 mmol). To the mixture was added acetic anhydride (47.6 g, 466.21 mmol). The resulting solution was allowed to react, with stirring, overnight while the temperature was maintained at reflux in a bath of oil. The reaction progress was monitored by TLC (ethyl acetate/petroleum ether=1:5). The mixture was concentrated by evaporation under vacuum using a rotary evaporator. Adjustment of the pH to 7 was accomplished by the addition of NaHCO₃ (50%). The resulting solution was extracted three times with 600 mL of ethyl acetate and concentrated by evaporation under vacuum using a rotary evaporator. The residue was purified by eluting through a column with a 1:100 ethyl acetate/petroleum ether solvent system. This resulted in 26 g (81%) of 1-(6-bromo-1H-indazol-1-yl)ethanone as a orange solid.

### 3. Synthesis of 6-bromo-1H-indazole

Into a 500 mL round-bottom flask was placed a solution of 1-(6-bromo-1H-indazol-1-yl)ethanone (7 g, 29.29 mmol) in CH₃OH (200 mL). This was followed by the addition of a solution of NaOH (4.7 g, 117.50 mmol) in H₂O (42.3 my). The resulting solution was allowed to react, with stirring, for 3 hours while the temperature was maintained at reflux in a bath of oil. The reaction progress was monitored by TLC (ethyl acetate/petroleum ether=1:2). The mixture was concentrated by evaporation under vacuum using a rotary evaporator. The residue was dissolved in 40 mL of H₂O. A filtration was performed. The filter cake was washed 1 time with 20 mL of H₂O. This resulted in 5.4 g (93%) of 6-bromo-1H-indazole as a brown solid.

### 4. Synthesis of 1-(4-methoxybenzyl)-6-bromo-1H-indazole

Into a 500 mL round-bottom flask was placed a solution of NaH (2.03 g, 84.58 mmol) in TIE (60 mL). This was followed by the addition of a solution of 6-bromo-1H-indazole (5 g, 25.38 mmol) in THY (70 mL), which was added dropwise with stirring, while cooling to a temperature of 0° C. over a time period of 30 minutes. The resulting solution was allowed to react, with stirring, for 30 minutes while the temperature was maintained at 0° C. in a bath of H₂O/ice. This was followed by the addition of a solution of 1-(chloromethyl)-4-methoxybenzene (5.17 g, 33.01 mmol) in DMF (20 mL), which was added dropwise with stirring, while cooling to a temperature of 0° C. over a time period of 30 minutes. The resulting solution was allowed to react, with stirring, for an additional 4 hours while the temperature was maintained at reflux in a bath of oil. The reaction progress was monitored by TLC (ethyl acetate/petroleum ether=1:2). The residue was dissolved in 70 mL of H₂O. The resulting solution was extracted three times with 300 mL of ethyl acetate and the organic layers combined and dried over Na₂SO₄. A filtration was performed. The filtrate was concentrated by evaporation under vacuum using a rotary evaporator. The residue was purified by eluting through a column with a 1:50 ethyl acetate/petroleum ether solvent system. This resulted in 4.5 g (56%) of 1-(4-methoxybenzyl)-6-bromo-1H-indazole as a white solid.

### 5. Synthesis of tert-butyl 4-(1-(4-methoxybenzyl)-1H-indazol-6-yl)piperazine-1-carboxylate

Into a 150 mL sealed tube purged and maintained with an inert atmosphere of nitrogen, was placed 1-(4-methoxybenzyl)-6-bromo-1H-indazole (4 g, 12.62 mmol). To this was added tert-butyl piperazine-1-carboxylate (7.05 g, 37.85 mmol). Addition of Pd(OAc)₂ (85 mg, 0.38 mmol) was next. This was followed by the addition of Cs₂CO₃ (12.34 g, 37.85 mmol). This was followed by the addition of XPHOS (300 mg, 0.63 mmol). To the mixture was added toluene (60 mL). The resulting solution was allowed to react, with stirring, for 2 days while the temperature was maintained at 100° C. in a bath of oil. The reaction progress was monitored by TLC (ethyl acetate/petroleum ether=1:2). A filtration was performed. The resulting solution was diluted with 70 mL of H₂O. The resulting solution was extracted three times with 500 mL of ethyl acetate and the organic layers combined and dried over Na₂SO₄. A filtration was performed. The filtrate was concentrated by evaporation under vacuum using a rotary evaporator. The residue was purified by eluting through a column with a 1:10 ethyl acetate/petroleum ether solvent system. This resulted in 4.88 g (92%) of tert-butyl 4-(1-(4-methoxybenzyl)-1H-indazol-6-bromo-yl)piperazine-1-carboxylate as a gray green solid.

### 6. Synthesis of 1-(4-methoxybenzyl)-6-(piperazin-1-yl)-1H-indazole

Into a 500 mL 3-necked round-bottom flask was placed a solution of tert-butyl 4-(1-(4-methoxybenzyl)-1H-indazol-6-yl)piperazine-1-carboxylate (2 g, 4.74 mmol) in CH₃OH (150 mL). HCl was put through. The resulting solution was allowed to react, with stirring, for 1.3 hours while the temperature was maintained at room temperature. The reaction progress was monitored by TLC (CH₂Cl₂/MeOH=10:1). The mixture was concentrated by evaporation under vacuum using a rotary evaporator. The resulting mixture was washed 2 times with 50 mL of ether. This resulted in 2 g (crude) of 1-(4-methoxybenzyl)-6-(piperazin-1-yl)-1H-indazole as a brown solid.

### 7. Synthesis of 6-(piperazin-1-yl)-1H-indazole

Into a 150 mL sealed tube was placed a solution of 1-(4-methoxybenzyl)-6-(piperazin-1-yl)-1H-indazole (2 g, 6.21 mmol) in 2,2,2-trifluoroacetic acid (70 mL). To the mixture was added H₂O (2.3 mL). The resulting solution was allowed to react, with stirring, overnight while the temperature was maintained at 105° C. in a bath of oil. The reaction progress was monitored by TLC (CH₂Cl₂/MeOH=5:1). The mixture was concentrated by evaporation under vacuum using a rotary evaporator. The resulting mixture was washed 2 times with 200 mL of ethoxyethane and 2 times with 200 mL of CH₂Cl₂. This resulted in 1.78 g (crude) of 6-(piperazin-1-yl)-1H-indazole as a grey solid.

### 8. Synthesis of tert-butyl 4-(1H-indazol-6-yl)piperazine-1-carboxylate

Into a 150 mL round-bottom flask was placed a solution of 6-(piperazin-1-yl)-1H-indazole (1.67 g, 4.13 mmol) in THE (40 mL). To the mixture was added triethylamine (7 mL). This was followed by the addition of a solution of (Boc)₂O (Boc anhydride ?) (1.26 g, 5.78 mmol) in THF (10 mL), which was added dropwise with stirring, while cooling to a temperature of 0° C. over a time period of 20 minutes. The resulting solution was allowed to react, with stirring, for 30 minutes while the temperature was maintained at room temperature. The reaction progress was monitored by TLC (ethyl acetate/petroleum ether=1:1). The mixture was concentrated by evaporation under vacuum using a rotary evaporator. The residue was purified by eluting through a column with a 1:1 ethyl acetate/petroleum ether solvent system. This resulted in 0.83 g (66%) of tert-butyl 4-(1H-indazol-6-yl)piperazine-1-carboxylate as a grey solid.

¹H NMR (300 MHz, CDCl3) ε1.48(s, 9H), 3.19-3.23(m, 4H), 3.62-3.66(m, 4H), 6.85(s, 1H), 6.95(d, 1H), 7.63(d, 1H), 7.96(s, 1H). m/z 303 [M+H]⁻

### General Procedure D

### Synthesis of 6-(4-methylpiperazin-1-yl)-1H-indazole

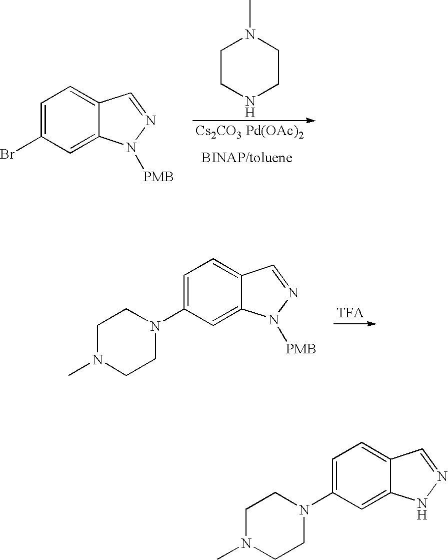

### Synthesis of 1-(4-methoxybenzyl)-6-(4-methylpiperazin-1-yl)-1H-indazole

Into a 150 mL sealed tube was placed a solution of 1-(4-methoxybenzyl)-6-bromo-1H-indazole (4 g, 12.62 mmol) in toluene (60 mL). To this was added 1-methylpiperazine (3.78 g, 37.80 mmol). Addition of Pd(OAc)₂ (284 mg, 1.26 mmol) was next. This was followed by the addition of Cs₂CO₃ (12.34 g, 37.85 mmol). To the mixture was added BINAP (780 mg, 1.25 mmol). After N₂ bubbled, the resulting solution was allowed to react, with stirring, for 20 hours while the temperature was maintained at 100° C. in a bath of oil. The reaction progress was monitored by LC-MS. The mixture was concentrated by evaporation under vacuum using a rotary evaporator. The resulting solution was diluted with 100 mL of H₂O. The resulting solution was extracted three times with 200 mL of ethyl acetate and the organic layers combined and dried over Na₂SO₄. The residue was purified by eluting through a column with a ethyl acetate solvent system. This resulted in 1.7 g (40%) of 1-(4-methoxybenzyl)-6-(4-methylpiperazin-1-yl)-1H-indazole as brown oil.

### 2. Synthesis of 6-(4-methylpiperazin-1-yl)-1H-indazole

Into a 120 mL sealed tube was placed 1-(4-methoxybenzyl)-6-(4-methylpiperazin-1-yl)-1H-indazole (1.2 g, 2.50 mmol). To this was added TFA (20 mL). To the mixture was added H2O (1 mL). The resulting solution was allowed to react, with stirring, overnight while the temperature was maintained at 105° C. in a bath of oil. After adding H₂O, adjustment of the pH to 7-8 was accomplished by the addition of NH₃.H₂O. The resulting solution was extracted six times with 100 mL of ethyl acetate and the organic layers combined and dried over Na₂SO₄ and concentrated by evaporation under vacuum using a rotary evaporator. The residue was purified by eluting through a column with a 20:1 CH₂Cl₂/eOH solvent system. This resulted in 0.5 g (93%) of 6-(4-methylpiperazin-1-yl)-1H-indazole as a brown solid.

¹H NMR (400 MHz, DMSO) δ 2.88 (s, 3H), 3.20 (t, 2H), 3.22 (t, 2H), 3.52 (t, 2H), 3.86 (t, 2H), 6.88 (s, 1H), 6.96 (d, 1H), 7.62 (d, 1H), 7.90 (s, 1H), 12.76 (s, 1H). m/z 217 [M+]⁺.

### General Procedure E

### Synthesis of 2-(1H-indol-6-yl)-octahydropyrrolo[1,2-a]pyrazine

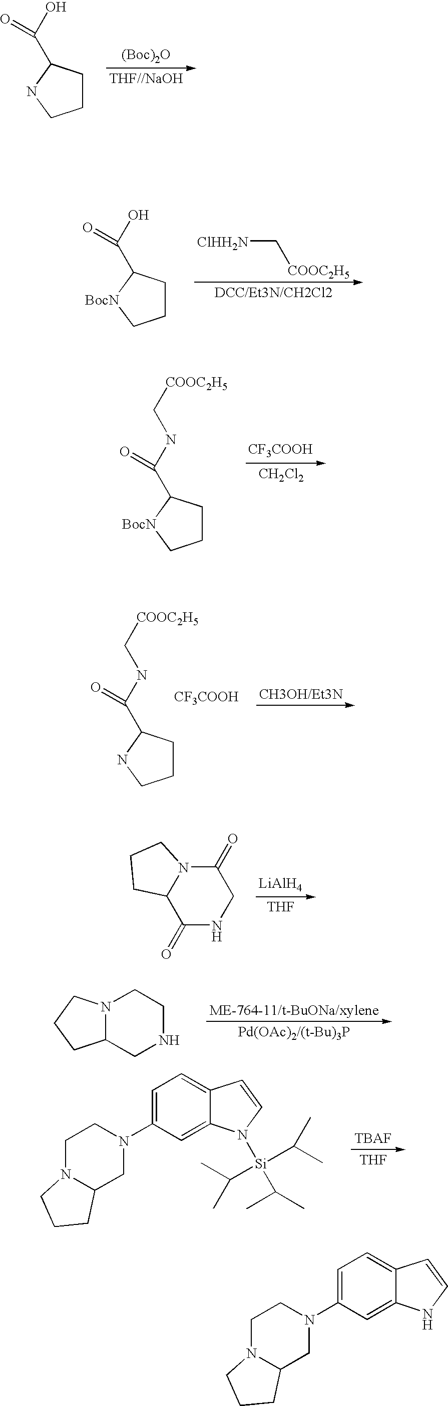

### Synthesis of 1-(tert-butoxycarbonyl)pyrrolidine-2-carboxylic acid

Into a 2000 mL 3-necked round-bottom flask was placed a solution of pyrrolidine-2-carboxylic acid (100 g, 869.57 mmol) in THE (400 mL). This was followed by the addition of a solution of NaOH (69.56 g, 1.74 mol) in H₂O (870 mL). This was followed by the addition of a solution of (Boc)₂O (208.5 g, 956.42 mmol) in THE (300 mL), which was added dropwise with stirring, while cooling to a temperature of 0° C. The resulting solution was allowed to react, with stirring, overnight while the temperature was maintained at room temperature. The reaction progress was monitored by TLC (CH₂Cl₂/MeOH=5;1). The mixture was concentrated by evaporation under vacuum using a rotary evaporator. The resulting solution was extracted two times with 500 mL of ethyl ether and the organic layers combined. Then adjustment of the pH to 3 was accomplished by the addition of HCl (20%) while the temperature was maintained below 10° C. A filtration was performed. The filtrate was extracted 2 times with 500 mL ethyl acetate and the organic layers combined and dried over MgSO₄ and concentrated by evaporation using a rotary evaporator. This resulted in 120 g (58%) of 1-(tert-butoxycarbonyl)pyrrolidine-2-carboxylic acid as a white solid.

### 2. Synthesis of tert-butyl 2-((2-ethoxy-2-oxoethyl)carbamoyl)pyrrolidine-1-carboxylate

Into a 500 mL 3-necked round-bottom flask was placed a solution of ethyl 2-aminoacetate hydrochloride (12.94g, 92.76 mmol) in CH₂Cl₂ (220 mL). To the mixture was added triethylamine (9.36 g, 92.76 mmol). The resulting solution was allowed to react, with stirring, for 20 minutes while the temperature was maintained at 0° C. The mixture was followed by the addition of a solution of 1-(tert-butoxycarbonyl)pyrrolidine-2-carboxylic acid (20 g, 83.53 mmol) in CH₂Cl₂ (100 mL) and N-((cyclohexylimino)methylene)cyclohexanamine (21 g, 101.94 mmol) in CH₂Cl₂ (100 mL), which was added dropwise with stirring. The resulting solution was allowed to react, with stirring, overnight while the temperature was maintained at room temperature. The reaction progress was monitored by TLC(CH₂Cl₂/MeOH=10:1). A filtration was performed. The filtrate was washed one time with 20 mL of NaHCO₃ (sat.) and one time with 20 mL of NaCl (sat.) and dried over Na₂SO₄ and concentrated by evaporation under vacuum using a rotary evaporator. This resulted in 32 g (crude) of tert-butyl 2-((2-ethoxy-2-oxoethyl)carbamoyl)pyrrolidine-1-carboxylate as a light yellow liquid.

### 3. Synthesis of De-Boc Compound

Into a 50 mL round-bottom flask, was placed a solution of tert-butyl 2-((2-ethoxy-2-oxoethyl)carbamoyl)pyrrolidine-1-carboxylate (1.0 g, 3.00 mmol) in CF3COOH (10 mL). The resulting solution was allowed to react, with stirring, for 1.5 hours while the temperature was maintained at room temperature. The reaction progress was monitored by TLC (Ethyl acetate/PE=1:1). The mixture was concentrated by evaporation under vacuum using a rotary evaporator. This resulted in 1.5 g (crude) of de-Boc compound as a light yellow liquid.

### 4. Synthesis of Hexahydropyrrolo[1,2-a]pyrazine-1,4-dione

Into a 500 mL round-bottom flask, was placed a solution of PH-ME-764-3 (80.3 g, 179.01 mmol) in CH₃OH (300 mL). To the mixture was added triethylamine (74 g, 732.67 mmol). The resulting solution was allowed to react, with stirring, overnight while the temperature was maintained at reflux in a bath of oil. The reaction progress was monitored by TLC(CH₂Cl₂/MeOH=10:1). The mixture was concentrated by evaporation under vacuum using a rotary evaporator. The final product was purified by recrystallization from propan-2-ol. This resulted in 22.62 g (57%) of hexahydropyrrolo[1,2-a]pyrazine-1,4-dione as a white solid.

### 5. Synthesis of octahydropyrrolo[1,2-a]pyrazine

Into a 1000 mL round-bottom flask was placed a solution of hexahydropyrrolo[1,2-a]pyrazine-1,4-dione (22.62 g, 117.51 mmol) in THF (600 mL). To the above was added LiAlH4 (22.62 g, 595.26 mmol) in several batches, while cooling to a temperature of 0° C. over a time period of 1 hour. The resulting solution was allowed to react, with stirring, for 30 minutes while the temperature was maintained at room temperature. The reaction progress was monitored by TLC (CH₂Cl₂/MeOH=5:1). The resulting solution was allowed to react, with stirring, overnight while the temperature was maintained at reflux. The mixture was cooled to 0° C. The reaction mixture was then quenched by the adding 22.3 mL of H₂O, 22.3 mL of 15% NaOH and 66.96 mL of H₂O. The mixture was stirred for 1 hour. A filtration was performed. The filter cake was washed with THF. The mixture was dried over Na₂SO₄ and concentrated by evaporation under vacuum using a rotary evaporator. The residue was dissolved in 20 mL of CH₂Cl₂. A filtration was performed. The filtrate was concentrated by evaporation under vacuum using a rotary evaporator. This resulted in 9.0 g (61%) of octahydropyrrolo[1,2-a]pyrazine as a light yellow liquid.

### 6. Synthesis of 6-bromo-1-(triisopropylsilyl)-1H-indole

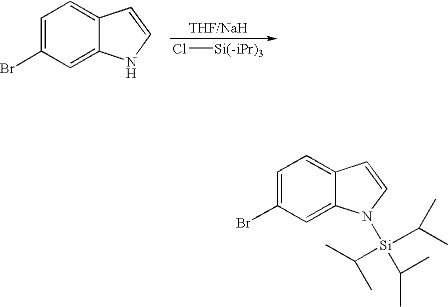

Into a 50 mL 3-necked round-bottom flask was placed TVF (10 mL). To the above was added NaH (250 mg, 6.25 mmol) in several batches, while cooling to a temperature of 0° C. To the above was added 6-bromo-1H-indole (1 g, 5.16 mmol) in several batches, while cooling to a temperature of 0° C. The mixture was stirred for 20 minutes at that temperature. This was followed by the addition of a solution of chlorotriisopropylsilane (1.11 g, 5.75 mmol) in THF (5 mL), which was added dropwise with stirring, while cooling to a temperature of 0° C. The resulting solution was allowed to react, with stirring, for 20 minutes while the temperature was maintained at 0° C. in a bath of H₂O/ice. The reaction progress was monitored by TLC (ethyl acetate/petroleum ether=1:2). The mixture was concentrated by evaporation under vacuum using a rotary evaporator. The residue was dissolved in ethyl acetate. The resulting mixture was washed 5 times with 30 mL of brine. The mixture was dried over MgSO₄ and concentrated by evaporation under vacuum using a rotary evaporator. This resulted in 1.2 g (59%) of 6-bromo-1-(triisopropylsilyl)-1H-indole as brown oil.

### 7. Synthesis of 2-(1-(triisopropylsilyl)-1H-indol-6-yl)-octahydropyrrolo[1,2-a]pyrazine

Into a 50 mL 3-necked round-bottom flask purged and maintained with an inert atmosphere of nitrogen was placed a solution of 6-bromo-1-(triisopropylsilyl)-1H-indole (500 mg, 1.35 mmol) in xylene (10 mL). To this was added octahydropyrrolo[1,2-a]pyrazine (1.075 g, 7.10 mmol). Addition of t-BuONa (0.19 mg) was next. This was followed by the addition of Pd(OAc)₂ (12.75 mg, 0.06 mmol). To the mixture was added tri-tert-butylphosphine (45.9 mg, 0.22 mmol). The resulting solution was allowed to react, with stirring, two hours while the temperature was maintained at 120° C. in a bath of oil. The reaction progress was monitored by TLC (ethyl acatate/petroleum ether=1:5). The reaction mixture was then quenched by the adding 100 mL of Ethyl acetate. The resulting mixture was washed 3 times with 50 mL of NaCl(sat.). The mixture was dried over MgSO₄ and concentrated by evaporation under vacuum using a rotary evaporator. The residue was purified by eluting through a column with a 100:1 CH₂Cl₂/MeOH solvent system. This resulted in 210 mg (31%) of 2-(1-(triisopropylsilyl)-1H-indol-6-yl)-octahydropyrrolo[1,2-a]pyrazine as a light yellow liquid.

### 8. Synthesis of 2-(1H-indol-6-yl)-octahydropyrrolo[1,2-a]pyrazine

Into a 100 mL round-bottom flask was placed a solution of 2-(1-(triisopropylsilyl)-1H-indol-6-yl)-octahydropyrrolo[1,2-a]pyrazine (1.5 g, 3.02 mmol) in THF (20 mL). This was followed by the addition of a solution of TBAF (990 mg, 3.79 mmol) in THF (30 mL). The resulting solution was allowed to react, with stirring, for 30 minutes while the temperature was maintained at RT ° C. The reaction progress was monitored by TLC (CH₂Cl₂/MeOH=10:1). The mixture was concentrated by evaporation under vacuum using a rotary evaporator. The residue was dissolved in 100 mL of Ethyl acetate. The resulting mixture was washed 3 times with 50 mL of brine. The mixture was dried over MgSO₄ and concentrated by evaporation under vacuum using a rotary evaporator. The residue was purified by eluting through a column with a 100:1 CH₂Cl₂/MeOH solvent system. This resulted in 0.39 g (44%) of 2-(1H-indol-6-yl)-octahydropyrrolo[1,2-a]pyrazine as a grey solid.

¹H NMR (400 MHz, CDCl₃) δ 8.02 (m, 1H), 7.49 (d, 1H), 7.07 (d, 1H), 6.90 (m, 2H), 6.43 (s, 1H), 3.71 (d, 1H), 3.68 (d, 1H), 3.18 (m, 2H), 3.10 (m, 1H), 2.55 (m, 2H), 2.20 (m, 2H), 1.92 (m, 4H). m/z 242 [M+H]⁺

### General Procedure F

### Synthesis of 2-(1H-indol-6-yl)-octahydro-1H-pyrido[1,2-a]pyrazine

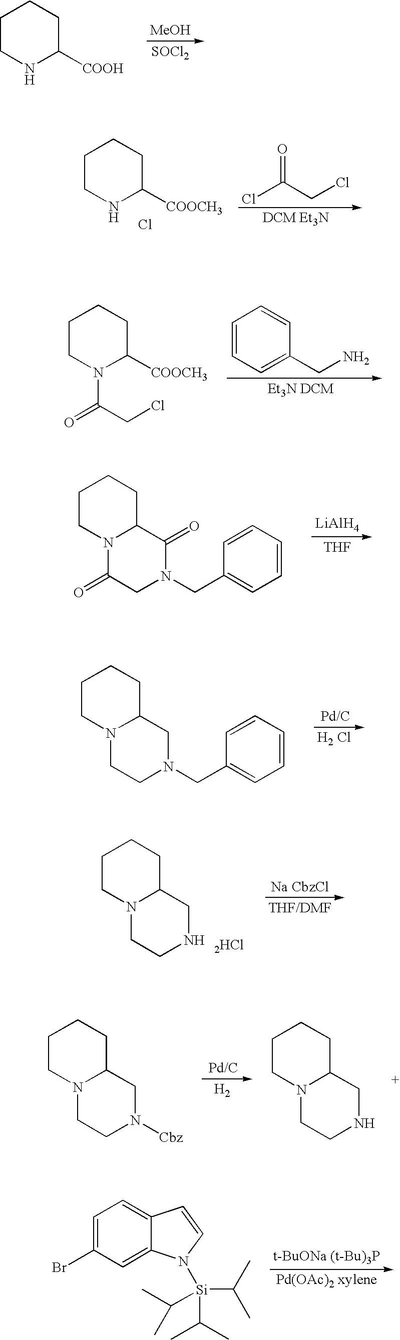

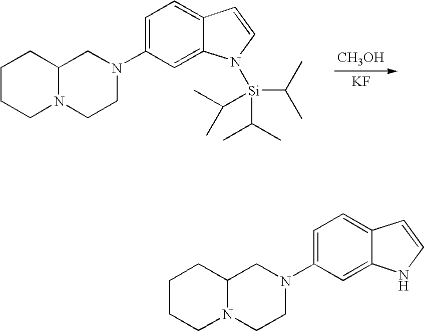

### Synthesis of methyl piperidine-2-carboxylate hydrochloride

Into a 3000 mL 3-necked round-bottom flask was placed CH₃OH (1300 mL). The temperature was cooled to −30° C. To the above was added SOCl₂ (280 mL) dropwise with stirring, while the temperature was maintained at −30° C. To the above was added piperidine-2-carboxylic acid (100 g, 766.49 mmol) in several batches, while cooling to a temperature of 0° C. The resulting solution was allowed to react, with stirring, for 15 hours while the temperature was maintained at room temperature. The reaction progress was monitored by TLC (CH₃OH adding several drops of NH₃ H₂O). The mixture was concentrated by evaporation under vacuum using a rotary evaporator. To this was added toluene (100 mL), the temperature was maintained at reflux. The mixture was concentrated by evaporation under vacuum using a rotary evaporator. The steps of adding toluene and concentrated were repeated twice. This resulted in 137 g (98%) of methyl piperidine-2-carboxylate hydrochloride as a white solid.

### 2. Synthesis of methyl 1-(2-chloroacetyl)piperidine-2-carboxylate

Into a 3000 mL 3-necked round-bottom flask, was placed CH₂Cl₂ (1500 mL). To this was added Et₃N (154.35 g, 1.53 mol). To the mixture was added methyl piperidine-2-carboxylate hydrochloride (137 g, 739.76 mmol). Then the temperature was cooled to 0° C. This was followed by the addition of a solution of 2-chloroacetyl chloride (86.13 g, 762.62 mmol) in DCM (500 mL), which was added dropwise with stirring, while the temperature maintained at 0° C. The resulting solution was allowed to react, with stirring, for 2 hours while the temperature was maintained at 0° C. in a bath of H₂O/ice. The reaction progress was monitored by TLC (CH₂Cl₂/MeOH=10:1, adding one drop of NH₃.H₂O). The resulting mixture was washed 1 time with 200 mL of H₂O, 1 time with 250 mL of saturated NaHCO₃ solution and 1 time with 200 mL of saturated NaCl solution. The mixture was dried over Na₂SO₄. A filtration was performed. This resulted in methyl 1-(2-chloroacetyl)piperidine-2-carboxylate and the product was directly used by the next reaction.

### 3. Synthesis of 2-benzyl-hexahydro-6H-pyrido[1,2-a]pyrazine-1,4-dione

Into a 5000 mL 3-necked round-bottom flask was placed DCM (1000 mL). To this was added phenylmethanamine (81.72 g, 762.67 mmol). Addition of Et3N (77.17 g, 762.62 mmol) was next. This was followed by the addition of a solution of methyl 1-(2-chloroacetyl) piperidine-2-carboxylate (167.53 g) in CH₂Cl₂ (2500 mL). The resulting solution was allowed to react, with stirring, for 48 hours while the temperature was maintained at room temperature. The reaction progress was monitored by TLC (ethyl acetate/petroleum ether=2:1). The resulting mixture was washed 2 times with 100 mL of H₂O and 2 times with 100 mL of the saturated NaHCO₃ solution. The mixture was dried over Na₂SO₄. A filtration was performed. The filtrate was concentrated by evaporation under vacuum using a rotary evaporator. The final product was purified by recrystallization from CH₂Cl₂ and hexane. This resulted in 130 g (75%) of 2-benzyl-hexahydro-6H-pyrido[1,2-a]pyrazine-1,4-dione as a white solid.

### 4. Synthesis of 2-benzyl-octahydro-1H-pyrido[1,2-a]pyrazine

Into a 5000 mL 3-necked round-bottom flask was placed a solution of 2-benzyl-hexahydro-6H-pyrido[1,2-a]pyrazine-1,4-dione (96 g, 353.05 mmol) in THF (2000 mL) while cooling to 0° C. To the above was added LiAlH (91.67 g, 2.42 mol) in several batches. The resulting solution was allowed to react with stirring, for 3 hours while the temperature was maintained at reflux in a bath of oil. The reaction progress was monitored by TLC(CH₂Cl₂MeOH=10:1). The mixture was added 92 mL H₂O, 276 mL NaOH solution and 92 mL H₂O. A filtration was performed. The mixture was dried over MgSO₄ and concentrated by evaporation under vacuum using a rotary evaporator. The residue was purified by eluting through a column with a 1:1 ethyl acetate/petroleum ether solvent system. This resulted in 60 g (70%) of 2-benzyl-octahydro-1H-pyrido[1,2-a]pyrazine as a yellow solid.

### 5. Synthesis of octahydro-1H-pyrido[1,2-a]pyrazine dihydrochloride

Into a 1000 mL round-bottom flask was placed a solution of 2-benzyl-octahydro-1H-pyrido[1,2-a]pyrazine (1.5 g, 6.38 mmol) in CH₃OH (300 mL). To the mixture was added Pd/C (3 g). To the above was added HCl (1.94 mL) dropwise with stirring. Then hydrogen was passed through the mixture. The resulting solution was allowed to react, with stirring, for 5 hours while the temperature was maintained at room temperature. The reaction progress was monitored by TLC (CH₂Cl₂/MeOH=10:1). A filtration was performed. The filtrate was concentrated by evaporation under vacuum using a rotary evaporator. This resulted in 1.37 g (96%) of octahydro-1H-pyrido[1,2-a]pyrazine dihydrochloride as a white solid.

### 6. Synthesis of benzyl hexahydro-1H-pyrido[1,2-a]pyrazine-2(6H)-carboxylate

Into a 1000 mL round-bottom flask purged and maintained with an inert atmosphere of argon was placed NaI (11.82 g, 492.50 mmol). To this was added THF (100 mL). To the mixture was added DMF (2 mL). This was followed by the addition of a solution of octahydro-1H-pyrido[1,2-a]pyrazine dihydrochloride (18 g, 80.23 mmol) in TVF (50 mL), which was added dropwise with stirring, while cooling to a temperature of 0° C. over a time period of 10 minutes. This was followed by the addition of a solution of benzyl chloroformate (28.79 g, 168.86 mmol) in THF (50 mL), which was added dropwise with stirring, while cooling to a temperature of 0° C. over a time period of 30 minutes. The resulting solution was allowed to react, with stirring, for 2 hours while the temperature was maintained at rt ° C. The reaction progress was monitored by TLC (CH₂Cl₂/MeOH=10:1). A filtration was performed. The filter cake was washed 1 time with 60 mL of H₂O. The resulting solution was extracted three times with 50 mL of ethyl acetate dried over MgSO₄. The residue was purified by eluting through a column with a 50:1 CH₂Cl₂/MeOH solvent system. This resulted in 20 g (87%) of benzyl hexahydro-1H-pyrido[1,2-a]pyrazine-2(6H)-carboxylate as yellow oil.

### 7. Synthesis of octahydro-1H-pyrido[1,2-a]pyrazine

Into a 500 mL round-bottom flask was added benzyl hexahydro-1H-pyrido[1,2-a]pyrazine-2(6H)-carboxylate (8.5 g, 29.45 mmol). To this was added MeOH (200 mL). To the mixture was added Pd/C (15 g) followed by hydrogen. The resulting solution was allowed to react, with stirring, for 4 hours while the temperature was maintained at r.t ° C. The reaction progress was monitored by TLC (CH₂Cl₂/MeOH=10:1). A filtration was performed. The filtrate was concentrated by evaporation under vacuum using a rotary evaporator. This resulted in 2.5 g (60%) of octahydro-1H-pyrido[1,2-a]pyrazine as a white solid.

### 8. Synthesis of 2-(1-(triisopropylsityl)-1H-indol-6-yl)-octahydro-1H-pyrido[1,2-a]pyrazine

Into a 100 mL 3-necked round-bottom flask purged and maintained with an inert atmosphere of nitrogen was placed 6-bromo-1-(triisopropylsilyl)-1H-indole (2 g, 5.40 mmol). To this was added octahydro-1H-pyrido[1,2-a]pyrazine (2.1 g, 14.25 mmol). Addition of t-BuONa (2 g, 20.83 mmol) was next. This was followed by the addition of (t-Bu)3P (200 mg, 0.99 mmol). This was followed by the addition of Pd(OAc)2 (50 mg, 0.22 mmol). To the mixture was added xylene (30 mL). The resulting solution was allowed to react, with stirring, for 2 hours while the temperature was maintained at 110° C. in a bath of oil. The reaction progress was monitored by TLC (CH₂Cl₂MeOH=10:1). The resulting mixture was washed 1 time with 30 mL of H₂O. The resulting solution was extracted three times with 100 mL of ethyl acetate and the organic layers combined. The resulting mixture was washed 1 time with 10 mL of NaCl(sat.). The mixture was dried over Na₂SO₄ and concentrated by evaporation under vacuum using a rotary evaporator. This resulted in 2.15 g (80%) of 2-(1-(triisopropylsilyl)-1H-indol-6-yl)-octahydro-1H-pyrido[1,2-a]pyrazine as red oil.

### 9. Synthesis of 2-(1H-indol-6-yl)-octahydro-1H-pyrido[1,2-a]pyrazine

Into a 100 mL round-bottom flask was placed 2-(1-(triisopropylsilyl)-1H-indol-6-yl)-octahydro-1H-pyrido[1,2-a]pyrazine (2.5 g, 5.78 mmol). To this was added CH3OH (20 mL). To the mixture was added KF (1.4 g, 24.14 mmol). The resulting solution was allowed to react, with stirring, for 4 hours while the temperature was maintained at room temperature. The reaction progress was monitored by TLC (CH₂Cl₂MeOH=10:1). The mixture was concentrated by evaporation under vacuum using a rotary evaporator. The resulting mixture was washed 1 time with 15 mL of H₂O. The resulting solution was extracted three times with 50 mL of CH₂Cl₂ dried over Na₂SO₄ and concentrated by evaporation under vacuum using a rotary evaporator. The residue was purified by eluting through a column with a 50:1 CH₂Cl₂/MeOH solvent system. This resulted in 0.9 g (60%) of 2-(1H-indol-6-yl)-octahydro-1H-pyrido[1,2-a]pyrazine as a off-white solid.

¹H-NMR (400 Hz, CDCl3) δ 8.15(m, 1H), 7.51(d, 1H), 7.06(d, 1H), 6.89(d, 1H), 6.87(d, 1H), 6.44(s, 1H), 3.5(d, 1H), 3.4(d, 1H), 2.96(d, 1H), 2.90(d, 1H), 2.86(d, 1H), 2.58(t, 1H), 2.49(m, 1H), 2.17(t, 1H), 2.12(t, 1H), 1.8(m, 1H), 1.68(m, 1H), 1.62(m, 1H), 1.60(m, 1H), 1.35(m, 1H), 1.33(m, 1H). m/z 256 [M+H]⁺

### General Procedure G

### Synthesis of 6-(Hexahydropyrrolo[1,2-a]pyrazin-2(1H)-yl)-1H-indazole

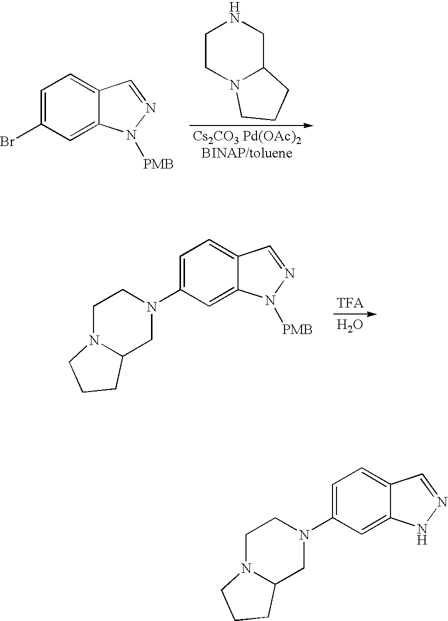

### Synthesis of 1-(4-methoxybenzyl)-6-(hexahydropyrrolo[1,2-a]pyrazin-2(1H)-yl)-1H-indazole

Into a 150 mL sealed tube was placed 1-(4-methoxybenzyl)-6-bromo-1H-indazole (5 g, 15.77 mmol). To this was added octahydropyrrolo[1,2-a]pyrazine (5.96 g, 47.30 mmol). Addition of Pd(OAc)2 (355 mg, 1.58 mmol) was next. This was followed by the addition of Cs₂CO₃ (15.4 g, 47.24 mmol). To the mixture was added toluene (70 mL). After N2 bubbled, this was followed by the addition of BINAP (982 mg, 1.58 mmol). After N2 bubbled, the resulting solution was allowed to react, with stirring, for 21 hours while the temperature was maintained at 100° C. in a bath of oil. The reaction progress was monitored by TLC (CH₂Cl₂/MeOH=10:1). The mixture was concentrated by evaporation under vacuum using a rotary evaporator. The residue was dissolved in 200 mL of H₂O. The resulting solution was extracted three times with 300 mL of ethyl acetate and the organic layers combined and dried over Na₂SO₄. The residue was purified by eluting through a column with a 1:2 ethyl acetate/petroleum ether solvent system. This resulted in 1.9 g (33%) of 1-(4-methoxybenzyl)-6-(hexahydropyrrolo[1,2-a]pyrazin-2(1H)-yl)-1H-indazole as brown oil.

### 2. Synthesis of 6-(hexahydropyrrolo[1,2-a]pyrazin-2(1H)-yl)-1H-indazole

Into a 120 mL sealed tube was placed 1-(4-methoxybenzyl)-6-(hexahydropyrrolo[1,2-a]pyrazin-2 (1H)-yl)-1H-indazole (2.5 g, 6.91 mmol). To this was added TFA (30 mL). To the mixture was added H₂O (1 mL). The resulting solution was allowed to react, with stirring, for 24 hours while the temperature was maintained at 105° C. in a bath of oil. The reaction progress was monitored by TLC (CH₂Cl₂/MeOH=10:1). After adding H₂O (100 mL), adjustment of the pH to 7-8 was accomplished by the addition of NH₃. H₂O. The resulting solution was extracted three times with 300 mL of CH₂Cl₂ and the organic layers combined and dried over Na₂SO₄. The residue was purified by eluting through a column with a 1:20 MeOH: CH₂Cl₂ solvent system. The resulting mixture was washed with CH₂Cl₂. A filtration was performed and the filtrated cake collected. This resulted in 1.1 g (66%) of 6-(hexahydropyrrolo[1,2-a]pyrazin-2(1H)-yl)-1H-indazole as a white solid.

¹H NMR (400 MHz, DMSO-d₆) δ 1.37 (t, 1H), 1.70 (t, 2H), 1.81 (t, 1H), 2.07 (t, 2H), 2.25 (t, 1H), 2.43 (t, 1H), 2.76 (t, 1H), 3.03 (t, 2H), 3.62 (d, 1H), 3.77 (d, 1H), 6.76 (s, 1H), 6.91 (d, 1H), 7.52 (d, 1H), 7.82 (s, 1H), 12.57 (s, 1H). m/z 243 [M+H]⁺

### General Procedure H

### Synthesis of tert-butyl 4-(3-ethyl-1H-indazol-6-yl)piperazine-1-carboxylate

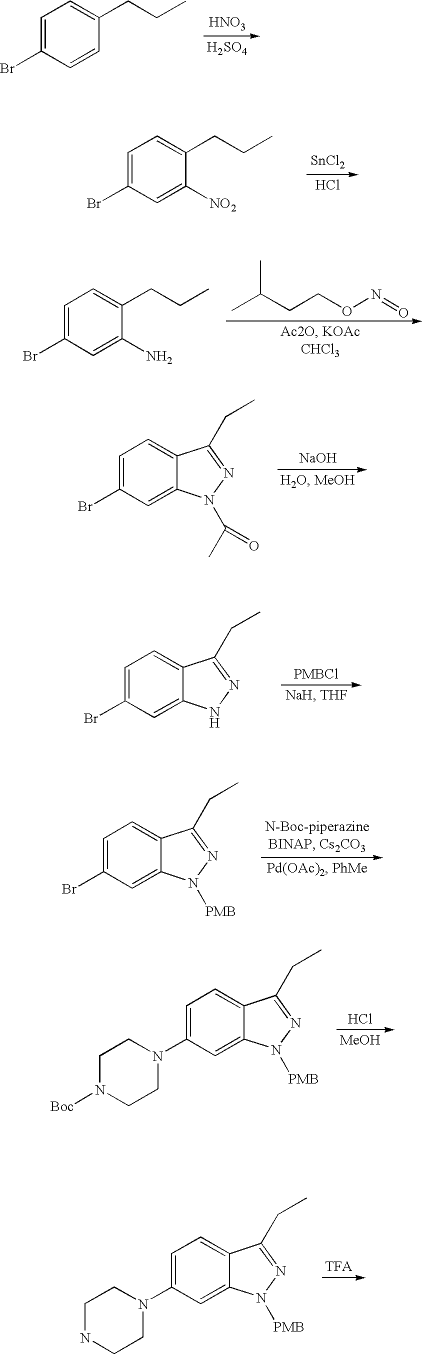

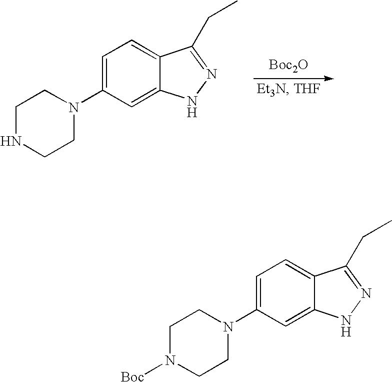

### Synthesis of 4-bromo-2-nitro-1-propylbenzene

Into a 500 mL 3-necked round-bottom flask was placed a solution of 1-bromo-4-propylbenzene (70 g, 351.76 mmol) in H₂SO₄ (70 mL). To the mixture was added HNO3/H₂SO₄ (24.5/35 g, 369.44 mmol). The resulting solution was allowed to react, with stirring, for 20 minutes while the temperature was maintained at −30-20° C. The reaction progress was monitored by TLC (ethyl acetate/petroleum ether=1.10). The reaction mixture was then quenched by the adding 500 mL of H₂O/ice. The resulting solution was extracted three times with 2000 mL of ethyl acetate and the organic layers combined. The resulting mixture was washed 3 times with NaHCO₃ and 2 times with H₂O. The mixture was dried over Na₂SO₄ and concentrated by evaporation under vacuum using a rotary evaporator. The residue was purified by eluting through a column with a pure petroleum ether solvent system. This resulted in 35.1 g (41%) of 4-bromo-2-nitro-1-propylbenzene as yellow oil.

### 2. Synthesis of 5-bromo-2-propylbenzenamine

Into a 1000 mL 3-necked round-bottom flask was placed a solution of 4-bromo-2-nitro-1-propylbenzene (45 g, 184.43 mmol) in EtOH/H₂O (213.75/11.25 mL). This was followed by the addition of a solution of SnCl2 (135 g, 714.29 mmol) in HCl (202.5 mL). The resulting solution was allowed to react, with stirring, for 1 hour while the temperature was maintained at room temperature in a bath of H₂O. The reaction progress was monitored by TLC (ethyl acetate/petroleum ether-1:10). The reaction mixture was then quenched by the adding 200 mL of H₂O/ice. Adjustment of the pH to 8 was accomplished by the addition of NaOH. The resulting solution was extracted 2 times with 1000 mL of ethyl ether and the organic layers combined. The resulting mixture was washed 1 time with H₂O. The mixture was dried over Na₂SO₄ and concentrated by evaporation under vacuum using a rotary evaporator. This resulted in 37.9 g (96%) of 5-bromo-2-propylbenzenamine as red oil.

### 3. Synthesis of 1-(6-bromo-3-ethyl-1H-indazol-1-yl)ethanone

Into a 1000 mL 3-necked round-bottom flask was placed a solution of 5-bromo-2-propylbenzenamine (37 g, 172.82 mmol) in CHCl3 (350 mL). To the above was added acetic anhydride (44 g, 430.95 mmol) dropwise with stirring, while cooling to a temperature of 0° C. The resulting solution was allowed to react, with stirring, for 1 hour at room temperature. Addition of Isoamyl Nitrite (81 g, 691.72 mmol) was next. This was followed by the addition of KOAc (6.8 g, 69.32 mmol). To the mixture was added acetic anhydride (53 g, 519.10 mmol). The resulting solution was allowed to react, with stirring, overnight while the temperature was maintained at reflux. The reaction progress was monitored by TLC (ethyl acetate/petroleum ether=1:5). The mixture was concentrated by evaporation under vacuum using a rotary evaporator. This resulted in 73 g (crude) of 1-(6-bromo-3-ethyl-1H-indazol-1-yl)ethanone as a red solid.

### 4. Synthesis of 6-bromo-3-ethyl-1H-indazole

Into a 1000 mL round-bottom flask was placed a solution of 1-(6-bromo-3-ethyl-1H-indazol-1-yl)ethanone (46 g, 172.21 mmol) in MeOH/H₂O (400/100 mL). To the mixture was added NaOH (27.7 g, 690 mmol). The resulting solution was allowed to react, with stirring, for 30 minutes while the temperature was maintained at reflux. The reaction progress was monitored by TLC (ethyl acetate/petroleum ether=1:2). The mixture was concentrated by evaporation. The resulting solution was diluted with 500 mL of ethyl acetate. The resulting mixture was washed 2 times with 500 mL of brine. The mixture was dried over Na₂SO₄. The residue was purified by eluting through a column with a 1:10 ethyl acetate/petroleum ether solvent system. This resulted in 27 g (44%) of 6-bromo-3-ethyl-1H-indazole as a yellow solid.

### 5. Synthesis of 1-(4-methoxybenzyl)-6-bromo-3-ethyl-1H-indazole

Into a 500 mL 3-necked round-bottom flask was placed a solution of NaH (3.2 g, 80.00 mmol) in THF (100 mL). The temperature was cooled to 0° C. This was followed by the addition of a solution of 6-bromo-3-ethyl-1H-indazole (15 g, 66.67 mmol) in THF (100 mL), which was added dropwise with stirring, while cooling to a temperature of 0° C. The resulting solution was allowed to react, with stirring, for 30 minutes while the temperature was maintained at 0° C. This was followed by the addition of a solution of 1-(chloromethyl)-4-methoxybenzene (13.7 g, 87.26 mmol) in DMF (70 mL), which was added dropwise with stirring, while cooling to a temperature of 0° C. The resulting solution was allowed to react, with stirring, for an additional 2 hours while the temperature was maintained at reflux in a bath of oil. The reaction progress was monitored by TLC (ethyl acetate/petroleum ether=1:2). The mixture was concentrated by evaporation under vacuum using a rotary evaporator The residue was dissolved in 400 mL of ethyl acetate. The resulting mixture was washed 3 times with 150 mL of H₂O. The mixture was dried over MgSO₄ and concentrated by evaporation under vacuum using a rotary evaporator. This resulted in 26.0 g (crude) of 1-(4-methoxybenzyl)-6-bromo-3-ethyl-1H-indazole as yellow oil.

### 6. Synthesis of tert-butyl 4-(1-(4-methoxybenzyl)-3-ethyl-1H-indazol-6-yl)piperazine-1-carboxylate

Into a 150 mL sealed tube purged and maintained with an inert atmosphere of nitrogen was placed a solution of 1-(4-methoxybenzyl)-6-bromo-3-ethyl-1H-indazole (5 g, 14.49 mmol) in toluene (50 mL). To this was added C₉H₁₈N₂O₂ (8.1 g, 43.55 mmol). Addition of Pd(OAc)2 (330 mg, 1.47 mmol) was next. This was followed by the addition of Cs₂CO₃ (14.2 g, 43.56 mmol). To the mixture was added BINAP (270 mg, 0.43 mmol). The resulting solution was allowed to react, with stirring, overnight while the temperature was maintained at 120° C. in a bath of oil. The reaction progress was monitored by TLC (ethyl acetate/petroleum ether=1:2). The mixture was concentrated by evaporation. The resulting solution was extracted 3 times with 500 mL of ethyl acetate and the organic layers combined. The resulting mixture was washed 3 times with 500 mL of NaCl. The mixture was concentrated by evaporation. The residue was purified by eluting through a column with a 1:5 ethyl acetate/petroleum ether solvent system. This resulted in 5.8 g (89%) of tert-butyl 4-(1-(4-methoxybenzyl)-3-ethyl-1H-indazol-6-yl)piperazine-1-carboxylate as red oil.

### 7. Synthesis of 3-ethyl-6-piperazin-1-yl-1H-indazole

Into a 500 mL 3-necked round-bottom flask was placed a solution of tert-butyl 4-(1-(4-methoxybenzyl)-3-ethyl-1H-indazol-6-yl)piperazine-1-carboxylate (5.8 g, 12.86 mmol) in MeOH (150 mL). To the mixture was added HCl(g). The resulting solution was allowed to react, with stirring, for 1 hour while the temperature was maintained at room temperature. The reaction progress was monitored by TLC (CH₂Cl₂/MeOH=5:1). The mixture was concentrated by evaporation under vacuum using a rotary evaporator.

Into a 150 mL sealed tube was placed a solution of 1-(4-methoxybenzyl)-3-ethyl-6-(piperazin-1-yl)-1H-indazole (4 g, 11.43 mmol) in TFA/H₂O (60/5 mL). The resulting solution was allowed to react, with stirring, for 40 hours while the temperature was maintained at 105° C. in a bath of oil. The reaction progress was monitored by TLC (CH2Cl2/MeOH=5:1).The mixture was concentrated by evaporation under vacuum using a rotary evaporator.

### 8. Synthesis of tert-butyl 4-(3-ethyl-1H-indazol-6-yl)piperazine-1-carboxylate

Into a 500 mL 3-necked round-bottom flask was placed a solution of 3-ethyl-6-(piperazin-1-yl)-1H-indazole (2.4 g, 10.43 mmol) in THF (200 mL). To the above was added Et3N (10 mL) dropwise with stirring, while cooling to a temperature of 0° C. over a time period of 10 minutes. This was followed by the addition of a solution of (Boc)20 (3.18 g, 14.59 mmol) in THF (40 mL), which was added dropwise with stirring, while cooling to a temperature of 0° C. The resulting solution was allowed to react, with stirring, for 30 minutes while the temperature was maintained at room temperature. The reaction progress was monitored by TLC (CH2Cl2/petroleum ether=5:1). The mixture was concentrated by evaporation under vacuum using a rotary evaporator. The resulting solution was diluted with of Ethyl acetate. The resulting mixture was washed 3 times with NaCl solution. The mixture was dried over Na₂SO₄. The residue was purified by eluting through a column with a 1:2 Ethyl acetate/PE solvent system. This resulted in 2.55 g (73%) of tert-butyl 4-(3-ethyl-1H-indazol-6-yl)piperazine-1-carboxylate as a yellow solid.

¹H NMR(300 MHz, CDCl3) δ: 7.6(d, 1H), 6.9(d, 1H), 6.7(s, 1H), 3.6(m, 4H), 3.2(m, 4H), 2.95(m, 2H), 1.5 (s, 9H), 1.41 (m, 3H). m/z 331 [M+H]⁺

### General Procedure I

### Synthesis of 3-ethyl-6-(4-methylpiperazin-1-yl)-1H-indazole

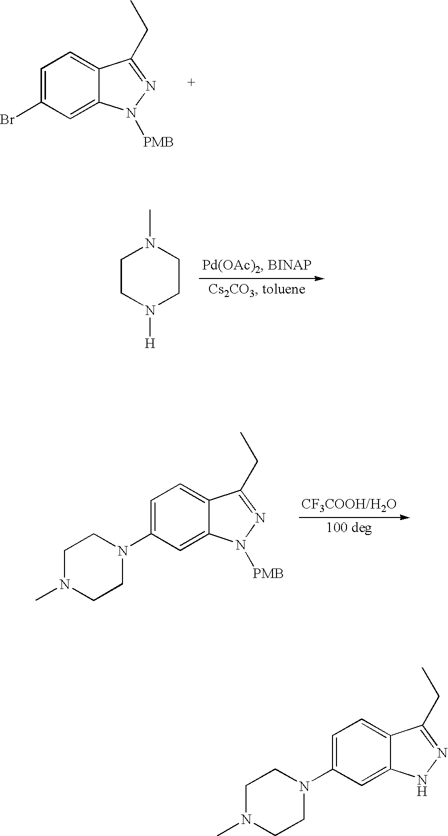

### Synthesis of 1-(4-methoxybenzyl)-3-ethyl-6-(4-methylpiperazin-1-yl)-1H-indazole

Into a 250 mL sealed tube was placed a solution of 1-(4-methoxybenzyl)-6-bromo-3-ethyl-1H-indazole (4 g, 11.59 mmol) in toluene (50 mL). To this was added 1-methylpiperazine (3.48 g, 34.80 mmol). Addition of Pd(OAc)₂ (26 mg, 0.12 mmol) was next. This was followed by the addition of Cs₂CO₃ (9.45 g, 29.01 mmol). To the mixture was added BINAP (220 mg, 0.35 mmol) and the N₂ was bubbled at the same time. The resulting solution was allowed to react, with stirring, overnight while the temperature was maintained at 120° C. in a bath of oil. A filtration was performed. The filtrate was concentrated by evaporation under vacuum using a rotary evaporator. The residue was purified by eluting through a column with a 1:100˜1:20 CH3OH: CH₂Cl₂ solvent system. This resulted in 3.4 g (73%) of 1-(4-methoxybenzyl)-3-ethyl-6-(4-methylpiperazin-1-yl)-1H-indazole as brown oil.

### 2. Synthesis of ethyl-6-(4-methylpiperazin-1-yl)-1H-indazole

Into a 150 mL sealed tube was placed a solution of 1-(4-methoxybenzyl)-3-ethyl-6-(4-methylpiperazin-1-yl)-1H-indazole (3.4 g, 8.41 mmol) in CF₃COOH (600 mL). To the mixture was added H₂O (6 mL). The resulting solution was allowed to react, with stirring, overnight while the temperature was maintained at 105° C. in a bath of oil. The reaction progress was monitored by TLC (CH₂Cl₂/MeOH=10:1). The mixture was concentrated by evaporation under vacuum using a rotary evaporator. Adjustment of the pH to 9 was accomplished by the addition of NaOH solution. The resulting solution was extracted six times with 100 mL of CH₂Cl₂ and the organic layers combined and dried over MgSO₄. The residue was purified by eluting through a column with a 1:100˜1:10 CH₂Cl₂/MeOH solvent system. This resulted in 0.89 g (43.39%) of 3-ethyl-6-(4-methylpiperazin-1-yl)-1H-indazole as a red solid.

¹H NMR(300 MHz, CDCl₃) δ7.58(d, 1H), 6.88(m, 1H), 6.80(d, 1H), 3.34(m, 4H), 2.97(m, 2H), 2.72(s, 4H), 2.45(s, 3H), 1.40(m, 3H). m/z 245 [M+H]⁺

### General Procedure J

### Synthesis of ethyl 1-{[3-(3-methoxypyrrolidin-1-yl)phenyl]sulfonyl}-6-piperazin-1-yl-1H-indazole-3-carboxylate

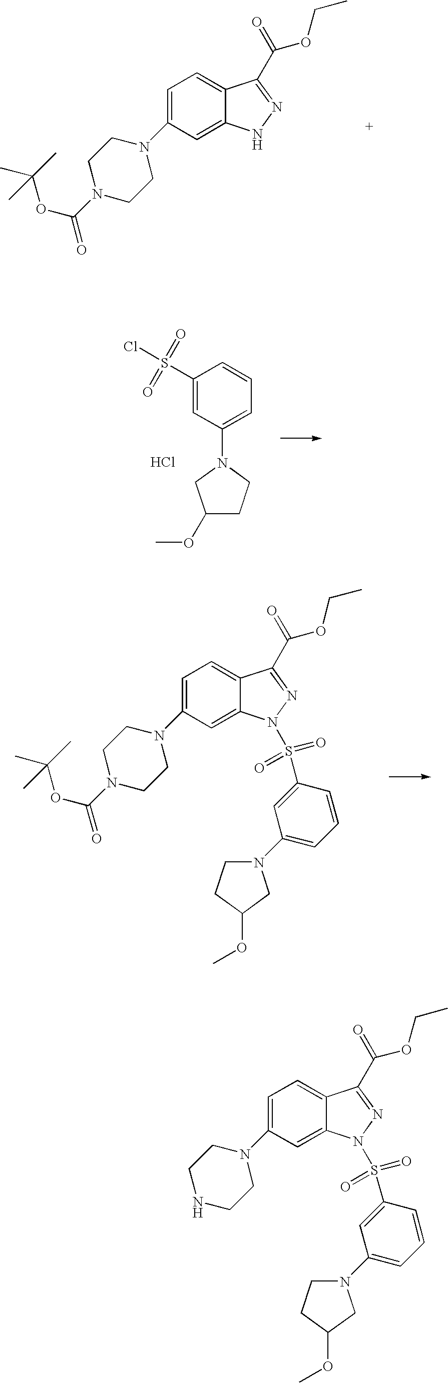

Into a vial was added ethyl 6-[4-(tert-butoxycarbonyl)piperazin-1-yl]-1H-indazole-3-carboxylate (100 mg, 0.0003 mol) and tetrahydrofuran (2 mL, 0.02 mmol) and N,N-Dimethylformamide (2 mL, 0.03 mol) at 5° C. and 1.0 M of sodium bis(trimethylsilyl)amide) in tetrahydrofuran (0.401 mL) was added and was stirred for 30 minutes. The solution was transferred into a Vial with 3-(3-methoxypyrrolidin-1-yl)benzenesulfonyl chloride (0.110 g, 0.000401 mol) and Tetrahydrofuran (2 mL, 0.02 mol) and N,N-dimethylethylamine (43.4 uL, 0.000401 mol) (DME is not needed for the neutral form of sulfonic chloride) at 5° C. and was stirred for 30 minutes. The reaction was extracted with ethyl acetate and was washed with water and brine and was rotovaped. The crude was adsorbed onto silica gel and was flash chromatographed on silica gel on a 12 g cartridge using a hexane:ethyl acetate gradient (10-60%) over minutes at a flow rate of 20 mL/min and UV detection at 254 nm. 126 mg was recovered. Ethyl 6-[4-(tert-butoxycarbonyl)piperazin-1-yl]-1-{[3-(3-methoxypyrrolidin-1-yl)phenyl]sulfonyl}-1H-indazole-3-carboxylate (0.12 g, 0.00020 mol) was added into a l-neck round-bottom flask. Additionally, one of the following steps was performed.

Acetonitrile (3 mL, 0.06 mol) and iodotrimethylsilane (56 uL, 0.00039 mol) were added. The reaction was stirred for 45 minutes at room temperature. The solvent was rotovaped at room temperature The reaction was diluted with water/acetonitrile (3.0 mL) and filtered through a 0.45 urn filter. The filtrate was purified on a C18 Sunfire column (30×100 mm) using a gradient of(10-80%) acetonitrile:water (with 0.1% formic acid) and a flow rate of 45 mL/min. Detection was performed by m/z=514. Fractions of interest were pooled and concentrated on a freeze drier. 45 mg as an amorphous yellow solid was recovered. LC-MS (1080-8min) M+1t=514.0 at 4.72 minutes, the reactants were treated with CF₃CO₂H and concentrated to form the corresponding CF₃CO₂H salt, or the reactants were treated with HCl in dioxane and concentrated to form the corresponding HCl salt.

¹H NMR (CD₃OD) δ 1.4 (t, 3H), 2.1 (m, 2H), 3.4-3.5 (m, 7H), 3.6 (m, 4H), 4.1 (s, 1H), 4.4 (q, 2H), 4.9 (s, 4H), 6.9 (d, 1H), 7.1 (s, 1H), 7.2 (d, 1H), 7.3 (m, 1H), 7.6 (s, 1H), 8.0 d, 1H), 8.5(br s, 1H). (step (1)).

Using this general procedure, the following compounds were prepared using different starting materials: 1, 2, 3, 5, 6, 11, 13, 33, 34, 35, 36, 40, 41, 42, 43, 44, 45, 50, 54, 55, 59, 60, 61, 62, 97, 100, 102, 104, 107, 112, and 114-141.

### General Procedure K

### Synthesis of 1-[(1-actyl-2,3-dihydro-1H-indol-5-yl)sulfonyl]-6-(4-methylpiperazin-1-yl)-1H-indazole

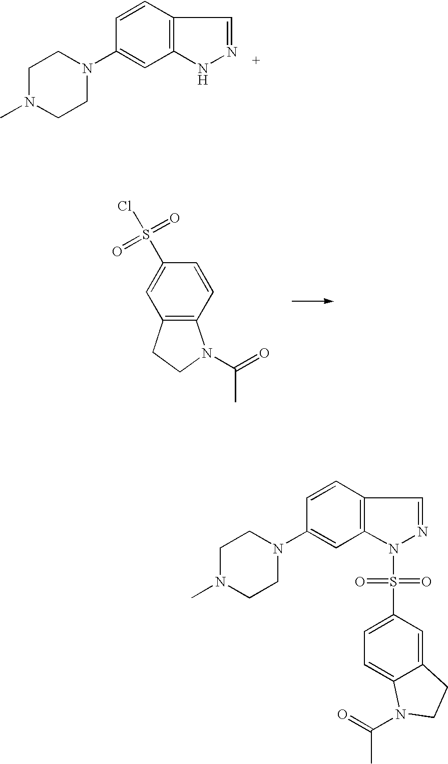

Into a vial was added 6-(4-methylpiperazin-1-yl)-1H-indazole (50.5 mg, 0.000233 mol) and N,N-Dimethylformamide (1 mL, 0.01 mol). The reaction was stirred at 5° C. and 1.0 M of sodium bis(trimethylsilyl)amide in tetrahydrofuran (3.50 mL) was added and was stirred for 30 minutes. 1-acetylindoline-5-sulfonyl chloride (90.9 mg, 0.000350 mol), (1.0 equiv. of N,N-dimethylethylamine was also added for the reaction involving the sulfonic chloride with HCl salt) was added and was stirred for 30 minutes. LC-MS (20-80% acetonitrile/water, Smin) shows product. The reaction was diluted with water/acetonitrile (1.0 mL) and filtered through a 0.45 um filter. The filtrate was purified on a C18 Sunfire column (30×100 mm) using a gradient of (20-80%) acetonitrile:water (with 0.1% formic acid) and a flow rate of 45 mL/min. Detection was performed by m/z=440.3. Fractions of interest were pooled and concentrated on a Genevac.

¹H NMR (CD3OD) δ 2.2 (m, 5H), 2.2 (m, 4H), 3.2 (m, 2H), 3.6 (s, 4H), 4.2 (m, 3H), 7.1 (d, 1H), 7.5-7.7 (m, 3H), 7.8 (m, 1H), 8.1-8.2 (m, 3H).

Using this general procedure, the following compounds were prepared using different starting materials: compounds 4, 7-10, 12, 14-32, 37-39, 46-49, 51-53, 56-58, 63, 64, 96, 98, 99, 101, 103, 104, 106, 108, and 113.

### Synthesis of Sulfonyl Chlorides

### Example 1

### Synthesis of 2-methyl-1,2,3,4-tetrahydroisoquinoline-8-sulfonyl chloride

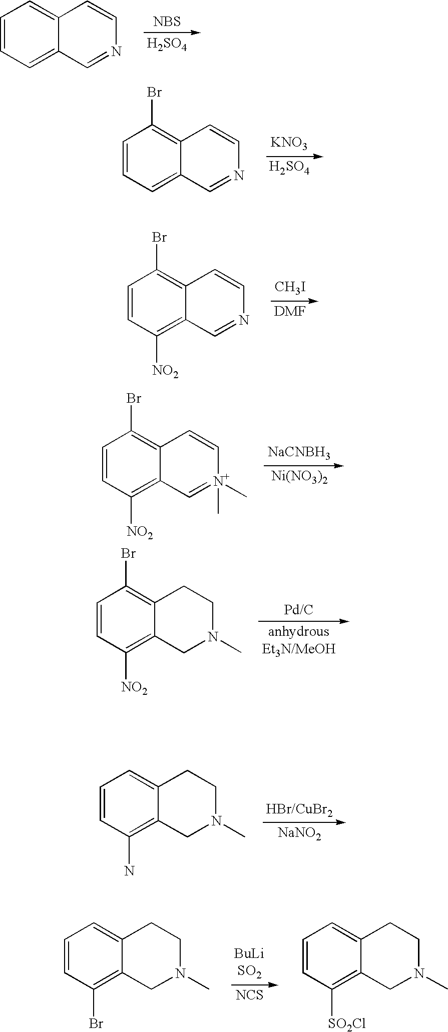

### Synthesis of 5-bromoisoquinoline

Into a 250 mL 3-necked round-bottom flask was placed H₂SO₄ (150 mL). To the above was added isoquinoline (17 g, 131.62 mmol) in several batches, while cooling to a temperature of 0° C. To the above was added NBS (29.2 g, 164.04 mmol) in several batches, while cooling to a temperature of −25-22° C. The resulting solution was allowed to react, with stirring, for 2 hours while the temperature was maintained at −25-22° C. The resulting solution was allowed to react, with stirring, overnight while the temperature was maintained at room temperature. The reaction progress was monitored by TLC (ethyl acetate/petroleum ether=1:5). The reaction mixture was then quenched by the adding 1000 mL of H₂O/ice. Adjustment of the pH to 8-10 was accomplished by the addition of NH₃. H₂O (30%). The resulting solution was extracted four times with 500 mL of ethyl acetate and the organic layers combined and dried over Na₂SO₄. The residue was purified by eluting through a column with a 1:5 ethyl acetate/petroleum ether solvent system. This resulted in 22.24 g (81%) of 5-bromoisoquinoline as a white solid.

### Synthesis of 5-bromo-8-nitroisoquinoline

Into a 500 mL 3-necked round-bottom flask was placed a solution of 5-bromoisoquinoline (22.24 g, 106.87 mmol) in H₂SO₄ (120 mL). This was followed by the addition of a solution of KNO3 (15.1 g, 149.36 mmol) in H₂SO₄ (100 mL), which was added dropwise with stirring, while cooling to a temperature of 20° C. over a time period of 1 hour. The resulting solution was allowed to react, with stirring, for 1 hour while the temperature was maintained at room temperature. The reaction progress was monitored by TLC (ethyl acetate/petroleum ether=1:5). The reaction mixture was then quenched by the adding 600 mL of H₂O/ice. Adjustment of the pH to 8-10 was accomplished by the addition of NH₃. H₂O (30%). A filtration was performed. The filter cake was washed 2 times with 500 mL of H₂O. The solid was dried in an oven under reduced pressure. This resulted in 25.59 g (90%) of 5-bromo-8-nitroisoquinoline as a yellow solid.

### Synthesis of 5-bromo-8-nitro-N-methylisoquinolinium iodide

Into a 500 mL round-bottom flask was placed a solution of 5-bromo-8-nitroisoquinoline (25.59 g, 101.11 mmol) in DMF (200 mL). To the mixture was added iodomethane (71.8 g, 505.99 mmol). The resulting solution was allowed to react, with stirring, overnight while the temperature was maintained at 40° C. A filtration was performed. The filter cake was washed 2 times with 250 mL of ethyl ether. This resulted in 33.33 g (83%) of 5-bromo-8-nitro-N-methylisoquinolinium iodide as a red solid.

### Synthesis of 5-bromo-2-methyl-8-nitro-1,2,3,4-tetrahydroisoquinoline

Into a 500 mL 3-necked round-bottom flask was placed a solution of Ni(NO3)2.6 H₂O (12.6 g, 43.33 mmol) in CH₃OH (200 mL). To the mixture was added 5-bromo-8-nitro-N-methylisoquinolinium iodide (33.33 g, 84.38 mmol). To the above was added NaCNBH3 (10.6 g, 168.68 mmol) in several batches. The resulting solution was allowed to react, with stirring, for 5 hours while the temperature was maintained at room temperature. The reaction progress was monitored by TLC (ethyl acetate/petroleum ether=1:5). The resulting solution was concentrated by evaporation under vacuum using a rotary evaporator. The residue was dissolved with 800 mL of H₂O. Adjustment of the pH to 8-10 was accomplished by the addition of NaOH (5%). A filtration was performed. The resulting solution was extracted 2 times with 800 mL of ethyl acetate and the organic layers combined and dried over Na₂SO₄. The residue was purified by eluting through a column with a 1:5 ethyl acetate/petroleum ether solvent system. This resulted in 19.3 g (83%) of 5-bromo-2-methyl-8-nitro-1,2,3,4-tetrahydroisoquinoline as a yellow solid.

### Synthesis of 2-methyl-1,2,3,4-tetrahydroisoquinolin-8-amine

Into a 250 mL 3-necked round-bottom flask was added a solution of 5-bromo-2-methyl-8-nitro-1,2,3,4-tetrahydroisoquinoline (4.85 g, 17.89 mmol) in CH30HEt₃N(anhydrous) (150/15 mL). To the mixture was added Pd/C((4.5 g) followed by hydrogen. The resulting solution was allowed to react, with stirring, for 3 hours while the temperature was maintained at room temperature. The reaction progress was monitored by TLC (ethyl acetate/petroleum ether=1:1). A filtration was performed. The filtrate was concentrated by evaporation under vacuum using a rotary evaporator. The resulting solution was diluted with 50 mL of Na₂CO₃ (10%). The resulting solution was extracted four times with 50 mL of Ethyl acetate and the organic layers combined and dried over Na₂SO₄. The residue was purified by eluting through a column with a 50:1 CH₂Cl₂/MeOH solvent system. This resulted in 2.57 g (89%) of 2-methyl-1,2,3,4-tetrahydroisoquinolin-8-amine as a light yellow oil.

### Synthesis of 8-bromo-2-methyl-1,2,3,4-tetrahydroisoquinoline

Into a 50 mL 3-necked round-bottom flask (named A), was placed 2-methyl-1,2,3,4-tetrahydroisoquinolin-8-amine (500 mg, 3.08 mmol). This was followed by the addition of a solution of HBr (5 mL) in H₂O (5 mL), which was added dropwise with stirring, while cooling to a temperature of 0° C. To the above was added NaNO₂ (230 mg, 3.33 mmol) in several batches, while cooling to a temperature of 0° C. and the mixture was stirred for 30mins at that temperature. Then into another 50 mL 3-necked round-bottom flask (named B), was purged and maintained with an inert atmosphere of nitrogen, was placed a solution of CuBr (550 mg, 3.83 mmol) in HBr/ H₂O (3mol/L) (10 mL), while cooling to a temperature of 0° C. The mixture was stirred for 10 minutes. Then was followed by the addition of the reaction solution of flask A, added dropwise while the temperature was maintained at 0° C. The resulting solution was allowed to react, with stirring, for 30 mins while the temperature was maintained at 0° C. The resulting solution was allowed to react, with stirring, for an additional 2 hours while the temperature was maintained at room temperature. The reaction progress was monitored by TLC(ethyl acetate/petroleum ether=1:1). Adjustment of the pH to 9 was accomplished by the addition of NaOH (10%). The resulting solution was extracted three times with 50 mL of CH₂Cl₂ and the organic layers combined and dried over K2CO3. A filtration was performed. The filtrate was concentrated by evaporation under vacuum using a rotary evaporator. The residue was purified by eluting through a column with a 1:1 ethyl acetate/petroleum ether solvent system. This resulted in 0.45 g (65%) of 8-bromo-2-methyl-1,2,3,4-tetrahydroisoquinoline as light yellow oil.

### Synthesis of 2-methyl-1,2,3,4-tetrahydroisoquinoline-8-sulfonyl chloride

Into a 100 mL 3-necked round-bottom flask purged and maintained with an inert atmosphere of nitrogen, was placed a solution of 8-bromo-2-methyl-1,2,3,4-tetrahydroisoquinoline (3 g, 13.27 mmol) in THF (30 mL). To the above was added 2.5M n-BuLi/hexane(6.9 mL), while cooling to a temperature of −78° C. over a time period of 15 minutes. The resulting solution was allowed to react, with stirring, for 40 minutes while the temperature was maintained at −78° C. Addition of SO2 (890 mg, 13.91 mmol) was next, while cooling to a temperature of −100° C. The resulting solution was allowed to react, with stirring, for 20 minutes while the temperature was maintained at −78° C. The resulting solution was allowed to react, with stirring, for an additional 1 hour while the temperature was maintained at room temperature. This was followed by the addition of n-hexane (60 mL). Then a filtration was performed. A light yellow solid was obtained. In another 250 mL 3-necked round-bottom flask was placed the above filter cake and CH₂Cl₂ (80 mL). To the above was added NCS (2.7 g, 20.22 mmol) in several batches, while cooling to a temperature of −10-0° C. The resulting solution was allowed to react, with stirring, for an additional 1 hour while the temperature was maintained at room temperature. The reaction progress was monitored by TLC(ethyl acetate/petroleum ether=3:2). The resulting mixture was washed 2 times with 100 mL of saturated NaHSO3 and 2 times with 50 mL of saturated NaCl. The mixture was dried over Na₂SO₄. A filtration was performed. The filtrate was concentrated by evaporation under vacuum using a rotary evaporator. This resulted in 1.44 g (44%) of 2-methyl-1,2,3,4-tetrahydroisoquinoline-8-sulfonyl chloride as a light yellow solid.

¹H NMR (300 MHz, DMSO) δ 7.63 (1H,d), 7.22 (2H,m), 5.03 (1H,d), 4.4(1H,m), 3.6 (1H,d), 3.34 (1H,d), 2.94 (2H,m), 2.49 (3H,s). m/z 246 [M+1]⁺

### Example 2

### Synthesis of 4-methyl-3,4-dihydro-2H-benzo[b][1,4]oxazine-6-sulfonyl chloride

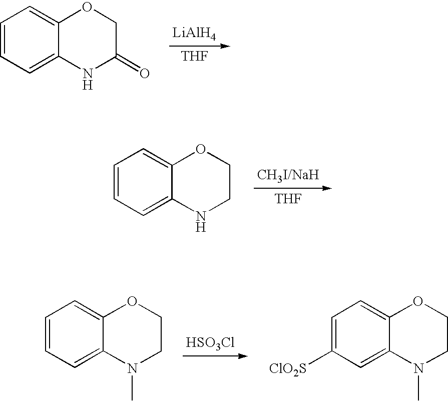

### Synthesis of 3,4-dihydro-2H-benzo[b][1,4]oxazine

Into a 250 mL 3-necked round-bottom flask, was placed a solution of lithium aluminum hydride (3.6 g, 94.74 mmol) in THE (80 mL). The mixture was stirred for 15 minutes. This was followed by the addition of a solution of 2H-benzo[b][1,4]oxazin-3(4H)-one (5.7 g, 38.22 mmol) in THF (21 mL), which was added dropwise with stirring. The resulting solution was allowed to react, with stirring, overnight while the temperature was maintained at reflux in a bath of oil. The reaction progress was monitored by TLC (ethyl acetate/petroleum ether=1:1). The reaction mixture was then quenched by the adding 3.6 mL of H₂O and 10.8 mL 15%NaOH. A filtration was performed. The filter cake was washed 1 time with 30 mL of THF. The resulting solution was extracted two times with 100 mL of ethyl acetate and the organic layers combined and dried over Na₂SO₄ and concentrated by evaporation under vacuum using a rotary evaporator. This resulted in 4.8 g (79%) of 3,4-dihydro-2H-benzo[b][1,4]oxazine as red oil.

### 2. Synthesis of 4-methyl-3,4-dihydro-2H-benzo b][1,4]oxazine

Into a 250 mL 3-necked round-bottom flask was placed a solution of 3,4-dihydro-2H-benzo[b[1,4]oxazine (4.8 g, 35.51 mmol) in THF (50 mL). To the above was added NaH (2.3 g, 57.50 mmol) in several batches, while cooling to a temperature of 0-5° C. The mixture was stirred for 30 minutes at 0-5° C. To the above was added iodomethane (9.0 g, 63.41 mmol) dropwise with stirring, while cooling to a temperature of 0-5° C. The resulting solution was allowed to react, with stirring, overnight while the temperature was maintained at room temperature. The reaction progress was monitored by TLC (ethyl acetate/petroleum ether=1:2). A filtration was performed. The filtrate was concentrated by evaporation under vacuum using a rotary evaporator. The residue was purified by eluting through a column with a 1:100 ethyl acetate/petroleum ether solvent system. This resulted in 3.0 g (50%)of 4-metliyl-3,4-dihydro-2H-benzo[b][1,4]oxazine as yellow oil.

### 3. Synthesis of 4-methyl-3,4-dihydro-2H-benzo[b][1,4]oxazine-6-sulfonyl chloride

Into a 250 mL 3-necked round-bottom flask was placed HSO3Cl (25 mL). To the above was added 4-methyl-3,4-dihydro-2H-benzo[b][1,4]oxazine (5.8 g, 38.93 mmol) dropwise with stirring, cooling to a temperature of 0-5° C. The resulting solution was allowed to react, with stirring, for 120 minutes while the temperature was maintained at room temperature. The reaction progress was monitored by TLC (ethyl acetate/petroleum ether=1:2). The reaction mixture was then quenched by the adding of H₂O/ice. The resulting solution was extracted three times with 200 mL of ethyl acetate and the organic layers combined and dried over Na₂SO₄ and concentrated by evaporation under vacuum using a rotary evaporator. The resulting mixture was washed 3 times with 15 mL of hexane. This resulted in 2.9 g (27%) of 4-methyl-3,4-dihydro-2H-benzo[b][1,4]oxazine-6-sulfonyl chloride as a light yellow solid.

¹H NMR (300 MHz, CDCl₃)δ 2.98(s, 3H), 3.36(m, 2H), 4.38(m, 2H), 6.87(d, 1H), 7.19(s, 1H), 7.34(d, 1H). m/z 319 [M+BnNH+H]⁺

### Example 3

### Synthesis of 2-oxo-1,2,3,4-tetrahydroquinoline-7-sulfonyl chloride

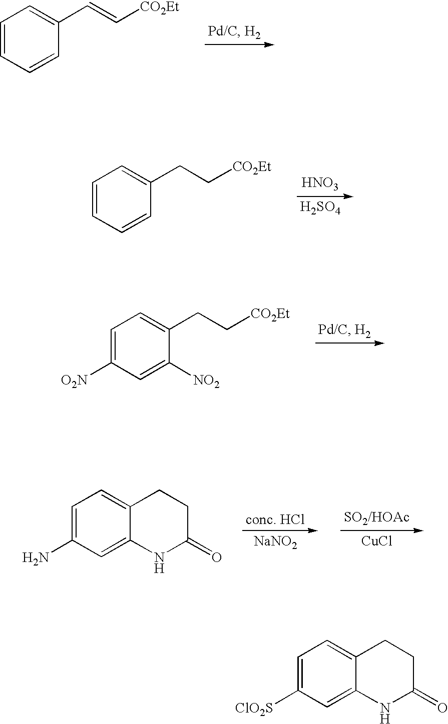

### Synthesis of ethyl 3-phenylpropanoate

Into a 500 mL 3-necked round-bottom flask was added a solution of ethyl cinnamate (10 g, 56.75 mmol) in MeOH (200 mL). To the mixture was added Pd/C (2 g) followed by hydrogen. The resulting solution was allowed to react, with stirring, overnight while the temperature was maintained at 35° C. in a bath of oil. A filtration was performed. The filtrate was concentrated by evaporation under vacuum using a rotary evaporator. This resulted in 10 g (99%)of ethyl 3-phenylpropanoate as a colorless oil.

### 2. Synthesis of ethyl 3-(2,4-dinitrophenyl)propanoate

Into a 250 mL 3-necked round-bottom flask, was placed a solution of fuming HNO3 (25 mL) in cone. H₂SO₄ (50 mL). To the mixture was added ethyl 3-phenylpropanoate (5 g, 28.09 mmol), while cooling to a temperature of 0° C. The resulting solution was allowed to react, with stirring, for 1 hour while the temperature was maintained at 0° C. The resulting solution was allowed to react, with stirring, overnight while the temperature was maintained at 60° C. The reaction progress was monitored by TLC (ethyl acetate/petroleum ether=1:3). The reaction mixture was then quenched by the adding of H₂O/ice. The resulting solution was extracted two times with 50 mL of ethyl acetate and the organic layers combined. The resulting mixture was washed 2 times with 50 mL of NaHCO3(aq.). The mixture was dried over MgSO₄ and concentrated by evaporation under vacuum using a rotary evaporator. This resulted in 2 g (27%) of ethyl 3-(2,4-dinitrophenyl)propanoate as a yellow solid.

### 3. Synthesis of 7-amino-3,4-dihydroquinolin-2(1H)-one

Into a 100 mL 3-necked round-bottom flask, was placed a solution of ethyl 3-(2,4-dinitrophenyl)propanoate (1.5 g, 5.60 mmol) in MeOH (20 mL). To the mixture was added Pd/C (0.5 g). Hydrogen gas was passed through. The resulting solution was allowed to react, with stirring, overnight while the temperature was maintained at 30° C. A filtration was performed. The filtrate was concentrated by evaporation under vacuum using a rotary evaporator. This resulted in 0.5 g (55%) of 7-amino-3,4-dihydroquinolin-2(1H)-one as a green-yellow solid.

### 4. Synthesis of 2-oxo-1,2,3,4-tetrahydroquinoline-7-sulfonyl chloride

Into a 50 mL 3-necked round-bottom flask was placed a solution of 7-amino-3,4-dihydroquinolin-2(1H)-one (350 mg, 2.16 mmol) in concentrated HCl (6 mL). This was followed by the addition of a solution of sodium nitrite (200 mg, 2.90 mmol) in H₂O (2 mL) at −5-0° C. The mixture was stirred for 30 min. Then the resulting solution was added into a solution of copper chloride (200 mg, 2.02 mmol) in CH₃COOH (10 mL) that was saturated with SO2 gas. The resulting solution was allowed to react, with stirring, for 1 hour while the temperature was maintained at 10-30° C. The reaction progress was monitored by TLC (CH₂Cl2/MeOH=10:1). The reaction mixture was then quenched by the adding of H₂O/ice. The resulting solution was extracted two times with 20 mL of Ethyl acetate and the organic layers combined. The resulting mixture was washed 2 times with 10 mL of H₂O and 1 time with 10 mL of NaHCO₃/H₂O. The mixture was dried over Na₂SO₄. A filtration was performed. The filtrate was concentrated by evaporation under vacuum using a rotary evaporator. This resulted in 0.24 g (45%) of 2-oxo-1,2,3,4-tetrahydroquinoline-7-sulfonyl chloride as a brown solid.

¹H NMR (300 MHz, CDCl3) δ 2.89(m, 2H), 2.95(m, 2H), 7.41(m, 1H),7.43(m, 1H), 7.47(m, 1H). m/z 315 [M−H]⁻

### Example 4

### Synthesis of 3-(3-methoxypyrrolidin-1-yl)benzene-1-sulfonyl chloride

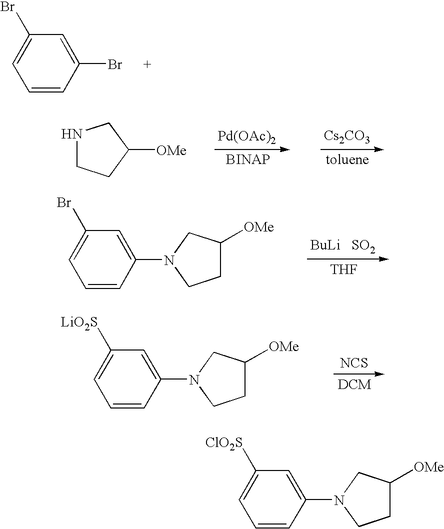

### Synthesis of 1-(3-bromophenyl)-3-methoxypyrrolidine

Into a 250 mL 3-necked round-bottom flask purged and maintained with an inert atmosphere of nitrogen, was placed a solution of 1,3-dibromobenzene (11.9 g, 50.42 mmol) in toluene (100 mL). To this was added 3-methoxypyrrolidine (6.1 g, 60.40 mmol). Addition of Pd(OAc)2 (113 mg, 0.50 mmol) was next. This was followed by the addition of BINAP (940 mg, 1.51 mmol). To the mixture was added Cs₂CO₃ (40.9 g, 125.54 mmol). The resulting solution was allowed to react, with stirring, overnight while the temperature was maintained at reflux in a bath of oil. The reaction progress was monitored by TLC (Ethyl acetate/PE=1:5). A filtration was performed. The filtrate was concentrated by evaporation under vacuum using a rotary evaporator. The residue was purified by eluting through a column with a 1:30 ethyl acetate/petroleum ether solvent system. This resulted in 8.3 g (64.3%) of 1-(3-bromophenyl)-3-methoxypyrrolidine as yellow oil.

### 2. Synthesis of lithium 3-(3-methoxypyrrolidin-1-yl)benzenesulfinate

Into a 250 mL 3-necked round-bottom flask purged and maintained with an inert atmosphere of nitrogen, was placed a solution of 1-(3-bromophenyl)-3-methoxypyrrolidine (8.3 g, 32.42 mmol) in TM (100 RL). To this was added BuLi (15.6 mL).The resulting solution was allowed to react with stirring, for 1 hour while the temperature was maintained at −78° C. in a bath of N2( liquid ). (Walter—shouldn't this be dry ice/acetone instead of liquid nitrogen?) To the mixture was added SO2 (4 mL). The resulting solution was allowed to react, with stirring, for an additional 2 hours while the temperature was maintained at −78° C. in a bath of N2( liquid ).(Walter—same as before) The reaction progress was monitored by TLC (ethyl acetate/petroleum ether)=1:1). The mixture was concentrated by evaporation under vacuum using a rotary evaporator. The product was precipitated by the addition of Hexane. A filtration was performed. The filter cake was washed 2 times with 50 mL of hexane. The solid was dried in an oven under reduced pressure. This resulted in 12 g (90%)of lithium 3-(3-methoxypyrrolidin-1-yl)benzenesulfinate as a yellow solid.

### 3. Synthesis of 3-(3-methoxypyrrolidin-1-yl)benzene-1-sulfonyl chloride

Into a 250 mL round-bottom flask, was placed a solution of lithium 3-(3-methoxypyrrolidin-1-yl)benzenesulfinate (12 g, 29.15 mmol) in CH₂Cl₂ (100 mL). To the above was added NCS (4.48 g, 33.56 mmol) in several batches, while cooling to a temperature of 0° C. over a time period of 10 minutes. The resulting solution was allowed to react, with stirring, for 15 minutes while the temperature was maintained at 0° C. in a bath of H₂O/ice, then the ice bath was removed and the solution was allowed to react, for an additional 25 minutes while the temperature was maintained at room temperature. The reaction progress was monitored by TLC (ethyl acetate/petroleum ether=1:1). The resulting mixture was washed 2 times with 50 mL of NaHSO3 and 2 times with 50 mL of brine. The mixture was dried over Na₂SO₄ and concentrated by evaporation under vacuum using a rotary evaporator. The residue was purified by eluting through a column with a 2:3 ethyl acetate/petroleum ether solvent system. This resulted in 6.6 g (82.5%) of 3-(3-methoxypyrrolidin-1-yl)benzene-1-sulfonyl chloride as yellow oil.

¹H NMR(400 Hz, CDCl3) δ 2.24(1H,m), 2.30(m, 1H) 3.54-3.45(m, 2H) 3.61-3.56(m, 2H), 4.2(s, 3H), 6.90(d, 1H, J=8 Hz), 7.34(s, 1H, J=8 Hz), 7.36(dd, 1H, J=8 Hz), 7.48(dd, 1H, J=8,8 Hz). m/z 347 [M+BnNH+H]⁺

### Example 5

### Synthesis of 3-oxo-3,4-dihydro-2H-benzo[b][1,4]oxazine-6-sulfonyl chloride

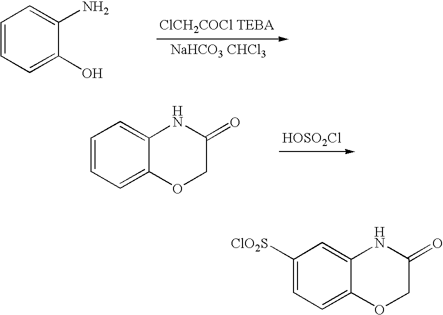

### Synthesis of 2H-benzo[b][1,4]oxazin-3(4H)-one

Into a 100 mL round-bottom flask was placed a solution of 2-aminophenol (5.45 g, 49.98 mmol) in CHCl3 (30 mL). To this was added TEBA (Walter—abbreviation TEBA?) (11.4 g, 50.00 mmol). To the mixture was added NaHCO3 (16.8 g, 200.00 mmol). This was followed by the addition of a solution of 2-chloroacetyl chloride (8.16 g, 72.21 mmol) in CHCl3 (5 mL), which was added dropwise with stirring, while cooling to a temperature of 0° C. over a time period of 20 minutes. The resulting solution was allowed to react, with stirring, for 1 hour while the temperature was maintained at 0-5° C. The resulting solution was allowed to react, with stirring, overnight while the temperature was maintained at 55° C. The mixture was concentrated by evaporation under vacuum using a rotary evaporator. The product was precipitated by the addition of H₂O. A filtration was performed. The filter cake was washed 2 times with 50 mL of H₂O. The final product was purified by recrystallization from EtOH. This resulted in 4.5 g (60%) of 2H-benzo[b][1,4]oxazin-3(4H)-one as a white solid.

### 2. Synthesis of 3-oxo-3,4-dihydro-2H-benzo[b][1,4]oxazine-6-sulfonyl chloride

Into a 100 mL round-bottom flask was placed HSO3Cl (10 mL). To the above was added 2H-benzo[b][1,4]oxazin-3(4H)-one (2 g, 13.42 mmol) in several batches, while cooling to a temperature of 0-5° C. over a time period of 20 minutes. The resulting solution was allowed to react, with stirring, for 1 hour while the temperature was maintained at 5-10° C. The reaction mixture was poured into 100 g of ice carefully. The resulting solution was extracted one time with 100 mL of CH₂Cl₂ and the organic layers combined and dried over Na₂SO₄. A filtration was performed. The filtrate was concentrated by evaporation under vacuum using a rotary evaporator. This resulted in 2.2 g (66%) of 3-oxo-3,4-dihydro-2H-benzo[b][1,4]oxazine-6-sulfonyl chloride as a white solid.

¹H NMR (400 MHz, CDCl3) δ 9.29 (s, 1H), 7.71 (d, 2H), 7.52 (s, 1H), 7.16 (d, 2H), 4.80 (s, 2H). m/z 317 [M+BnNH−H]⁻

### Example 6

### Synthesis of 3-(3-(tetrahydro-2H-pyran-2-yloxy)pyrrolidin-1-yl)benzene-1-sulfonyl chloride

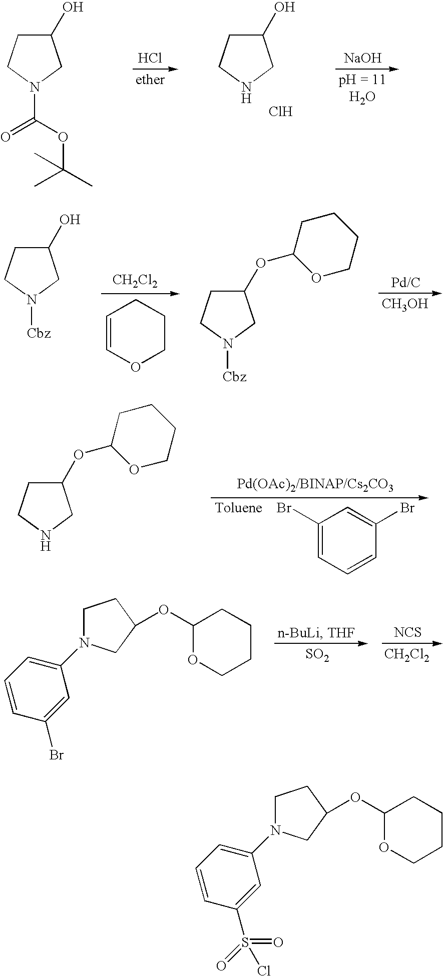

### Synthesis of pyrrolidin-3-ol hydrochloride

Into a 500 mL 3-necked round-bottom flask was placed a solution of tert-butyl 3-hydroxypyrrolidine-1-carboxylate (41 g, 218.97 mmol) in ethyl ether (300 mL). To the above was bubbled HCl (g), while maintaining at room temperature over a time period of 3 hours. The resulting solution was allowed to react, with stirring, overnight while the temperature was maintained at room temperature. The mixture was concentrated by evaporation under vacuum using a rotary evaporator. This resulted in 27 g (crude) of pyrrolidin-3-ol hydrochloride as a white solid.

### 2. Synthesis of benzyl 3-hydroxypyrrolidine-1-carboxylate

Into a 500 mL 3-necked round-bottom flask was placed a solution of pyrrolidin-3-ol hydrochloride (20.2 g, 163.43 mmol) in H₂O (60 mL) while cooling to 5° C. Adjustment of the pH to 7 was accomplished by the NaOH(10%). This was followed by the addition of a solution of benzyl chloroformate (36.8 g, 216.47 mmol), which was added dropwise with stirring, while cooling to a temperature of 5° C. The resulting solution was allowed to react, with stirring, for 2 hours at 5° C. Then the resulting solution was allowed to react, with stirring, for 1 hour while the temperature was maintained at room temperature. The reaction progress was monitored by TLC (ethyl acetate/petroleum ether=1:2). The resulting solution was extracted three times with 100 mL of ethyl acetate and the organic layers combined and dried over MgSO₄ and concentrated by evaporation under vacuum using a rotary evaporator. This resulted in 30 g (crude) of benzyl 3-hydroxypyrrolidine-1-carboxylate as brown oil.

### 3. Synthesis of benzyl 3-(tetrahydro-2H-pyran-2-yloxy)pyrrolidine-1-carboxylate

Into a 250 mL 3-necked round-bottom flask was placed a solution of benzyl 3-hydroxypyrrolidine-1-carboxylate (10 g, 45.23 mmol) in CH₂Cl₂ (100 mL). To this was added 3,4-dihydro-2H-pyran (19 g, 226.19 mmol). To the mixture was added P-TSA (389 mg, 2.26 mmol) and the resulting solution was allowed to react, with stirring, for 10 minutes while the temperature was maintained at 0° C. The resulting solution was allowed to react, with stirring, for an additional 1 hour at room temperature. The reaction progress was monitored by TLC (ethyl acetate/petroleum ether=1:2). The reaction mixture was then quenched by the adding 100 mL of NaHCO3. The resulting mixture was washed 1 time with 100 mL of NaHCO3 and I time with 100 mL of brine. The mixture was dried over MgSO₄ and concentrated under vacuum using a rotary evaporator. This resulted in 15 g (98%) of benzyl 3-(tetrahydro-2H-pyran-2-yloxy)pyrrolidine-1-carboxylate as yellow oil.

### 4. Synthesis of 3-(tetrahydro-2H-pyran-2-yloxy)pyrrolidine

Into a 250 mL round-bottom flask was placed a solution of benzyl 3-(tetrahydro-2H-pyran-2-yloxy)pyrrolidine-1-carboxylate (15 g, 44.26 mmol) and Pd/C (2.3 g) in CH3OH (absolute) (100 mL). Hydrogen gas was bubbled. The resulting solution was allowed to react, with stirring, for 2 hours while the temperature was maintained at room temperature. A filtration was performed. The filtrate was concentrated by evaporation under vacuum using a rotary evaporator. This resulted in 5.6 g (67%) of 3-(tetrahydro-2H-pyran-2-yloxy)pyrrolidine as a yellow liquid.

### 5. Synthesis of 1-(3-bromophenyl)-3-(tetrahydro-2H-pyran-2-yloxy)pyrrolidine

Into a 250 mL 3-necked round-bottom flask purged and maintained with an inert atmosphere of nitrogen, was placed a solution of 1,3-dibromobenzene (7.0 g, 29.91 mmol) in toluene (100 mL). To this was added 3-(tetrahydro-2H-pyran-2-yloxy)pyrrolidine (5.6 g, 32.75 mmol). Addition of Pd(OAc)2 (66.9 mg, 0.30 mmol) was next. This was followed by the addition of Cs₂CO₃ (24.27 g, 74.49 mmol). To the mixture was added BINAP (556 mg, 0.89 mmol). The resulting solution was allowed to react, with stirring, overnight while the temperature was maintained at reflux in a bath of oil. The reaction progress was monitored by TLC (ethyl acetate/petroleum ether=1:5). A filtration was performed. The filter cake was washed 3 times with 100 mL of brine. The mixture was dried over MgSO₄. The residue was purified by eluting through a column with a 1:100 ethyl acetate/petroleum ether solvent system. This resulted in 1.36 g (13%) of 1-(3-bromophenyl)-3-(tetrahydro-2H-pyran-2-yloxy)pyrrolidine as a yellow liquid.

### 6. Synthesis of 3-(3-(tetrahydro-2H-pyran-2-yloxy)pyrrolidin-1-yl)benzene-1-sulfonyl chloride

Into a 100 mL 3-necked round-bottom flask purged and maintained with an inert atmosphere of nitrogen, was placed a solution of 1-(3-bromophenyl)-3-(tetrahydro-2H-pyran-2-yloxy)pyrrolidine (1.4 g, 0.00429 mol) in THF (50 mL). To the above was added n-BuLi (2.16 mL) dropwise with stirring, while cooling to a temperature of −78° C. The resulting solution was allowed to react, with stirring, for 40 minutes at −78 degree C. To the mixture was added SO2 (450 mg, 0.00703 mol). The resulting solution was allowed to react, with stirring, for 60 minutes at −78˜40 degree C. Then 50 mL of n-hexane was added, and the solid was collected by filtration. Then the solid was suspended in 50 mL of CH₂Cl₂. To the above was added NCS (930 mg, 0.00697mol) in several batches, while cooling to a temperature of 0° C. The resulting solution was allowed to react, with stirring, for 40 minutes while the temperature was maintained at room temperature. The resulting mixture was washed 3 times with 100 mL of NaHSO3(2M) and 1 time with 100 mL of brine. The mixture was dried over MgSO₄. A filtration was performed. The filtrate was concentrated by evaporation under vacuum using a rotary evaporator This resulted in 1.0 g (61%) of 3-(3-(tetrahydro-2H-pyran-2-yloxy)pyrrolidin-1-yl)benzene-1-sulfonyl chloride as yellow oil.

¹H NMR(300 MHz, CDCl3) δ7.38(m,1H), 7.30(m, 1H), 7.10(s, 1H), 6.82(d, 1H),4.75(m, 1H), 4.52(m, 1H) ,3.90(m, 1H)3.38-3.57(m, 5H), 2.18(m, 1H), 2.05(m, 1H), 1.70-1.80(m, 2H), 1.55(d, 4H). m/z 417 [M+BnNH2+H]⁺.

### Example 7

### Synthesis of benzo[d]isoxazole-5-sulfonyl chloride

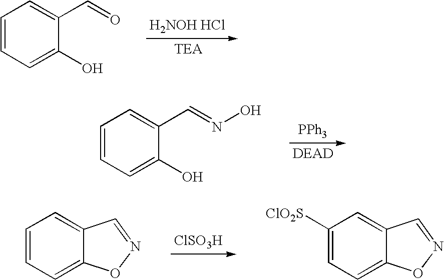

### Synthesis of (E)-2-hydroxybenzaldehyde oxime

Into a 500 mL round-bottom flask was placed a solution of 2-hydroxybenzaldehyde (20 g, 163.93 mmol) in ethanol (200 mL). To this was added hydroxylamine hydrochloride (14 g, 197.18 mmol). To the mixture was added triethylamine (19.2 g, 190.10 mmol) slowly. The resulting solution was allowed to react, with stirring, for 5 hours while the temperature was maintained at 95° C. in a bath of oil. The reaction progress was monitored by TLC (ethyl acetate/petroleum ether=1:2). The mixture was concentrated by evaporation. The resulting solution was extracted two times with 150 mL of ethyl acetate and water The resulting mixture was washed 3 times with 150 mL of water. The mixture was dried over MgSO₄ and concentrated by evaporator. The residue was purified by eluting through a column with a 1:100 ethyl acetate/petroleum ether solvent system. This resulted in 10 g (43%) of (E)-2-hydroxybenzaldehyde oxime as a white solid.

### 2. Synthesis of benzo[d]isoxazole

Into a 1 L 3-necked round-bottom flask, purged and maintained with an inert atmosphere of nitrogen, was placed a solution of (E)-2-hydroxybenzaldehyde oxime (3 g, 21.90 mmol) in Tri (300 mL). To the mixture was added PPh3 (6.024 g, 22.99 mmol), while cooling to a temperature of 4° C. This was followed by the addition of a solution of DEAD (4 g, 22.99 mmol) in THF (150 mL), while cooling to a temperature of 4° C. over a time period of 4 hours. The resulting solution was allowed to react with stirring, for 1 hour while the temperature was maintained at 4° C. in a bath of H₂O/ice. The reaction progress was monitored by TLC (ethyl acetate/petroleum ether=1:2). The mixture was concentrated by evaporation under vacuum using a rotary evaporator. The residue was purified by eluting through a column with a 1:100 ethyl acetate/petroleum ether solvent system. This resulted in 1.8 g (66%) of benzo[d]isoxazole as yellow oil.

### 3. Synthesis of benzo[d]isoxazole-5-sulfonyl chloride

Into a 50 mL round-bottom flask was placed ClSO₃H (2.8 mL). To the mixture was added benzo[d]isoxazole (500 mg, 4.20 ) dropwise at 0 degree C. The resulting solution was allowed to react, with stirring, for 27 hours while the temperature was maintained at 100° C. in a bath of oil. The reaction progress was monitored by TLC (ethyl acetate/petroleum ether=1:5). The reaction mixture was diluted by CH₂Cl₂ and poured into 50 mL of H₂O/ice cautiously. The aqueous layer was extracted two times with 50 mL of CH₂Cl₂ and the organic layers combined. The resulting mixture was washed 2 times with 50 mL of water. The mixture was dried over MgSO₄ and concentrated by evaporation under vacuum using a rotary evaporator. This resulted in 500 mg (48%) of benzo[d]isoxazole-5-suffonyl chloride as a red solid.

¹H NMR(300 MHz, CDCl3) δ 8.93(s, 1H), 8.54(s, 1H), 8.26(d, 1H), 7.87(d, 1H). m/z 287 [M+BnNH−]⁻

### Example 8

### Synthesis of isoquinoline-8-sulfonyl chloride

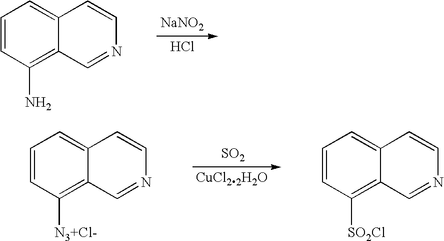

### Synthesis of isoquinoline-8-sulfonyl chloride

Into a 500 mL 4-necked round bottom flask was placed a solution of isoquinolin-8-amine (2.9 g, 16.09 mmol) in CH₃CN (100 mL). To this was added acetic acid (12 g, 199.67 mmol), while cooling to a temperature of −5-0° C. To the above was added HCl (6.1 g, 60.16 mmol) dropwise with stirring, while cooling to a temperature of −5-0° C. This was followed by the addition of a solution of NaNO₂ (1.67 g, 24.20 mmol) in H₂O (2 mL) and the mixture was stirred for 45 mins, while cooling to a temperature of −5-0° C. Then with SO2 gas was introduced for about 2 hours. This was followed by the addition of a solution of CuCl₂.2H₂O (3.6 g, 21.11 mmol) in H₂O (5 mL), while cooling to a temperature of −5-0° C. SO₂ was added to the mixture for about 1 hour. The resulting solution was allowed to react, with stirring, overnight while the temperature was maintained at 0-−5° C. in a bath of H₂O/ice. The reaction progress was monitored by TLC (ethyl acetate/petroleum ether=1:2). The reaction mixture was then quenched by the adding 400 mL of H₂O/ice. The resulting solution was extracted three times with 200 mL of CH₂Cl₂ and the organic layers combined and washed with brine and dried over Na₂SO₄ and concentrated by evaporation under vacuum using a rotary evaporator. The resulting mixture was washed 2 times with 10 mL of CH₂Cl₂. A filtration was performed. This resulted in 0.74 g (12%) of isoquinoline-8-sulfonyl chloride as a brown solid. m/z 228 [M+H]⁺

### Example 9

### Synthesis of 4-(2-oxopyrrolidin-1-yl)benzene-1-sulfonyl chloride

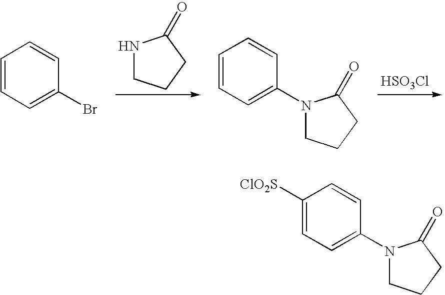

### Synthesis of 1-phenylpyrrolidin-2-one

Into a 150 mL sealed tube purged and maintained with an inert atmosphere of nitrogen, was placed 1-bromobenzene (4 g, 25.48 mmol). To this was added pyrrolidin-2-one (2.18 g, 25.65 mmol). Addition of Pd(OAc)2 (57 mg, 0.25 mmol) was next. This was followed by the addition of BINAP (240 mg, 0.39 mmol). This was followed by the addition of Cs₂CO₃ (12.5 g, 38.34 mmol). To the mixture was added toluene (50 mL). The resulting solution was allowed to react, with stirring, overnight while the temperature was maintained at 120° C. in a bath of oil. The mixture was concentrated by evaporation under vacuum using a rotary evaporator. The residue was purified by eluting through a column with a 1:10 ethyl acetate/petroleum ether solvent system. This resulted in 1 g (24%) of 1-phenylpyrrolidin-2-one as yellow oil.

### 2. Synthesis of 4-(2-oxopyrrolidin-1-yl)benzene-1-sulfonyl chloride

Into a 50 mL round-bottom flask was placed HSO3Cl (10 mL). To the mixture was added 1-phenylpyrrolidin-2-one (1 g, 6.21 mmol). The resulting solution was allowed to react, with stirring, overnight while the temperature was maintained at room temperature. The reaction mixture was then quenched by the adding 100 mL of H₂O/ice. The resulting solution was extracted one time with 100 mL of CH₂Cl₂ and the organic layers and dried over MgSO₄ and concentrated by evaporation under vacuum using a rotary evaporator This resulted in 0.7 g (43%) of 4-(2-oxopyrrolidin-1-yl)benzene-1-sulfonyl chloride as a yellow solid.

¹H NMR(400 MHz,CDCl3) δ 2.22(m, 2H), 2.71(t, 2H), 3.95(t, 2H), 7.88(t, 2H), 8.05(t, 2H). m/z 162 [M+H]⁺

### Example 10

### Synthesis of 3-oxo-3,4-dihydro-2H-benzo[b][1,4]oxazine-7-sulfonyl chloride

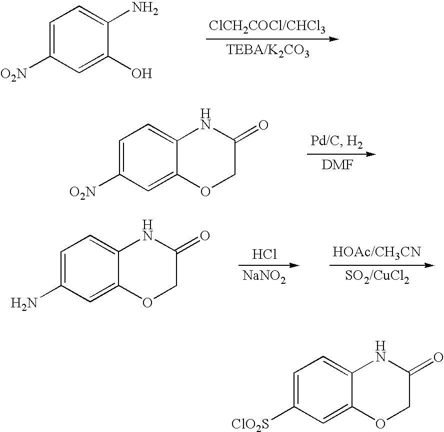

### Synthesis of 7-amino-2H-benzo[b][1,4]oxazin-3(4H)-one

Into a 500 mL 3-necked round-bottom flask was added a solution of 7-nitro-2H-benzo[b][1,4]oxazin-3(4H)-one (12 g, 61.86 mmol) in DMF (150 mL). To the mixture was added Pd/C (5 g) followed by hydrogen. The resulting solution was allowed to react, with stirring, overnight while the temperature was maintained at room temperature. The reaction progress was monitored by TLC (PE/EA=1:1). A filtration was performed. The filtrate was concentrated by evaporation under vacuum using a rotary evaporator. The product was precipitated by the addition of H₂O. A filtration was performed. The filter cake was washed 3 times with 300 mL of hexane. This resulted in 7.3 g (68%) of 7-amino-2H-benzo[b][1,4]oxazin-3(4H)-one as a yellow solid.

### 2. Synthesis of 3-oxo-3,4-dihydro-2H-benzo[b][1,4]oxazine-7-sulfonyl chloride

Into a 500 mL 3-necked round-bottom flask was placed a solution of 7-amino-2H-benzo[b][1,4]oxazin-3(4H)-one (5 g, 28.96 mmol) in CH₃CN (200 mL). To the above was added acetic acid (24.9 g) dropwise with stirring, while cooling to a temperature of 0° C. To the above was added HCl (16.2 g) dropwise with stirring, while cooling to a temperature of 0° C. This was followed by the addition of a solution of NaNO₂ (2.52 g, 36.52 mmol) in H₂O (2 mL), which was added dropwise with stirring, while cooling to a temperature of 0° C. The resulting solution was allowed to react, with stirring, for 30 minutes while the temperature was maintained at 0-−5° C. in a bath of H₂O/ice. This was followed by and maintained with an atmosphere of sulfur dioxide, the resulting solution was allowed to react, with stirring, for an additional 2 hours while the temperature was maintained at 0-−5° C. in a bath of H₂O/ice. To the mixture was added CuCl2.2 H₂O (5.11 g, 29.97 mmol), while cooling to a temperature of 0-5° C. The resulting solution was allowed to react, with stirring, maintained with an atmosphere of sulfur dioxide for an additional 2 hours while the temperature was maintained at 0-−5° C. in a bath of H₂O/ice. The resulting solution was allowed to react, with stirring, overnight while the temperature was maintained at room temperature. The reaction progress was monitored by TLC (ethyl acetate/petroleum ether=1:1). The reaction mixture was then quenched by the adding 200 mL of H₂O/ice. The resulting solution was extracted one time with 500 mL of ethyl acetate and the organic layers combined. Then the mixture was washed 3 times with 200 mL of brine. The mixture was dried over MgSO₄ and concentrated by evaporation under vacuum using a rotary evaporator. The residue was dissolved in 100 mL of CH₂Cl₂. A filtration was performed. The filtrate was concentrated by evaporation under vacuum using a rotary evaporator. This resulted in 0.9 g (11%) of 3-oxo-3,4-dihydro-2H-benzo[b][1,4]oxazine-7-sulfonyl chloride as a yellow solid.

¹H NMR (400 MHz, CDCl₃) δ: 4.73 (s, 2H), 7.00(m, 1H), 7.28(d, 1H), 7.71(d, 1H), 8.27(s, 1H).

### Example 11

### Synthesis of 3-(Dimethylamino)benzene-1-sulfonyl Chloride

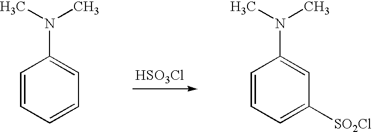

Sulfurochloridic acid (100 g, 862.07 mmol) was cooled to 0° C. and N,N-dimethylbenzenamine (20 g, 165.29 mmol) was added dropwise with stirring, maintaining a temperature of 0° C. The resulting solution was then heated to 120° C. and stirred for 3 h. After cooling to room temperature, dichloromethane (40 mL) was added and the resulting mixture was added dropwise to 100 mL of ice/salt water. The resulting solution was extracted with dichloromethane (3×500 mL) and the organic layers combined, dried (Na₂SO4) and filtered. The filtrate was concentrated and the residue was purified by column chromatography using a 1:100 ethyl acetate/petroleum ether solvent system. The collected fractions were combined and concentrated to give 4.1 g (11%) of 3-(dimethylamino)benzene-1-sulfonyl chloride as a yellow solid.

¹H NMR (CDCl₃, δ) 7.41 (t, 1H), 7.31 (d, 1H), 7.23 (s, 1H), 6.98 (m, 1H), 3.05 (s, 6H).

### Example 12

### Synthesis of 4-(Pyrrolidin-1-yl)benzene-1-sulfonyl Chloride

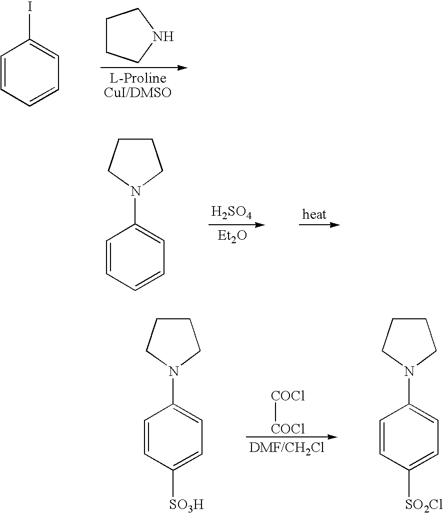

### Synthesis of 1-phenylpyrrolidine

Pyrrolidine (21.6 g, 304.23 mmol), L-proline (1.12 g, 9.74 mmol), and CuI (960 mg, 5.05 mmol) were added sequentially to 1-iodobenzene (10.0 g, 49.02 mmol). DMSO (40 mL) was then added, and the resulting solution was stirred at 60° C. for 20 h. The reaction mixture was then quenched by adding 400 mL of iced water. The resulting solution was extracted with ethyl acetate (3×150 mL), and the organic layers were combined, dried (Na₂SO₄), filtered and concentrated. The residue was purified by column chromatography using a 1:100 ethyl acetate/petroleum ether solvent system to afford 4.3 g (57%) of 1-phenylpyrrolidine as brown oil.

### Synthesis of 4-(pyrrolidin-1-yl) benzenesulfonic acid

A solution of H₂SO₄ (6.8 g, 68.00 mmol) in diethyl ether (80 mL) was added to 1-phenylpyrrolidine (10 g, 68.03 mmol) in diethyl ether (20 mL) at 0° C. The diethyl ether was decanted, and the resulting solution was stirred for 3 h at 170° C., then concentrated in vacuo to afford 7.3 g (43%) of 4-(pyrrolidin-1-yl) benzenesulfonic acid as a white solid.

### Synthesis of 4-(pyrrolidin-1-yl)benzene-1-sulfonyl chloride

DMF (0.5 mL) was added to solution of 4-(pyrrolidin-1-yl)benzenesulfonic acid (7.3 g, 32.16 mmol) in dichloromethane (40 mL). Oxalyl chloride (10 g, 78.74 mmol) was then added dropwise and the resulting solution was maintained at room temperature for 1 h. The reaction mixture was then quenched by the addition of 40 mL of iced water. The resulting solution was extracted using dichloromethane (3×20 mL), and the organic layers were combined, dried (Na₂SO₄), filtered and concentrated. The residue was purified by column chromatography using a 1:100 ethyl acetate/petroleum ether solvent system to afford 1.5 g (19%) of 4-(pyrrolidin-1-yl) benzene-1-sulfonyl chloride as a yellow solid.

¹H NMR (CDCl₃, δ) 0 7.78 (d, 2H), 6.55 (d, 2H), 3.41 (t, 4H), 2.03 (t, 4H).

### Example 13

### Synthesis of 3-(Pyrrolidin-1-yl)benzene-1-sulfonyl Chloride

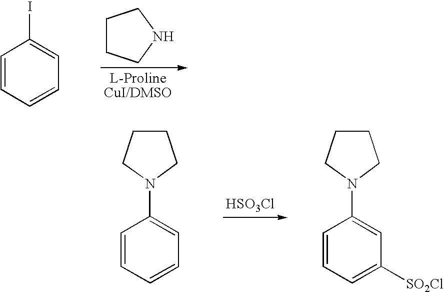

### Synthesis of 1-phenylpyrrolidine

Pyrrolidine (21.6 g, 304.23 mmol), L-proline (1.12 g, 9.74 mmol), and CuI (960 mg, 5.05 mmol) were added sequentially to 1-iodobenzene (10.0 g, 49.02 mmol). Dimethyl sulfoxide (40 mL) was then added, and the resulting solution was stirred at 60° C. for 20 h. The reaction mixture was then quenched by adding 400 mL of iced water. The resulting solution was extracted with ethyl acetate (3×150 mL), and the organic layers were combined, dried (Na₂SO₄), filtered and concentrated. The residue was purified by column chromatography using a 1:100 ethyl acetate/petroleum ether solvent system to afford 4.3 g (57%) of 1-phenylpyrrolidine as brown oil.

### Synthesis of 3-(pyrrolidin-1-yl) benzene-1-sulfonyl chloride

1-Phenylpyrrolidine (4.3 g, 29.25 mmol) was added dropwise to sulfurochloridic acid (20 mL) at 0° C. and the resulting solution was then maintained at 60° C. overnight. The reaction mixture was then quenched by adding 200 mL of ice/salt. The resulting solution was extracted with ethyl acetate (3×100 mL), and the organic layers were combined, dried over Na₂SO₄, filtered and concentrated. The residue was purified by column chromatography using a 1:500 ethyl acetate/petroleum ether solvent system. The collected fractions were combined and concentrated to give 0.5 g (7%) of 3-(pyrrolidin-1-yl)benzene-1-sulfonyl chloride as a yellow solid.

¹H NMR (CDCl₃, δ) 7.36 (m, 1H), 7.24 (d, 1H), 7.07 (s, 1H), 6.82 (d, 1H), 3.34 (t, 4H), 2.05 (t, 4H).

### Example 14

### Preparation of 1-Acetyl-2,3-dihydro-1H-indene-5-sulfonyl Chloride

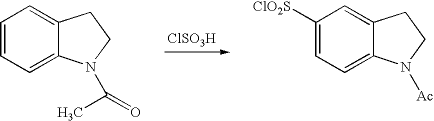

Into a 250 mL 3-necked round-bottom flask, was placed sulfurochloridic acid (16 mL). To the above was added 1-(indolin-1-yl)ethanone (8 g, 49.69 mmol) in several batches, while cooling to a temperature of 0° C. The resulting solution was allowed to react, with stirring, for 45 min while the temperature was maintained at 70° C. in a bath of oil. The reaction progress was monitored by TLC (EtOAc/PE=1:1). The reaction mixture was then quenched by the adding 300 mL of H₂O/ice. A filtration was performed. The filter cake was washed 3 times with 300 mL of water. The filter cake was diluted with 500 mL of dichloromethane. The resulting solution was dried over MgSO4 and concentrated by evaporation under vacuum using a rotary evaporator. This resulted in 5.1 g (36%) of 1-acetylindoline-5-sulfonyl chloride as a light yellow solid.

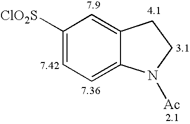

¹H NMR(300 MHz, CDCl₃, δ) 2.1(3H,s), 3.1(2H,t), 4.1(2H,t), 7.36(1H,d), 7.42(1H,d), 7.9(1H,s). [M+H]+ calcd for C₁₁H₁₁ClO₃S+C₇H₉N 329, found 329.

### Example 15

### Preparation of Quinoline-3-sulfonyl Chloride

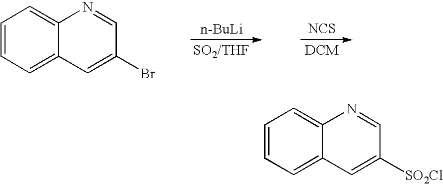

Into a 100 mL 3-necked round-bottom flask purged and maintained with an inert atmosphere of nitrogen, was placed a solution of 3-bromoquinoline (5 g, 24.15 mmol) in THE (50 mL). To the above was added butyllithium (10 mL) dropwise with stirring, while cooling to a temperature of −78° C. The mixture was allowed to react, with stirring, for 40 min at this temperature. Then to the mixture was added SO₂ liquid (2.3 g, 35.94 mmol). The resulting solution was allowed to react, with stirring, for 1 h while warming to room temperature. To the mixture was added hexane. After 30 min, a filtration was performed. The filtrate cake was diluted in dichloromethane. To the above was added NCS (4.8 g, 35.96 mmol) in several batches, while cooling to a temperature of 0° C. The resulting solution was allowed to react, with stirring, for 30 min while the temperature was maintained at room temperature. The reaction progress was monitored by TLC (EtOAc/PE=1:10). The resulting mixture was washed 3 times with 150 mL of NaHCO3 and 3 times with 150 mL of NaCl. The mixture was dried over Na₂SO₄. The residue was purified by eluting through a column with a 1:50 EtOAc/PE solvent system. This resulted in 1.7 g (29%) of quinoline-3-sulfonyl chloride as a yellow solid.

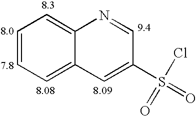

¹H NMR(300 MHz, CDCl₃, δ) 7.8(1H, t), 8.0(1, t), 8.08(1H, d), 8.3(1H, d), 8.9(1H, s), 9.4(1H, s). [M+C₅H₇N₂—Cl]+ calcd for C₁₄H₁₇N₃O₂S 299, found 299.

### Example 16

### Preparation of 2,3-Dihydrobenzofuran-6-sulfonyl Chloride

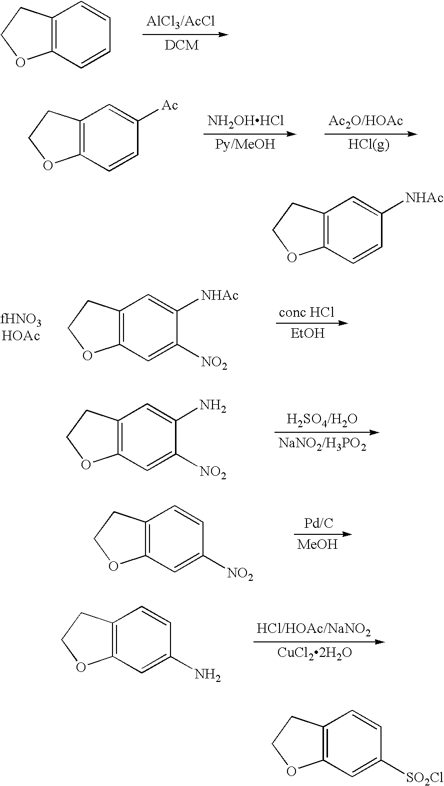

### Preparation of 1-(2,3-dihydrobenzofuran-5-yl)ethanone

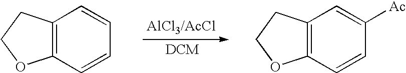

Into a 500 mL 3-necked round-bottom flask, was placed a solution of acetyl chloride (62 g) in dry dichloromethane (400 mL). To this was added aluminum(III) chloride (55.6 g, 1.00 equiv). The mixture was allowed to react, with stirring, for 30 min at −10° C. (solution A). Into another 2000 mL 3-necked round-bottom flask, was placed a solution of 2,3-dibydrobenzofuran (50 g, 0.42 mmol, 1.00 equiv) in dry dichloromethane (500 mL) at −10° C. The solution A was added to the above via a cannula, and was stirred for 30 min at 0° C. The mixture was poured into ice/HCl(5:1 v/v, 1 L). The resulting solution was allowed to react, with stirring, for an additional 2 h while the temperature was maintained at room temperature. The resulting solution was extracted three times with 500 mL of CH₂Cl₂ and dried over Na₂SO₄ and concentrated by evaporation under vacuum using a rotary evaporator. The residue was purified by eluting through a column with a 1:100 EtOAc/PE solvent system. This resulted in 67 g (94%) of 1-(2,3-dihydrobenzofuran-5-yl)ethanone as a yellow solid.

### Preparation of-(2,3-dihydrobenzofuran-5-yl)acetamide

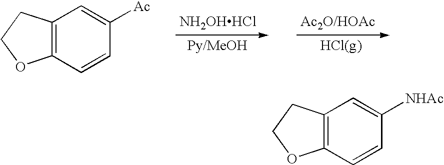

Into a 2000 mL round-bottom flask, was placed a solution of 1-(2,3-dihydrobenzofuran-5-yl)ethanone (67 g, 413.58 mmol, 1.00 equiv) in MeOH (600 mL). To this was added NH₂OH.HCl (34.5 g, 496.40 mmol, 1.20 equiv). To the mixture was added pyridine (Py, 42.5 g, 537.97 mmol, 1.30 equiv). The resulting solution was allowed to react, with stirring, overnight while the temperature was maintained at room temperature. The mixture was concentrated by evaporation under vacuum using a rotary evaporator. The residue was dissolved in 100 mL of water. The resulting solution was extracted two times with 100 mL of EtOAc and the organic layers combined and dried over Na₂SO₄ and concentrated by evaporation under vacuum using a rotary evaporator. This resulted in 70 g (crude) of 1-(2,3-dihydrobenzofuran-5-yl)ethanone oxime. HCl gas was bubbled through a solution of the oxime (70 g) in Ac₂O(86 mL) and HOAc(500 mL). The resulting solution was allowed to react, with stirring, overnight at 20° C. The precipitate was poured into ice/water. The mixture was stirred for 4 h. A filtration was performed. The solid was product (part 1). The filtrate was extracted two times with dichloromethane and was dried over Na₂SO₄ and concentrated. The solid was also product (part 2). Two parts combined and this resulted in 70 g(86%) N-(2,3-dihydrobenzofuran-5-yl)acetamide as a brown oil.

### Preparation of N-(6-nitro-2,3-dihydrobenzofuran-5-yl]acetamide

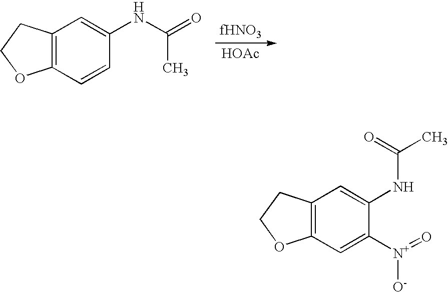

Into a 2000 mL 3-necked round-bottom flask, was placed a solution of N-(2,3-dihydrobenzofuran-5-yl) acetamide (70 g, 395.48 mmol, 1.00 equiv) in HOAc (800 mL). This was followed by the addition of a solution of HNO₃ (fuming) (23 mL, 553.67 mmol, 1.40 equiv) in HOAc (200 mL), which was added dropwise with stirring, while warming to a temperature of 30° C. The resulting solution was allowed to react, with stirring, for 1 h while the temperature was maintained at 15 C in a bath of ice/salt. The reaction progress was monitored by TLC (EtOAc/PE=1:1). The reaction mixture was then quenched by the adding 400 mL of H₂O/ice. A filtration was performed. The filter cake was washed 3 times with 200 mL of water. This resulted in 80 g (91%) of N-(6-nitro-2,3-dihydrobenzofuran-5-yl)acetamide as a yellow solid.

### Preparation of 6-nitro-2,3-dihydrobenzofuran-5-amine

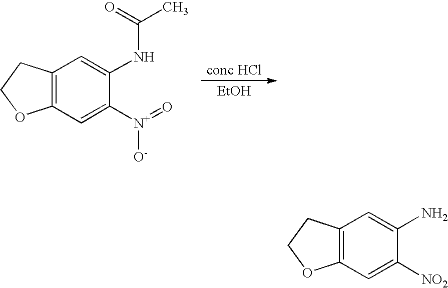

Into a 500 mL round-bottom flask, was placed a solution of N-(6-nitro-2,3-dihydrobenzofuran-5-yl) acetamide (14 g, 63.06 mmol, 1.00 equiv) in EtOH (150 mL). To the mixture was added 6-nitro-2,3-dihydrobenzofuran-5-amine (80 mL). The resulting solution was allowed to react, with stirring, for 1 h while the temperature was maintained at reflux in a bath of oil. The reaction progress was monitored by TLC (EtOAc/PE=1: 1). The reaction mixture was cooled in a bath of ice/salt. Adjustment of the pH to 7 was accomplished by the addition of NH₄OH. A filtration was performed. This resulted in 10 g (88%) of 6-nitro-2,3-dihydrobenzofuran-5-amine as a red solid.

### Preparation of 6-nitro-2,3-dihydrobenzofuran

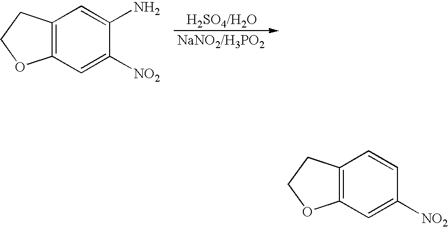

Into a 2000 mL 3-necked round-bottom flask, was placed a solution of 6-nitro-2,3-dihydrobenzofuran-5-amine (57 g, 300.83 mmol, 1.00 equiv, 95%) in H20 (1000 mL). To the mixture was added con H₂SO₄ (570 mL). To the above was added NaNO₂ (24 g, 347.83 mmol, 1.10 equiv) in several batches, while cooling to a temperature of 0° C. To the above was added phosphenous acid (114 mL, 50%) dropwise with stirring, while cooling to a temperature of 0° C. The resulting solution was allowed to react, with stirring, for 1 h while the temperature was maintained at 45° C. in a bath of oil. The reaction progress was monitored by TLC (EtOAc/PE=1:2). The resulting solution was extracted two times with 200 mL of EtOAc and the organic layers combined. The resulting mixture was washed 2 times with 150 mL of water. The mixture was dried over Na₂SO₄ and concentrated by evaporation under vacuum using a rotary evaporator. The residue was purified by eluting through a column with a 1:50 EtOAc/PE solvent system. This resulted in 42 g (76%) of 6-nitro-2,3-dihydrobenzofuran as a red yellow solid.

### Preparation of 2,3-dihydrobenzofuran-6-amine

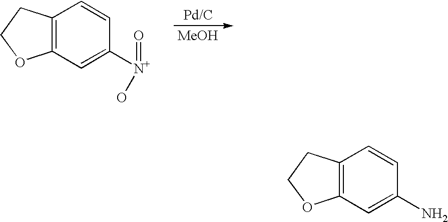

A 1000 mL 3-necked round-bottom flask was purged, flushed and maintained with a hydrogen atmosphere, then, was added a solution of 6-nitro-2,3-dihydrobenzofuran (48 g, 290.91 mmol, 1.00 equiv) in MeOH (800 mL). To the mixture was added Pd/C (10 g). The resulting solution was allowed to react, with stirring, for 3 h while the temperature was maintained at room temperature. The reaction progress was monitored by TLC (EtOAc/PE=1:2). A filtration was performed. The filtrate was concentrated by evaporation under vacuum using a rotary evaporator. This resulted in 37 g (90%) of 2,3-dihydrobenzofuran-6-amine as a yellow solid.

### Preparation of 2,3-dihydrobenzofuran-6-sulfonyl chloride

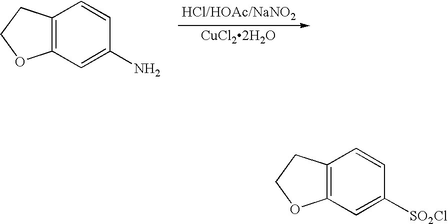

Into a 1000 mL 3-necked round-bottom flask, was placed a solution of 2,3-dihydrobenzofuran-6-amine (30 g, 222.22 mmol, 1.00 equiv) in CH3CN (500 mL). To the mixture was added HCl/HOAc (180/120 g), while cooling to a temperature of 0° C. To the above was added NaNO₂ (18.5 g, 268.12 mmol, 1.20 equiv) in several batches, while cooling to a temperature of 0° C. The resulting solution was allowed to react, with stirring, for 30 min while the temperature was maintained at 0° C. in a bath of ice/salt. To the above was added CuCl₂.2H₂O (41.7 g, 244.57 mmol, 1.10 equiv) in several batches, while cooling to a temperature of 0° C. Then SO₂ gas was inputted to the mixture for 2 h. To the above was added CuCl₂.2H₂O (6.95 g, 40.76 mmol, 1.10 equiv) in several batches, while cooling to a temperature of 0° C. and the SO₂ gas bubbled for another 2 h at 0° C. The solution was reacted with stirring, overnight at room temperature. The reaction progress was monitored by TLC (EtOAc/PE=1:2). The reaction mixture was then quenched by the adding 600 mL of H₂O/ice. The resulting solution was extracted three times with 500 mL of EtOAc and the organic layers combined. The resulting mixture was washed 2 times with 400 mL of water. The mixture was dried over Na₂SO₄. The residue was purified by eluting through a column with a 1:20 EtOAc/PE solvent system and was washed with hexane. This resulted in 26.2 g (54%) of 2,3-dihydrobenzofuran-6-sulfonyl chloride as a white solid.

LC-MS-(ES, m/z): [M+H+C₅H₁₂N₂—Cl]+ calcd for C₁₃H₁₉N₂O3S 283, found 283

¹H NMR (CDCl₃, 300 MHz, δ) 3.2(2H,m), 4.7(2H,m), 7.55(1H,s), 7.37-7.39(2H,d)

### Example 17

### Preparation of (S)-4-(3-Methoxypyrrolidin-1-yl)benzene-1-sulfonyl Chloride

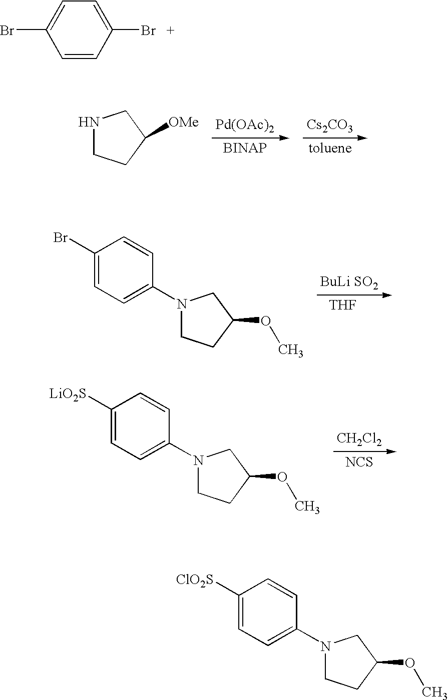

### Synthesis of (S)-1-(4-bromophenyl)-3-methoxypyrrolidine

Into a 250 mL 3-necked round-bottom flask purged and maintained with an inert atmosphere of nitrogen, was placed a solution of 1,4-dibromobenzene (10 g, 42.37 mmol) in toluene (100 mL). To this was added (S)-3-methoxypyrrolidine (5.14 g, 50.89 mmol). Addition of Cs₂CO₃ (34 g, 104.29 mmol) was next. This was followed by the addition of BINAP (800 mg, 1.28 mmol). To the mixture was added Pd(OAc)₂ (95 mg, 0.42 mmol). The resulting solution was allowed to react, with stirring, overnight while the temperature was maintained at 120° C. in a bath of oil. The reaction progress was monitored by TLC (EtOAc/PE=1:8). A filtration was performed. The filtrate was concentrated by evaporation under vacuum using a rotary evaporation. The residue was purified by eluting through a column with a 1:100 EtOAc/PE solvent system. This resulted in 4.8 g (44%) of (S)-1-(4-bromophenyl)-3-methoxypyrrolidine as a yellow solid.

### Synthesis of lithium 4-((S)-3-methoxpyrrolidin-1-yl)benzenesulfinate

Into a 500 mL 3-necked round-bottom flask purged and maintained with an inert atmosphere of nitrogen, was placed a solution of (S)-1-(4-bromophenyl)-3-methoxypyrrolidine (4.8 g, 18.75 mmol) in THF (60 mL). To the above was added BuLi (9 mL) dropwise with stirring, while cooling to a temperature of −78° C., and the resulting solution was allowed to react, with stirring, for 1 h at −78° C., then SO₂ (2 mL) was added dropwise to the above mixture. Then the resulting solution was allowed to react, with stirring, for an additional 4 h while the temperature was maintained at room temperature. The product was precipitated by the addition of hexane (50 mL). A filtration was performed. The filter cake was washed 2 times with 10 mL of CH₂Cl₂. This resulted in 5 g (50%) of lithium 4-((S)-3-methoxypyrrolidin-1-yl)benzenesulfinate as a yellow solid.

### Synthesis of (S)-4-(3-methoxypyrrolidin-1-yl)benzene-1-sulfonyl chloride

Into a 250 mL round-bottom flask, was placed a solution of lithium 4-((S)-3-methoxypyrrolidin-1-yl)benzenesulfinate (5 g, 9.31 mmol) in CH₂Cl₂ (100 mL). To the above was added 1-chloropyrrolidine-2,5-dione (1.87 g, 14.01 mmol) in several batches, while cooling to a temperature of 0° C. over a time period of 15 min. The resulting solution was allowed to react, with stirring, for 1 h while the temperature was maintained at room temperature. The reaction progress was monitored by TLC (EtOAc/PE=1:1). The reaction mixture was then quenched by the adding 100 mL of NaHSO₃ (sat). The organic layer was washed 2 times with 50 mL of brine. The mixture was concentrated by evaporation under vacuum using a rotary evaporator. The residue was purified by eluting through a column with a 2:3 EtOAc/PE solvent system. This resulted in 2 g(77%) of (S)-4-(3-methoxypyrrolidin-1-yl)benzene-1-sulfonyl chloride as a yellow solid.

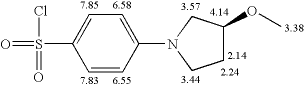

¹H NMR (300 Hz, CDCl₃, δ) 2.14-2.10(1H,m), 3.38(3H,s) □3.57-3.44 (4H,m) □4.14 (1H,s), 6.58(1H,d,J=9 Hz), 6.55(1H,d, J=9 Hz), 7.83(1H,d, J=9 Hz), 7.85(1H,d, J=9 Hz)

LCMS [M+BnNH−H]⁻ calcd for C₁₈H₂₁N₂O₃S 345 found 345

### Example 18

### Preparation of 2-Oxo-1,2-dihydroquinoline-6-sulfonyl Chloride

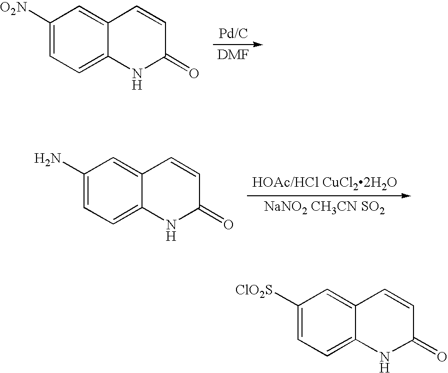

### Preparation of 6-aminoquinolin-2(1H)-one

Into a 500 mL 3-necked round-bottom flask purged and maintained with an inert atmosphere of nitrogen, was placed a solution of 6-nitroquinolin-2(1H)-one (10 g, 52.58 mmol, 1.00 equiv) in DMF (200 mL). To the mixture was added Pd/C (8.6 g). The resulting solution was allowed to react, with stirring, overnight while the temperature was maintained at room temperature under H₂ gas. The reaction progress was monitored by TLC (MeOH/DCM=1:10). A filtration was performed. The filtrate was concentrated by evaporation. The resulting mixture was washed one times with 100 mL of H₂O and one times with 10 mL of n-hexane. A filtration was performed. The filter cake was washed one time with 100 mL of H₂O and one time with 10 mL of n-hexane. This resulted in 8 g (90%) of 6-aminoquinolin-2(1H)-one as a gray solid.

### Preparation of 2-oxo-1,2-dihydroquinoline-6-sulfonyl chloride

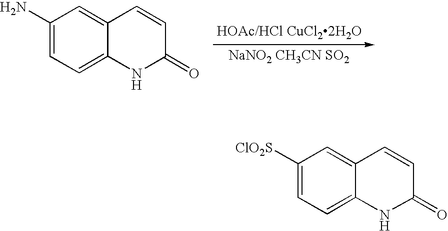

Into a 250 mL 3-necked round-bottom flask, was placed a solution of 6-aminoquinolin-2(1H)-one(2 g, 12 mmol, 1.00 equiv) in CH₃CN (150 mL). To this was added HOAc (15 g). To the mixture was added HCl (6.5g, 36%). This was followed by the addition of a solution of NaNO₂ (1.1 g, 16 mmol, 1.20 equiv) in H₂O (1 mL) in several batches, while cooling to a temperature of −5-0° C. The resulting solution was allowed to react, with stirring, for 30 min while the temperature was maintained at −5-0° C. in a bath of H₂O/ice. This was followed by and maintained with an atmosphere of sulfur dioxide. The resulting solution was allowed to react, with stirring, for an additional 2 h while the temperature was maintained at −5to 0° C. in a bath of H₂O/ice. This was followed by the addition of a solution of CuCl₂.2H₂O (1.01 g, 12.9 mmol, 1.00 equiv) in H₂O, which was added dropwise with stirring, while cooling to a temperature of −5 to 0° C. The resulting solution was allowed to react, with stirring, for 2 h while the inert atmosphere was maintained with SO₂ gas. The resulting solution was allowed to react, with stirring, overnight while the temperature was maintained at room temperature. The reaction progress was monitored by TLC (EtOAc/PE=1:10). The reaction mixture was then quenched by the adding 100 mL of H₂O/ice. The resulting solution was extracted two times with 1000 mL of CH₂Cl₂ and the organic layers combined and dried over Na₂SO₄ and concentrated by evaporation under vacuum using a rotary evaporator. The resulting mixture was washed one time with 10 mL of n-hexane. This resulted in 0.12 g (4%) of 2-oxo-1,2-dihydroquinoline-6-sulfonyl chloride as a gray solid.

LC-MS (ES, m/z): [M+C₅H₁₁N₂+H—Cl]+ calcd for C₁₄H₁₇N₃O₃S 308, found 308

¹H NMR-(300 MHz, CDCl₃, δ) 6.48(1H, d), 7.25(1H, d), 7.72(1H, d), 7.95(2H, m), 11.80(1H, s)

### Example 19

### Preparation of (S)-5-(3-Methoxypyrrolidin-1-yl)pyridine-3-sulfonyl Chloride

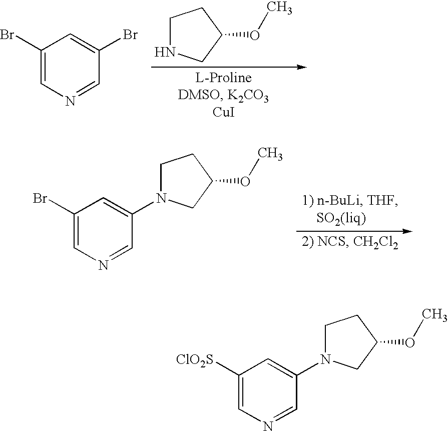

### Synthesis of (S)-3-bromo-5-(3-methoxypyrrolidin-1-yl)pyridine

Into a 150 mL sealed tube purged and maintained with an inert atmosphere of nitrogen, was placed a solution of 3,5-dibromopyridine (10 g, 42.19 mmol) in DMSO (50 mL). To this was added (S)-3-methoxypyrrolidine (5.1 g, 50.50 mmol). Addition of L-proline (970 mg, 8.43 mmol) was next. This was followed by the addition of CuI (800 mg, 4.21 mmol). To the mixture was added K₂CO₃ (11.6 g, 84.06 mmol). The resulting solution was allowed to react, with stirring, for 40 h while the temperature was maintained at 90° C. A filtration was performed. The resulting solution was diluted with 100 mL of H₂O. The resulting solution was extracted three times with 100 mL of EtOAc and the organic layers combined. The resulting mixture was washed 5 times with 100 mL of brine. The mixture was dried over Na₂SO₄. A filtration was performed. The filtrate was concentrated by evaporation under vacuum using a rotary evaporator. The residue was purified by eluting through a column with a 1:10 EtOAc/PE solvent system. This resulted in 1.8 g (17%) of (S)-3-bromo-5-(3-methoxypyrrolidin-1-yl)pyridine as yellow oil.

### Synthesis of (S)-5-(3-methoxypyrrolidin-1-yl)pyridine-3-sulfonyl chloride

Into a 100 mL 3-necked round-bottom flask purged and maintained with an inert atmosphere of nitrogen, was placed a solution of (S)-3-bromo-5-(3-methoxypyrrolidin-1-yl)pyridine (1.8 g, 7.00 mmol) in THF (30 mL). To the above was added n-BuLi (3.4 mL) dropwise with stirring, while cooling to a temperature of −78° C. Then the mixture was stirred for 30 min at −78° C. To the above was added SO₂ (490 mg, 7.66 mmol) dropwise with stirring, while cooling to a temperature of −78° C. Then the mixture was reacted at room temperature overnight. To the mixture 50 mL of hexane was added. The resulting mixture was filtrated and the filter cake was suspended in 30 mL of CH₂Cl₂. To the above was added NCS (1.39 g, 10.41 mmol) in several batches. The resulting solution was allowed to react, with stirring, for 1 h while the temperature was maintained at room temperature. The resulting solution was diluted with 30 mL of CH₂Cl₂ The resulting mixture was washed 2 times with 50 mL of 2M NaHSO₃ and 3 times with 50 mL of brine. The mixture was dried over Na₂SO₄. A filtration was performed. The filtrate was concentrated by evaporation under vacuum using a rotary evaporator. The residue was purified by cluting through a column with a 1:5 EtOAc/PE solvent system. This resulted in 0.38 g (20%) of (S)-5-(3-methoxypyrrolidin-1-yl)pyridine-3-sulfonyl chloride as yellow oil.

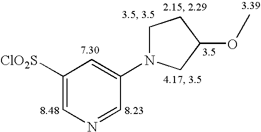

¹H NMR (400 MHz, CDCl₃ δ) 2.15(1H, m), 2.29 (1H, m), 3.39 (3H, s), 3.45-3.56 (4H, m), 4.17 (1H, s), 7.30 (1H, s), 8.23 (1H, s) 8.48 (1H, s).

LC-MS(436-166)-06031 7PM

[M+H+BnNH]+ calcd for C₁₇H₂₂N₃O₃S 348,found 348.

### Example 20

### Preparation of 4-(Dimethylamino)benzene-1-sulfonyl Chloride

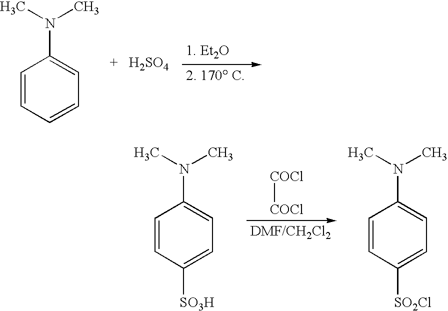

### Synthesis of 4-(dimethylamino)benzenesulfonic acid

Into a 250 mL 3-necked round-bottom flask, was placed a solution of N,N-dimethylbenzenamine (20 g, 165.29 mmol) in Et₂O (40 mL) in the ice bath. This was followed by the addition of a solution of H₂SO₄ (16.1 g, 161.00 mmol) in Et₂O (160 mL). Then the Et₂O was removed out. The resulting solution was allowed to react, with stirring, for 4 h while the temperature was maintained at 170° C. in a vacuum. This resulted in 10.5 g (32%) of 4-(dimethylamino)benzenesulfonic acid as a white solid.

### Synthesis of 4-(dimethylamino)benzene-1-sulfonyl chloride

Into a 500 mL round-bottom flask, was placed 4-(dimethylamino) benzenesulfonic acid (10 g, 49.75 mmol). To this was added CH₂Cl₂ (200 mL). To the mixture was added DMF (4 mL). To the above was added dropwise oxalyl dichloride (25 g, 196.85 mmol). The resulting solution was allowed to react with stirring for 0.5 h at room temperature. The reaction progress was monitored by TLC (EtOAc/PE=1:2). The reaction mixture was then quenched by the adding 200 mL of ice/salt. The resulting solution was extracted twice with 50 mL of CH₂Cl₂ and the organic layers combined and dried over Na₂SO₄ A filtration was performed. The filtrate was concentrated by evaporation under vacuum using a rotary evaporator. This resulted in 9.1 g (53%) of 4-(dimethylamino)benzene-1-sulfonyl chloride as a yellow solid

¹H NMR: (CDCl₃, δ) 7.84(d,2H), 6.71 (d,2H), 3.12(s,6H).

### Example 21

### Preparation of 2,3-Dihydrobenzofuran-4-sulfonyl Chloride

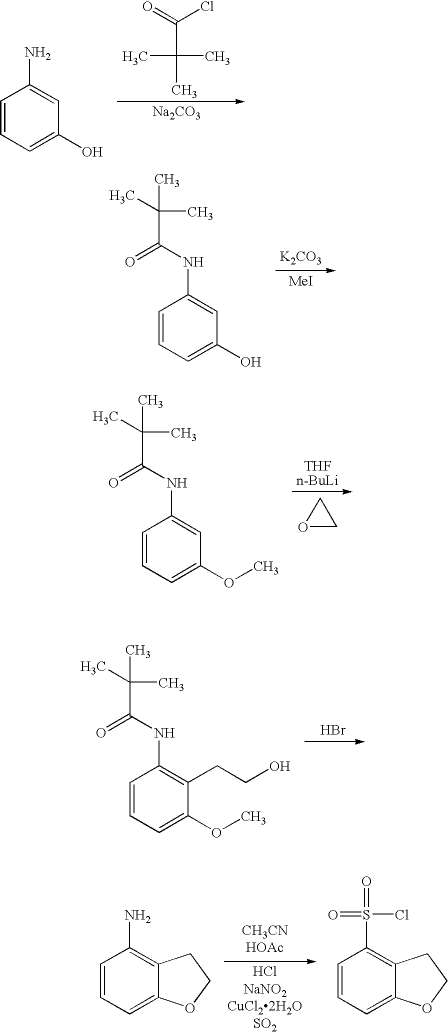

### Synthesis of N-(3-hydroxyphenyl)pivalamide

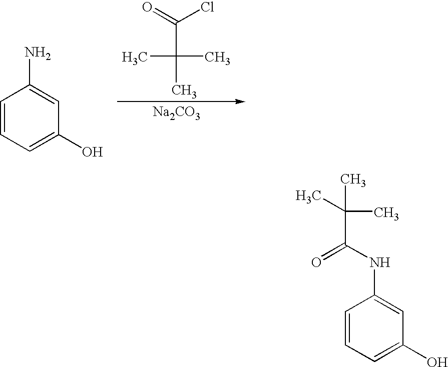

Into a 500 mL 3-necked round-bottom flask, was placed a solution of 3-aminophenol (3.98 g, 36.51 mmol, 1.00 equiv) in EtOAc (125 mL). This was followed by the addition of a solution of Na₂CO₃ (9.2 g, 86.79 mmol, 3.00 equiv) in H₂O (150 mL). To the above was added pivaloyl chloride (4.62 g, 38.31 mmol, 1.10 equiv) dropwise with stirring while the temperature was maintained at 0° C. in a bath of H₂O/ice. The resulting solution was allowed to react, with stirring, for 1 h. The reaction progress was monitored by TLC (EtOAc/PE=1:2). The resulting organic phase was washed with HCl(1N), H₂O and brine. The organic phase was dried over Na₂SO₄ and concentrated by evaporation under vacuum using a rotary evaporator. This resulted in 6.7 g (90%) of N-(3-hydroxyphenyl)pivalamide as a gray solid.

### Synthesis of N-(3-methoxyphenyl)pivalamide

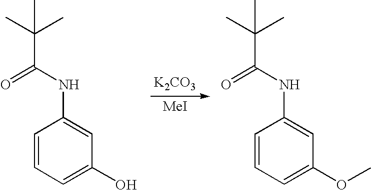

Into a 1000 mL round-bottom flask, was placed a solution of N-(3-hydroxyphenyl)pivalamide (13.4 g, 69.43 mmol, 1.00 equiv) in acetone (500 mL). To this was added K₂CO₃ (28.5 g, 206.52 mmol, 3.00 equiv). To the mixture was added MeI (39.4 g, 277.46 mmol, 4.00 equiv). The resulting solution was allowed to react, with stirring, for 3 h while the temperature was maintained at reflux in a bath of oil. The reaction progress was monitored by TLC (EtOAc/PE=1.2). A filtration was performed. The filtrate was concentrated by evaporation under vacuum using a rotary evaporator. The resulting mixture was washed with hexane. A filtration was performed. This resulted in 13.9 g (91%) of N-(3-methoxyphenyl)pivalamide as a white solid.

### Synthesis of N-(2-(2-hydroxyethyl)-3-methoxyphenyl)pivalamide

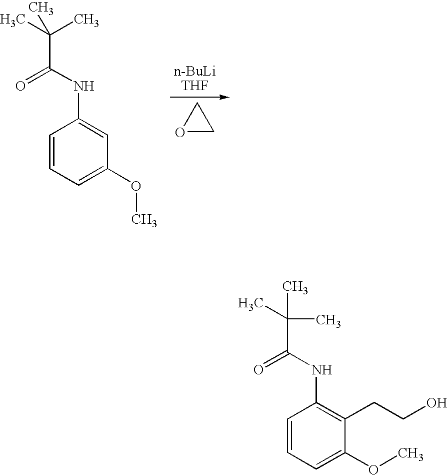

Into a 250 mL 3-necked round-bottom flask purged and maintained with an inert atmosphere of nitrogen, was placed a solution of N-(3-methoxyphenyl)pivalamide (11.8 g, 57.00 mmol, 1.00 equiv) in THE (200 mL). To the above was added n-BuLi (60 mL) dropwise with stirring while the temperature was maintained at 0° C. in a bath of H₂O/ice. The resulting solution was allowed to react with stirring, for 2 h. To the above was added oxirane (7 mL, 1.50 equiv) dropwise with stirring while the temperature was maintained at 0° C. in a bath of H₂O/ice. The resulting solution was allowed to react with stirring, for 1 h while the temperature was maintained at 0° C. in a bath of H₂O/ice. The resulting solution was allowed to react for 2 h while the temperature was maintained at room temperature. The reaction mixture was then quenched by the adding H₂O. The mixture was concentrated by evaporation under vacuum using a rotary evaporator. The resulting solution was extracted with EtOAc and the organic layers combined. The organic phase was washed with Na₂CO₃. The mixture was dried over Na₂SO₄ and concentrated by evaporation under vacuum using a rotary evaporator. The final product was purified by recrystallization from DCM/hexane. This resulted in 10.5 g (53%) of N-(2-(2-hydroxyethyl)-3-methoxyphenyl)pivalamide as a white solid.

### Synthesis of 2,3-dihydrobenzofuran-4-amine

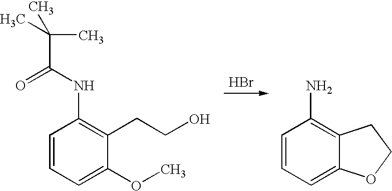

Into a 210 mL sealed tube purged and maintained with an inert atmosphere of nitrogen, was placed N-(2-(2-hydroxyethyl)-3-methoxyphenyl)pivalamide (10.5 g, 41.83 mmol, 1.00 equiv). To the mixture was added HBr (48%) (100 mL). The resulting solution was allowed to react, with stirring, overnight while the temperature was maintained at 100° C. in a bath of oil. The reaction progress was monitored by TLC (EtOAc/PE=1:2). Adjustment of the pH to 9 was accomplished by the addition of NaOH. The resulting solution was extracted with EtOAc and the organic layers combined. The resulting mixture was washed with H₂O. The mixture was dried over Na₂SO₄ and concentrated by evaporation under vacuum using a rotary evaporator This resulted in 2.5 g (40%) of 2,3-dihydrobenzofuran-4-amine as yellow oil.

### Synthesis of 2,3-dihydrobenzofuran-4-sulfonyl chloride

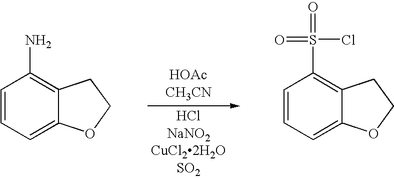

Into a 250 mL 3-necked round-bottom flask, was placed a solution of 2,3-dihydrobenzofuran-4-amine (2.2 g, 16.30 mmol, 1.00 equiv) in CHCN (200 mL). To the above was added HOAc (9 g) dropwise with stirring, while cooling to a temperature of 0° C. To the above was added HCl (9 g) dropwise with stirring, while cooling to a temperature of 0° C. This was followed by the addition of a solution of NaNO₂ (1.52 g, 22.03 mmol, 1.50 equiv) in H₂O (2 mL), which was added dropwise with stirring, while cooling to a temperature of 0° C. The mixture was stirred for 30 min and was bubbled SO₂ for 2 h, while cooling to a temperature of 0° C. This was followed by the addition of a solution of CuCl₂.2H₂O (3.4 g, 20.00 mmol, 1.20 equiv) in H₂O (3 mL), which was add stirring. The resulting solution was allowed to react, with stirring, overnight while the temperature was maintained at 15° C. in a bath of oil. The reaction progress was monitored by TLC (EtOAc/PE=1:2). The reaction mixture was then quenched by the adding of H₂O/ice. The resulting solution was extracted one time with of EtOAc and the organic layers combined. The resulting mixture was washed with H₂O. The mixture was dried over Na₂SO₄ and concentrated by evaporation under vacuum using a rotary evaporator. The residue was purified by eluting through a column with a 1:70 EtOAc/PE solvent system. This resulted in 1.42 g (40%) of 2,3-dihydrobenzofuran-4-sulfonyl chloride as a yellow solid.

LC-MS (ES, m/z): [M+C₅H₁₁N₂-Cl+H]+ calcd for C₁₃H₁₉N₂O₃S 283, found 283

¹H NMR (300 MHz, CDCl₃, δ) 7.4(d, 1H)7.3(d, 1H),7.1(d, 1H),4.7(m,2H),3.6(m,2H).

### Example 22

### Preparation of 2,3-Dihydrobenzofuran-7-sulfonyl Chloride

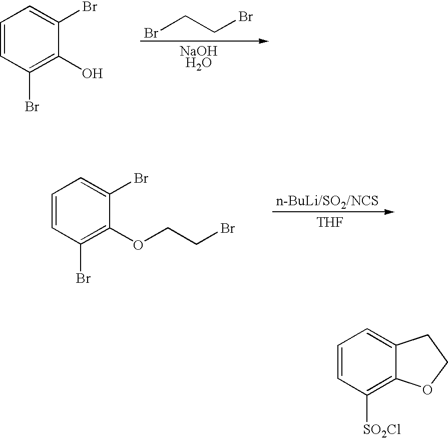

### Synthesis of 1,3-dibromo-2-(2-bromoethoxy benzene

Into a 100 mL 3-necked round-bottom flask, was placed a solution of 2,6-dibromophenol (14.5 g, 57.54 mmol, 1.00 equiv) in H₂O (45 mL). To the mixture was added NaOH (2.5 g, 62.50 mmol 1.10 equiv). To the above was added 1,2-dibromoethane (5 mL, 1.00 equiv) dropwise with stirring. The resulting solution was allowed to react, with stirring, for 17 h while the temperature was maintained at reflux in a bath of oil. The reaction progress was monitored by TLC (EtOAc/PE=1:10). The resulting solution was extracted two times with 100 mL of diethyl ether and the organic layers combined. The resulting mixture was washed 1 time with 100 mL of NaOH(1M) and 1 time with 100 mL of brine. The mixture was dried over Na₂SO₄ and concentrated by evaporation under vacuum using a rotary evaporator. The residue was purified by eluting through a column with a 1:1000 EtOAc/PE solvent system. This resulted in 14.5 g (69%) of 1,3-dibromo-2-(2-bromoethoxy)benzene as a colorless liquid.

### Synthesis of 2,3-dihydrobenzofuran-7-sulfonyl chloride

Into a 250 mL 3-necked round-bottom flask purged and maintained with an inert atmosphere of nitrogen, was placed a solution of 1,3-dibromo-2-(2-bromoethoxy)benzene (8 g, 21.84 mmol, 1.00 equiv, 98%) in TVT (100 mL). To the above was added n-BuLi (8 mL, 1.00 equiv, 2.9M) dropwise with stirring, while cooling to a temperature of −100° C. The resulting solution was reacted with stirring for 30 mins while the temperature was maintained at −100° C. Then to the above was added n-BuLi (8 mL, 1.00 equiv, 2.9M) dropwise with stirring, while cooling to a temperature of −100° C. Then the mixture was stirred for 1 h. To the mixture was added SO₂ (2.8 g, 43.75 mmol, 2.00 equiv), while cooling to a temperature of −85-−100° C. The resulting solution was allowed to react, with stirring, for another 2 h. To the above was added hexane (100 mL) until the solid appeared. A filtration was performed the filter cake was dissolved in 100 mL dichloromethane after filtration. Then added NCS (3.3 g, 24.63 mmol, 1.10 equiv) in several batches, while cooling to a temperature of 0° C. The resulting solution was allowed to react, with stirring, for 1 h while the temperature was maintained at 0° C. in a bath of H₂O/ice. The reaction progress was monitored by TLC (EtOAc/PE=1:5). The resulting solution was diluted with 100 mL of CH₂Cl₂. The resulting mixture was washed 2 times with 150 mL of NaHSO₃ and 3 times with 100 mL of brine. The mixture was dried over Na₂SO₄ and concentrated by evaporation under vacuum using a rotary evaporator. The residue was purified by eluting through a column with a 1:50 EtOAc/PE solvent system. This resulted in 2.5 g (51%) of 2,3-dihydrobenzofuran-7-sulfonyl chloride as a light yellow solid.

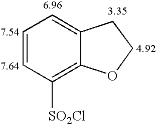

¹H NMR(300 MHz, CDCl3, δ) 3.35(2H,t), 4.92(2H,t), 6.96(1H, t), 7.54(1H,s), 7.64(1H,d)

LC-MS (ES, m/z):[C₁₃H₁₈N₂O₃S+H]+ calcd for C₁₃H₁₉N₂O₃S 283, found 283.

### Example 23

### Preparation of 3-Oxo-3,4-dihydro-2H-benzo[b][1,4]oxazine-5-sulfonyl Chloride

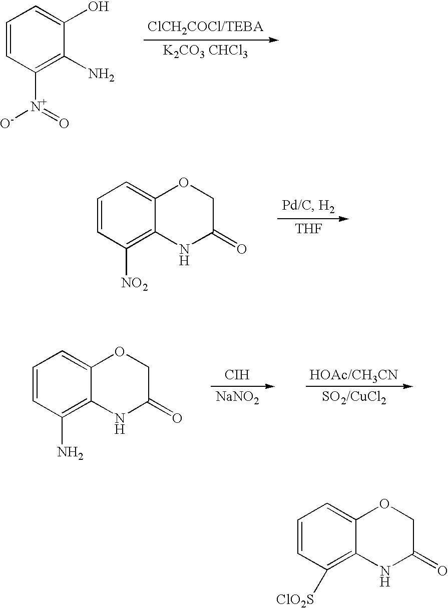

### Synthesis of 5-nitro-2H-benzo[b][1,4]oxazin-3 (4H)-one

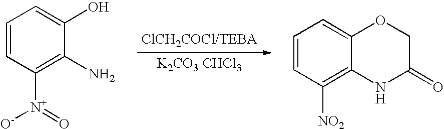

Into a 2000 mL 3-necked round-bottom flask, was placed a solution of 2-amino-3-nitrophenol (20 g, 129.87 mmol, 1.00 equiv) in CHCl₃ (800 ML). To this was added TEBA (29.6 g, 129.82 mmol, 1.00 equiv). To the mixture was added K₂CO₃ (53.76 g, 389.57 mmol, 3.00 equiv). This was followed by the addition of a solution of 2-chloroacetyl chloride (17.6 g, 155.75 mmol, 1.20 equiv) in CHCl₃ (200 mL), which was added dropwise with stirring, while cooling to a temperature of 0-5° C. over a time period of 45 min. The resulting solution was allowed to react, with stirring, for 1 h while the temperature was maintained at 0-5° C. in a bath of H₂O/ice. The reaction progress was monitored by TLC (EtOAc:PE=1:2). Then the resulting solution was allowed to react, with stirring, overnight while the temperature was maintained at 65° C. in a bath of oil. A filtration was performed. The filtrate was concentrated by evaporation under vacuum using a rotary evaporator. The product was precipitated by the addition of H₂O. A filtration was performed. The filter cake was washed 3 times with 200 mL of H₂O. The final product was purified by recrystallization from EtOH. This resulted in 18.0 g (64%) of 5-nitro-2H-benzo[b][1,4]oxazin-3(4H)-one as a yellow solid.

### Synthesis of 5-amino-2H-benzo[b][1,4]oxazin-3 (4H)-one

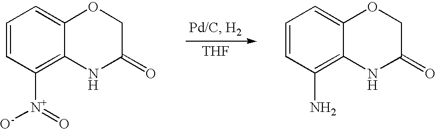

A 500 mL 3-necked round-bottom flask was purged, flushed and maintained with a hydrogen atmosphere, then, was added a solution of 5-nitro-2H-benzo[b][1,4]oxazin-3(4H)-one (7.0 g, 32.47 mmol, 1.00 equiv, 90%) in THE (300 mL). To the mixture was added Pd/C (3 g). The resulting solution was allowed to react, with stirring, overnight while the temperature was maintained at 25° C. The reaction progress was monitored by TLC (PE/EtOAc=2:1). A filtration was performed. The filtrate was concentrated by evaporation under vacuum using a rotary evaporator. The product was precipitated by the addition of H₂O. A filtration was performed. The filter cake was washed 3 times with 100 mL of H₂O and 3 times with 100 mL of ether. This resulted in 6.0 g (100%) of 5-amino-2H-benzo[b][1,4]oxazin-3(4H)-one as a light yellow solid.

### Synthesis of 3-oxo-3,4-dihydro-2H-benzo[b][1,4]oxazine-5-sulfonyl chloride

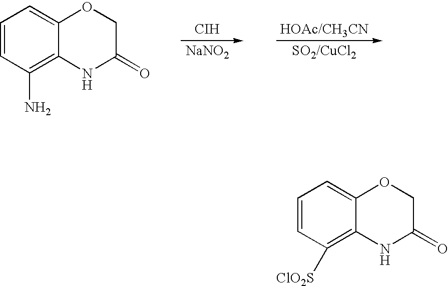

Into a 500 ml 3-necked round-bottom flask, was placed a solution of 5-amino-2H-benzo[b][1,4]oxazin-3(4H)-one (5 g, 28.96 mmol, 1.00 equiv 95%) in CH₃CN (300 mL). To the above was added HOAc (24.9 g) dropwise with stirring, while cooling to a temperature of 0° C. To the above was added HCl (16.2 g, 36.5%) dropwise with stirring, while cooling to a temperature of 0° C. This was followed by the addition of a solution of NaNO₂ (2.52 g, 36.52 mmol, 1.20 equiv) in H₂O (2 mL), which was added dropwise with stirring, while cooling to a temperature of 0° C. The resulting solution was allowed to react with stirring, for 30 min while the temperature was maintained at 0 to 5° C. in a bath of H₂O/ice. This was followed by and maintained with an atmosphere of sulfur dioxide, the resulting solution was allowed to react, with stirring, for an additional 2 h while the temperature was maintained at 0-−5° C. in a bath of H₂O/ice. To the mixture was added CuCl₂.2H₂O (5.11 g, 29.97 mmol, while cooling to a temperature of 0 to 5° C. The resulting solution was allowed to react, with stirring, maintained with an atmosphere of sulfur dioxide for an additional 2 h while the temperature was maintained at 0-−5° C. in a bath of H₂O/ice. The resulting solution was allowed to react, with stirring, overnight while the temperature was maintained at 25° C. The reaction progress was monitored by TLC (PE:EtOAc=1:1). The reaction mixture was then quenched by the adding 200 mL of H₂O/ice. The resulting solution was extracted 3 times with 300 mL of dichloromethane and the organic layers combined. The resulting mixture was washed 5 times with 200 mL of brine. The mixture was dried over MgSO₄. The residue was purified by eluting through a column with a 1:15 EtOAc/PE solvent system. This resulted in 0.9 g (11%) of 3-oxo-3,4-dihydro-2H-benzo[b][1,4]oxazine-5-sulfonyl chloride as a light yellow solid.

LC-MS (ES, m/z): [M+C₅H₁₁N₂-Cl]+ calcd for C₁₃H₁₇N₃O₄S 312, found 312

¹H NMR (CDCl₃, 300 MHz, δ): 9.06(1H,s), 7.69(1H,d), 7.36(1H,m), 7.18(1H,d), 4.75(2H),s)

### Example 24

### Preparation of 3-Oxo-3,4-dihydro-2H-benzo[b][1,4]oxazine-8-sulfonyl Chloride

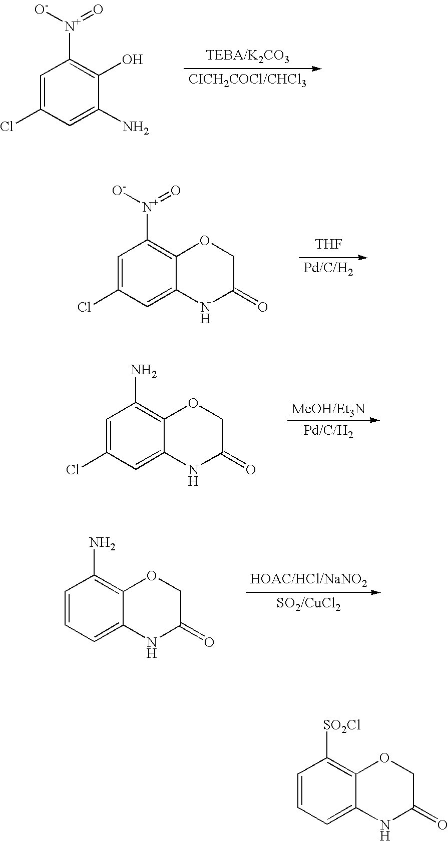

### Synthesis of 6-chloro-8-nitro-2H-benzo[b][1,4]oxazin-3 (4H)-one

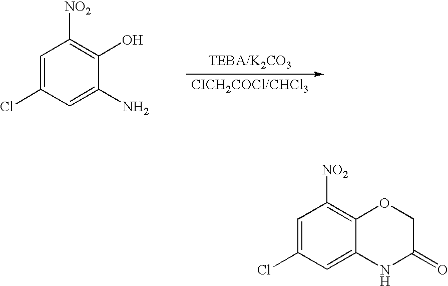

Into a 5000 mL 3-necked round-bottom flask, was placed a solution of 2-amino-4-chloro-6-nitrophenol (40 g, 212.09 mmol, 1.00 equiv) in CHCl₃ (2500 mL). To this was added N-benzyl-N-chloro-N,N-diethylethanamine (TEBA, 48 g, 210.53 mmol, 1.00 equiv). To the mixture was added K₂CO₃ (88 g, 637.68 mmol, 3.00 equiv). This was followed by the addition of a solution of 2-chloroacetyl chloride (28.8 g, 254.87 mmol, 1.20 equiv) in CHCl₃ (500 mL), which was added dropwise with stirring, while cooling to a temperature of 0-5° C. The resulting solution was allowed to react, with stirring, for 1 h while the temperature was maintained at 0-5° C. in a bath of ice/salt. The reaction progress was monitored by TLC (EtOAc/PE=1:5). The resulting solution was allowed to react, with stirring, overnight while the temperature was maintained at 55° C. in a bath of oil. The reaction progress was monitored by TLC (EtOAc/PE=1:5). A filtration was performed. The filtrate was concentrated by evaporation under vacuum using a rotary evaporator. The resulting solution was diluted with 500 mL of H₂O. A filtration was performed. The final product was purified by recrystallization from EtOH. This resulted in 34.7 g (72%) of 6-chloro-8-nitro-2H-benzo[b][1,4oxazin-3(4H)-one as a brown solid.

### Synthesis of 8-amino-6-chloro-2H-benzo[b][1,4]oxazin-3(4H)-one

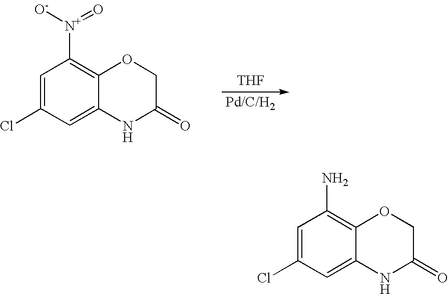

A 1000 mL 3-necked round-bottom flask was purged, flushed and maintained with a hydrogen atmosphere, then, was added a solution of 6-chloro-8-nitro-2H-benzo[b][1,4]oxazin-3(4-H)-one (8 g, 35.00 mmol, 1.00 equiv) in THE (700 mL). To the mixture was added Pd/C (3 g). The resulting solution was allowed to react, with stirring, for 4 h while the temperature was maintained at 35° C. in a bath of oil. The reaction progress was monitored by TLC (EtOAc/PE=1:1). A filtration was performed. The filtrate was concentrated by evaporation under vacuum using a rotary evaporator. This resulted in 6.7 g (92%) of 8-amino-6-chloro-2H-benzo[b][1,4]oxazin-3(4H)-one as a brown solid.

### Synthesis of 8-amino-2H-benzo[b][1,4]oxazin-3(4H)-one

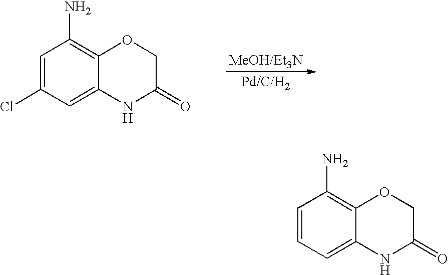

A 250 mL round-bottom flask was purged, flushed and maintained with a hydrogen atmosphere, then, was added a solution of 8-amino-6-chloro-2H-benzo[b][1,4]oxazin-3(4H)-one (2 g, 9.57 mmol, 1.00 equiv, 95%) in MeOH (50 mL). To the mixture was added triethylamine (3 g, 29.70 mmol, 3 equiv). The resulting solution was allowed to react, with stirring, for 3 h while the temperature was maintained at room temperature ° C. in a bath of oil. The reaction progress was monitored by TLC (EtOAc/PE=1:1). A filtration was performed. The filtrate was concentrated by evaporation under vacuum using a rotary evaporator. This resulted in 1 g (64%) of 8-amino-2H-benzo[b][1,4]oxazin-3(4H)-one as a white solid.

¹H NMR (DMSO, 300 MHz, δ) 10.46(1H,s), 6.63(1H,m), 6.33(1H,d), 6.13(1H,d), 5.00(2H,s), 4.52(2H, s)

### Synthesis of 3-oxo-3,4-dihydro-2H-benzo[b][1,4]oxazine-8-sulfonyl chloride

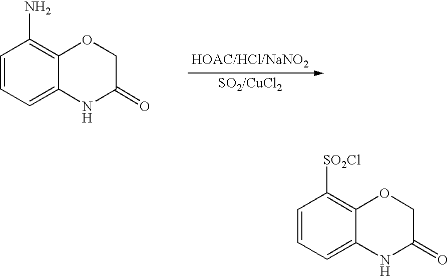

Into a 1000 mL 3-necked round-bottom flask, was placed a solution of 8-amino-2H-benzo[b][1,4]oxazin-3(4H)-one (8.3 g, 50.61 mmol, 1.00 equiv) in CH₃CN (350 mL). To the above was added acetic acid (41.85 g, 696.34 mmol, 13.76 equiv) dropwise with stirring, while cooling to a temperature of 0° C. To the above was added HCl (27.1 g, 267.29 mmol, 5.28 equiv, 36%) dropwise with stirring, while cooling to a temperature of 0° C. This was followed by the addition of a solution of NaNO₂ (4.24 g, 61.45 mmol, 1.20 equiv) in 120 (5 mL), which was added dropwise with stirring, while cooling to a temperature of 0° C. over a time period of 10 min. The resulting solution was allowed to react, with stirring, for 30 min while the temperature was maintained at 0° C. in a bath of H₂O/ice. Then to the mixture was bubbled with sulfur dioxide for two h while the temperature was maintained at 0° C. in a bath of H₂O/ice. To the above was added CuCl₂.2H₂O (8.7 g, 51.18 mmol, 1.00 equiv) in several batches. Then to the mixture was bubbled with sulfur dioxide for three h while the temperature was maintained at 0° C. in a bath of H₂O/ice. The reaction mixture was allowed to react, with stirring, overnight while maintaining at 0-10° C. The reaction was monitored by TLC (EtOAc:PE=1:1). The reaction mixture was then quenched by the adding 200 g of H₂O/ice. The resulting solution was extracted three times with 1000 mL of CH₂Cl₂ and the organic layers combined and dried over Na₂SO₄ and concentrated by evaporation under vacuum using a rotary evaporator. The residue was purified by eluting through a column with a 1:15-1:1 EtOAc/PE solvent system. This resulted in 2.1 g (16%) of 3-oxo-3,4-dihydro-2H-benzo[b][1,4]oxazine-8-sulfonyl chloride as a yellow solid.

LC-MS (ES, m/z): [M+H+C₅H₁₁N2-C]+ calcd for C₁₃H₁₇N₃O₄S 312, found 312

¹H NMR (DMSO, 300 MHz, δ) 4.50(2H,s), 6.85 (2H, m), 7.27 (1H, m), 10.67(1H,s).

### Example 25

### Preparation of 3-(Pyrrolidin-1-yl)benzene-1-sulfonyl Chloride

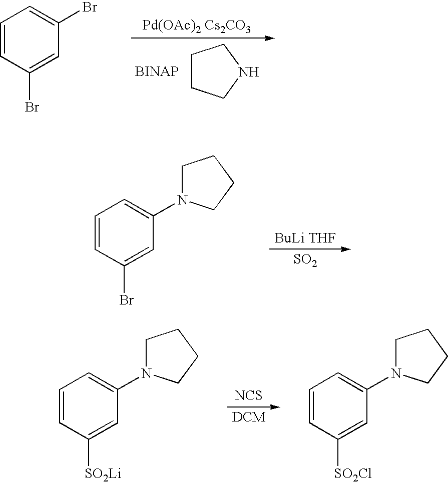

### Synthesis of 1-3-bromophenyl)pyrrolidine

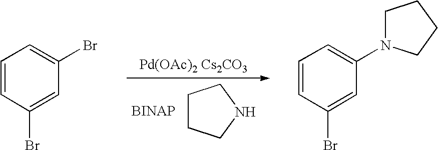

Into a 500 mL 3-necked round-bottom flask purged and maintained with an inert atmosphere of nitrogen, was placed a solution of 1,3-dibromobenzene (20 g, 84.78 mmol, 1.00 equiv) in toluene (300 mL). To this was added pyrrolidine (6.03 g, 84.80 mmol, 1.00 equiv). Addition of Pd(OAc)2 (190 mg, 0.85 mmol, 0.01 equiv) was next. This was followed by the addition of BINAP (760 mg, 2.53 mmol, 0.03 equiv). To the mixture was added Cs₂CO₃ (69.1 g, 211.96 mmol, 2.50 equiv). The resulting solution was allowed to react, with stirring, overnight while the temperature was maintained at 120° C. in a bath of oil. The reaction progress was monitored by TLC (EtOAc/PE=1:5). A filtration was performed. The filtrate was concentrated by evaporation under vacuum using a rotary evaporator. The residue was purified by eluting through a column with a PE solvent system. This resulted in 8.51 g (45%) of 1-(3-bromophenyl)pyrrolidine as a light yellow liquid.

LC-MS (ES, m/z): [M+H]+ calcd for C₁₀H₁₃BrN 226, found 226

### Synthesis of lithium 3-(pyrrolidin-1-yl)benzenesulfinate

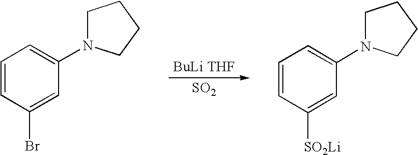

Into a 250 mL 3-necked round-bottom flask purged and maintained with an inert atmosphere of nitrogen, was placed a solution of 1-(3-bromophenyl)pyrrolidine (8.51 g, 37.64 mmol, 1.00 equiv) in TM (200 mL). To the above was added BuLi (18.07 mL, 45.18 mmol, 1.20 equiv, 2.5M) dropwise with stirring, while cooling to a temperature of −78° C. The resulting solution was allowed to react, with stirring, for 1 h while the temperature was maintained at −78° C. in a bath of N₂ (liquid). To the mixture was added SO₂ (4.82 g, 75.31 mmol, 2.00 equiv). The resulting solution was allowed to react, with stirring, for an additional 1 h while the temperature was maintained at room temperature. The reaction progress was monitored by TLC (EtOAc/PE=1:1). The resulting solution was diluted with 800 mL of n-hexane. The product was precipitated by the addition of collect the filter cake. This resulted in 8.2 g (100%) of lithium 3-(pyrrolidin-1-yl)benzenesulfinate as a orange solid.

### Synthesis of 3-(pyrrolidin-1-yl)benzene-1-sulfonyl chloride

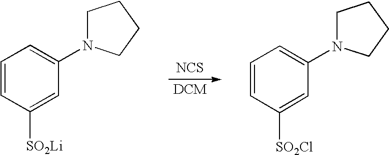

Into a 500 mL 3-necked round-bottom flask, was placed a solution of lithium 3-(pyrrolidin-1-yl)benzenesulfinate (8.18 g, 37.66 mmol, 1.00 equiv) in dichloromethane (300 mL). To the mixture was added NCS (6.03 g, 45.16 mmol, 1.20 equiv). The resulting solution was allowed to react, with stirring, for I h while the temperature was maintained at room temperature. The reaction progress was monitored by TLC (EtOAc/PE=1:1). The resulting mixture was washed one time with 100 mL of NaHSO₃ and two times with 200 mL of brine. The mixture was dried over MgSO₄ and concentrated by evaporation under vacuum using a rotary evaporator. This resulted in 7.2 g (75%) of 3-(pyrrolidin-1-yl)benzene-1-sulfonyl chloride as a yellow solid.

LC-MS (ES, m/z): [M+C₅H₁₁N₃—Cl+H]+ calcd for C₁₅H₂₄N₃O₂S 310, found 310

¹H NMR (CDCl₃, 300 MHz, δ): 2.06(4H, m), 3.33(4H, t), 6.81(1H, d), 7.06(1H,s), 7.25(1H,d), 7.37(1H, t)

### Example 26

### Receptor Activity

Assays for determining 5-HT₆ receptor activity, and selectivity of 5-HT₆ receptor activity are known within the art (see. e.g., Example 58 of U.S. Pat. No. 6,903,112).

The assay protocol for determining 5-HT₆ receptor activity generally entailed the incubation of membrane homogenates prepared from HeLa cells expressing the human 5-HT6 receptor with the radioligand ³H-lysergic acid diethylamide (³H-LSD) at a concentration of 1.29 nM. Concentrations ranging from 10⁻¹⁰ M to 10⁻⁵ M of test compound were incubated with the radioligand and the membrane homogenates. After 60 minutes incubation at 37° C. the reaction was terminated by vacuum filtration. The filters were washed with buffer and were counted for radioactivity using a liquid scintillation counter. The affinity of the test compound was calculated by determining the amount of the compound necessary to inhibit 50% of the binding of the radioligand to the receptor. Ki values were determined based upon the following equation:

Kᵢ=IC₅₀/(1+L/K_{D})

where L is the concentration of the radioligand used and K_{D} is the dissociation constant of the ligand for the receptor (both expressed in nM).

Preferred compounds of the invention show 5-HT6 binding activity with receptor Ki values of typically less than 100 nM, or preferably less than 1 nM. In addition, compounds of the invention show 5-HT6 functional activity with pA2 values of greater than 6 (IC₅₀ less than 1 μM).

In terms of selectivity, affinity for other serotonin receptors, specifically the 5-HT1A, 5-HT1B, 5-HT1D, 5-HT2A, 5-HT2B, 5-HT2C, 5-HT5A, and 5HT7 receptors, is expressed as the amount (in percent) of binding of the radioligand that is inhibited in the presence of 100 nM test compound. A lower percent inhibition indicates lower affinity for the serotonin receptor. Selected compounds show a percent inhibition of less than 50% for other serotonin receptors. In one embodiment, the compounds show a percent inhibition of less than 25% for other serotonin receptors

The preceding procedures and examples can be repeated with similar success by substituting the generically or specifically described reactants and/or operating conditions of this invention for those used in the preceding procedures and examples.

While the invention has been illustrated with respect to the production and of particular compounds, it is apparent that variations and modifications of the invention can be made without departing from the spirit or scope of the invention. Upon further study of the specification, further aspects, objects and advantages of this invention will become apparent to those skilled in the art.

We claim :

1. A compound of formula I: 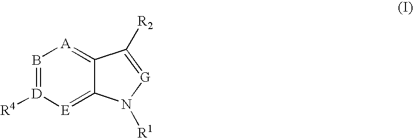
wherein
A, B, E, and G are each independently CH, CR³ or N;
D is C;
R¹ is SO₂Ar, wherein
Ar is selected from formulas (a)-(p): 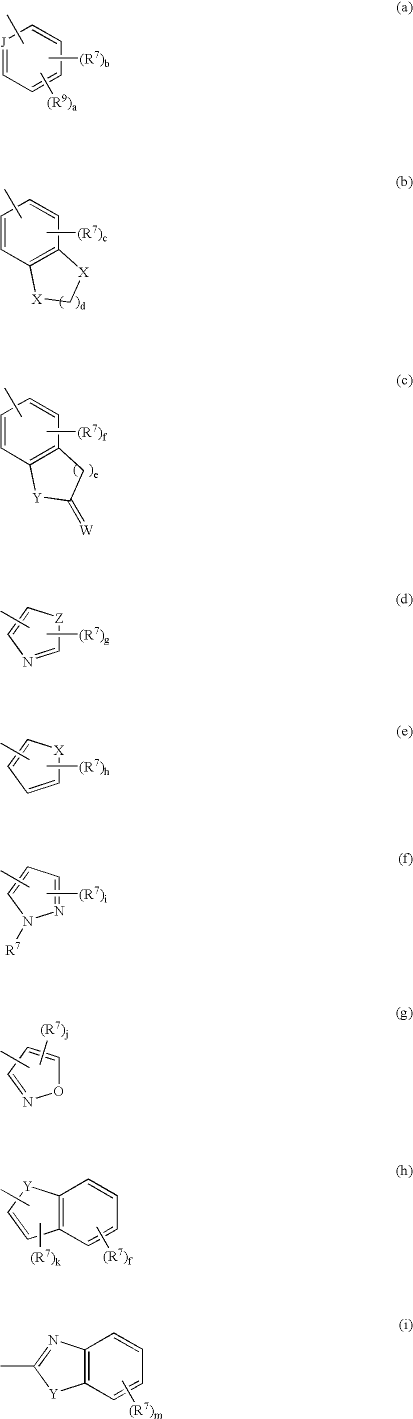 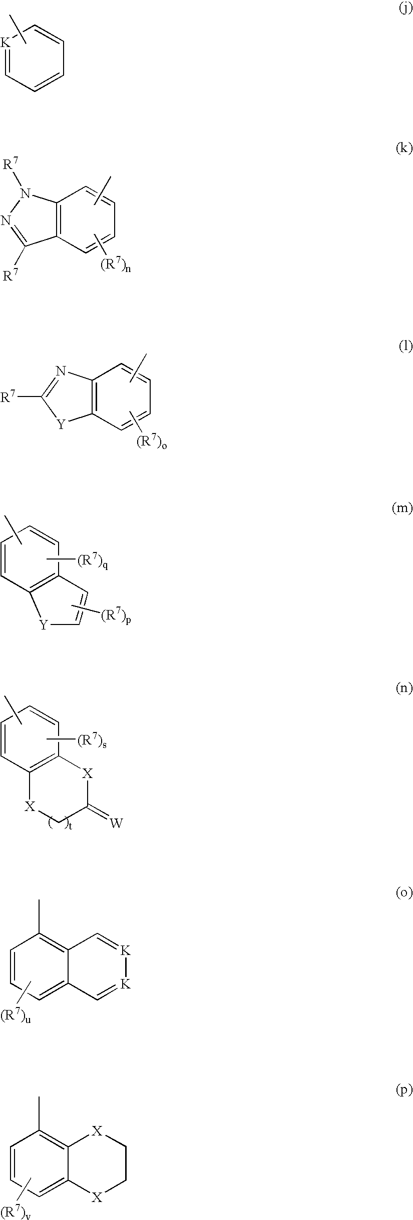
wherein
J is CR⁷ or N;
K is, in each instance is independently, CH or N;
W is O, S, or is absent;
X is, in each instance is independently, O or NR⁷;
Y is O, NR⁷ or S;
Z is S or NR⁷;
a is 1, 2, 3, 4 or 5;
b, l, and m are independently 0, 1, 2, 3 or 4;
c, f, h, n, o, q, s and u are independently 0, 1, 2 or 3;
d and e are independently 1, 2 or 3;
g, i, j, p, and u are independently 0, 1 or 2;
k and t are 0 or 1;
R² is H, C₁-C₆ alkyl, or COOR⁵
R³ is halogen, nitro,
alkyl having 1 to 8, preferably 1 to 4 carbon atoms, cycloalkyl having 3 to 12, preferably 3 to 8 carbon atoms, or cycloalkylalkyl having 4 to 12, preferably 4 to 8 carbon atoms, each of which is branched or unbranched and which is unsubstituted or substituted one or more times with halogen, C₁₋₄-alkyl, C₁₋₄-alkoxy, oxo, or any combination thereof, or
a heterocyclic group, which is saturated, partially saturated or unsaturated, having 5 to 10 ring atoms in which at least 1 ring atom is an N, O or S atom, which is unsubstituted or substituted one or more times by halogen, hydroxy, C₅₋₇-aryl, C₁₋₄-alkyl, C₁₋₄-alkoxy, cyano, halogenated C₁₋₄-alkyl, nitro, or any combination thereof,
R⁴ is 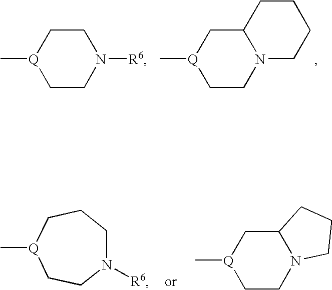
wherein each Q is independently N, CH, or C double bonded to an adjacent carbon,
R⁵ is H or alkyl having 1 to 8, preferably 1 to 4 carbon atoms,
R⁶ is H or alkyl having 1 to 8, preferably 1 to 4 carbon atoms, cycloalkyl having 3 to 12, preferably 3 to 8 carbon atoms, or cycloalkylalkyl having 4 to 12, preferably 4 to 8 carbon atoms, each of which is branched or unbranched and each of which is unsubstituted or substituted one or more times with halogen, C₁₋₄-alkyl, C₁₋₄-alkoxy, oxo, or any combination thereof;
R⁷ is, in each instance, independently
H, halogen, C(O)R⁸, CO₂R⁸, NR⁶COR⁸,
alkyl having 1 to 12, preferably 1 to 8 carbon atoms, which is branched or unbranched and which is unsubstituted or substituted one or more times by halogen, hydroxy, cyano, C₁₋₄-alkoxy, oxo or any combination thereof, and wherein optionally one or more —CH₂CH₂— groups is replaced in each case by —CH═CH— or —C≡C—,
alkoxy having 1 to 8, preferably 1 to 4 carbon atoms, which is branched or unbranched and which is unsubstituted or substituted one or more times by halogen,
cycloalkyl having 3 to 10, preferably 3 to 8 carbon atoms, which is unsubstituted or substituted one or more times by halogen, hydroxy, oxo, cyano, C₁₋₄-alkyl, C₁₋₄-alkoxy, or any combination thereof,
cycloalkylalkyl having 4 to 16, preferably 4 to 12 carbon atoms, which is unsubstituted or substituted in the cycloalkyl portion and/or the alkyl portion one or more times by halogen, oxo, cyano, hydroxy, C₁₋₄-alkyl, C₁₋₄-alkoxy or any combination thereof,
aryl having 6 to 14 carbon atoms, which is unsubstituted or substituted one or more times by halogen, CF₃, OCF₃, C₁₋₄-alkyl, hydroxy, C₁₋₄-alkoxy, nitro, methylenedioxy, ethylenedioxy, cyano, or any combination thereof,
arylalkyl in which the aryl portion has 6 to 14 carbon atoms and the alkyl portion, which is branched or unbranched, has 1 to 5 carbon atoms, wherein the arylalkyl radical is unsubstituted, substituted in the aryl portion one or more times by halogen, CF₃, OCF₃, C₁₋₄-alkyl, hydroxy, C₁₋₄-alkoxy, nitro, cyano, methylenedioxy, ethylenedioxy, or any combination thereof, and/or substituted in the alkyl portion one or more times by halogen, oxo, hydroxy, cyano, or any combination thereof, and wherein in the alkyl portion one or more —CH₂CH₂— groups are each optionally replaced by —CH═CH— or —C/C—, and one or more —CH₂— groups are each optionally replaced by —O— or —NH—,
a heterocyclic group, which is saturated, partially saturated or unsaturated, having 5 to 10 ring atoms in which at least 1 ring atom is an N, O or S atom, which is unsubstituted or substituted one or more times by halogen, hydroxy, C₅₋₇-aryl, C₁₋₄-alkyl, C₁₋₄-alkoxy, cyano, trifluoromethyl, nitro, oxo, or any combination thereof, or
a heterocycle-alkyl group, wherein the heterocyclic portion is saturated, partially saturated or unsaturated, and has 5 to 10 ring atoms in which at least 1 ring atom is an N, O or S atom, and the alkyl portion is branched or unbranched and has 1 to 5 carbon atoms, the heterocycle-alkyl group is unsubstituted,
substituted one or more times in the heterocyclic portion by halogen, OCF₃, hydroxy, C₅₋₇-aryl, C₁₋₄-alkyl, C₁₋₄-alkoxy, cyano, trifluoromethyl, nitro, oxo, or any combination thereof, and/or substituted in the alkyl portion one or more times by halogen, oxo, hydroxy, cyano, or any combination thereof, and wherein in the alkyl portion one or more —CH₂CH₂— groups are each optionally replaced by —CH═CH— or —C/C—, and one or more —CH₂— groups are each optionally replaced by —O— or —NH—;
R⁸ is in each instance, independently, H or alkyl having 1 to 8, carbon atoms, preferably 1 to 4 carbon atoms, which is branched or unbranched and which is unsubstituted or substituted one or more times by halogen;
R⁹ is an amino (NH₂): C₁₋₄-alkylamino, C₁₋₄-dialkylamino, NR¹⁰C(O)R¹⁰, cyano, methoxy, or heterocyclic group, which is saturated, partially saturated or unsaturated, having 5 to 10 ring atoms in which at least 1 ring atom is an N, O or S atom, which is unsubstituted or substituted one or more times by halogen, hydroxy, C₅₋₇-aryl, C₁₋₄-alkyl, C₁₋₄-alkoxy, cyano, halogenated C₁₋₄-alkyl, nitro, or any combination thereof, or —C(O)-heterocyclic group, and
R¹⁰ in each instance, is independently H or alkyl having 1 to 8, preferably 1 to 4 carbon atoms, which is branched or unbranched and which is unsubstituted or substituted one or more times with halogen, C₁₋₄-alkyl, C₁₋₄-alkoxy, oxo, or any combination thereof;
or a pharmaceutically acceptable salt or solvate thereof, or a solvate of pharmaceutically acceptable salt thereof;
with the following provisos:
(i) wherein if A, B, E, and G are CH or CR³ D is C, and Ar is (j) then R⁴ is not 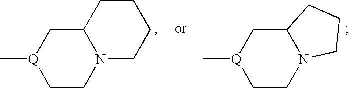
(ii) wherein if A, B, and E are CH or CR³, D is C, G is N, R² is H, and Ar is (j) wherein K is CH, or (h) wherein Y is S, then R⁴ is not

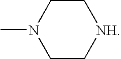

2. The compound of claim 1, wherein R² is H.

3. The compound of claim 1, wherein A and B are each CH.

4. The compound of claim 1, wherein R⁴ is

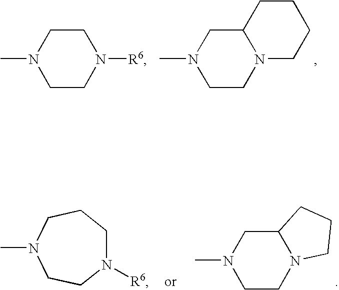

5. The compound of claim 4, wherein R⁶ is H, methyl or ethyl.

6. The compound of claim 1, wherein R⁷ is C₁₋₄-alkyl, halogenated C₁₋₄-alkyl, aryl, CO₂R₈, NR⁶COR₈, halogen, or C(O)R⁸.

7. The compound of claim 1, wherein Ar (a), (b), (c′), (j), (m), (n), or (p):

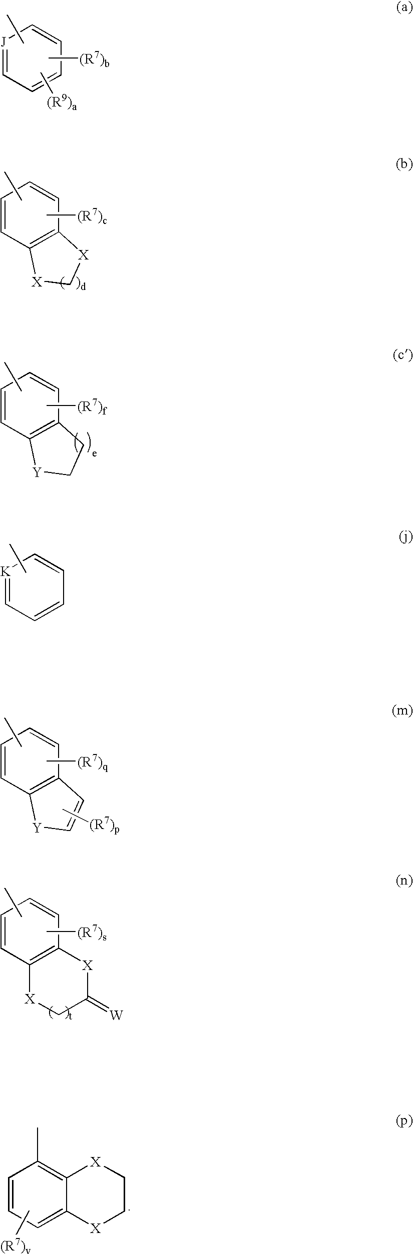

8. The compound of claim 7, wherein Ar is (b), d is 2, one X is O and the second X is NR7.

9. The compound of claim 7, wherein Ar is (c′) and e is 1.

10. The compound of claim 7, wherein Ar is (n), t is 1 and W is present.

11. The compound of claim 1, wherein A and B are CH, D is C, E and G are CH or N, and R¹ is SO₂Ar wherein Ar is phenyl substituted at least once by 3-methoxypyrrolidinyl, 3-hydroxypyrrolidinyl, or pyrrolidin-3-ol, or Ar is a substituted or unsubstituted aryl selected from pyrrolo[3-b]pyridinyl, benzofuranyl, dihydroindolyl, piperazinyl-indazolyl, and pyrazolo[3,4-b]pyridinyl.

12. The compound of claim 1, wherein A and B are CH, D is CR, and R¹ is SO₂Ar wherein Ar is an unsubstituted phenyl or unsubstituted pyridyl, and R⁴ is 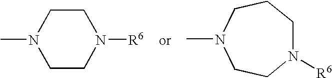
wherein R₆ is H or methyl.

13. The compound of claim 1, wherein at least one of A, B, and E is CR³ or N.

14. The compound of claim 1, wherein R⁴ is 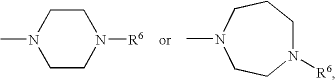
wherein R₆ is H or methyl.

15. The compound of claim 1, wherein G is CH or CR⁴.

16. The compound of claim 1, wherein the compound is a hydroformate salt.

17. A compound selected from the group consisting of:
1 4-methyl-7-[(6-piperazin-1-yl-1H-indol-1-yl)sulfonyl]-3,4-dihydro-2H-1,4-benzoxazine
2 1-methyl-5-[(6-piperazin-1-yl-1H-indol-1-yl)sulfonyl]-1H-indole
3 1-[(1-acetyl-2,3-dihydro-1H-indol-5-yl)sulfonyl]-6-piperazin-1-yl-1H-indole
4 1-{[3-(3-methoxypyrrolidin-1-yl)phenyl]sulfonyl}-6-(4-methylpiperazin-1-yl)-1H-pyrazolo[3,4-b]pyridine
5 1-{[3-(3-methoxypyrrolidin-1-yl)phenyl]sulfonyl}-6-piperazin-1-yl-1H-pyrazolo[3,4-b]pyridine
6 7-[(6-piperazin-1-yl-1H-pyrazolo[3,4-b]pyridin-1-yl)sulfonyl]-2H-1,4-benzoxazin-3(4H)-one
7 1-{[3-(3-methoxypyrrolidin-1-yl)phenyl]sulfonyl}-6-(4-methylpiperazin-1-yl)-1H-indazole
8 7-{[6-(4-methylpiperazin-1-yl)-1H-pyrazolo[3,4-b]pyridin-1-yl]sulfonyl}-2H-1,4-benzoxazin-3(4H)-one
9 7-{[6-(4-methylpiperazin-1-yl)-1H-indazol-1-yl]sulfonyl}-2H-1,4-benzoxazin-3(4H)-one
10 7-{[6-(4-methylpiperazin-1-yl)-1H-indol-1-yl]sulfonyl}-2H-1,4-benzoxazin-3(4H)-one
11 7-[(6-piperazin-1-yl-1H-indol-1-yl)sulfonyl]-2H-1,4-benzoxazin-3 (4H)-one
12 1-{[3-(3-methoxypyrrolidin-1-yl)phenyl]sulfonyl}-6-(4-methylpiperazin-1-yl)-1H-indole
13 1-{[3-(3-methoxypyrrolidin-1-yl)phenyl]sulfonyl}-6-piperazin-1-yl-1H-indole
14 1-[(1-acetyl-2,3-dihydro-1H-indol-5-yl)sulfonyl]-6-(4-methylpiperazin-1-yl)-1H-pyrrolo[2,3-b]pyridine
15 2-{1-[(1-acetyl-2,3-dihydro-1H-indol-5-yl)sulfonyl]-1H-indol-6-yl}octahydro-2H-pyrido[1,2-a]pyrazine
16 2-{1-[(1-acetyl-2,3-dihydro-1H-indol-5-yl)sulfonyl]-1H-indol-6-yl}octahydropyrrolo[1,2-a]pyrazine
17 1-{[3-(3-methoxypyrrolidin-1-yl)phenyl]sulfonyl}-6-(4-methylpiperazin-1H-pyrrolo[2,3-b]pyridine
18 2-(1-{[3-(3-methoxypyrrolidin-1-yl)phenyl]sulfonyl}-1H-indol-6-yl)octahydro-2H-pyrido[1,2-a]pyrazine
19 2-(1-{[3-(3-methoxypyrrolidin-1-yl)phenyl]sulfonyl}-1H-indol-6-yl)octahydropyrrolo[1,2-a]pyrazine
20 1-[(1-acetyl-2,3-dihydro-1H-indol-5-yl)sulfonyl]-6-(4-methylpiperazin-1-yl)-1H-pyrazolo[3,4-b]pyridine
21 1-[(1-acetyl-2,3-dihydro-1H-indol-5-yl)sulfonyl]-6-(4-methylpiperazin-1-yl)-1H-indazole
22 1-[(1-acetyl-2,3-dihydro-1H-indol-5-yl)sulfonyl]-6-(4-methylpiperazin-1-yl)-1H-indole
23 4-methyl-7-{[6-(4-methylpiperazin-1-yl)-1H-indazol-1-yl]sulfonyl}-3,4-dihydro-2H-1,4-benzoxazine
24 4-methyl-7-{[6-(4-methylpiperazin-1-yl)-1H-pyrazolo[3,4-b]pyridin-1-yl]sulfonyl}-3,4-dihydro-2H-1,4-benzoxazine
25 4-methyl-7-{[6-(4-methylpiperazin-1-yl)-1H-pyrrolo[2,3-b]pyridin-1-yl]sulfonyl}-3,4-dihydro-2H-1,4-benzoxazine
26 4-methyl-7-{[6-(octahydro-2H-pyrido[1,2-a]pyrazin-2-yl)-1H-indol-1-yl]sulfonyl}-3,4-dihydro-2H-1,4-benzoxazine
27 7-{[6-(hexahydropyrrolo[1,2-a]pyrazin-2(1H)-yl)-1H-indol-1-yl]sulfonyl}-3,4-dihydro-2H-1,4-benzoxazine
28 7-{[6-(4-methylpiperazin-1-yl)-1H-pyrrolo[2,3-b]pyridin-1-yl]sulfonyl}-2H-1,4-benzoxazin-3(4H)-one
29 7-{[6-(octahydro-2H-pyrido[1,2-a]pyrazin-2-yl)-1H-indol-1-yl]sulfonyl}-2H-1,4-benzoxazin-3(4H)-one
30 7-{[6-(hexahydropyrrolo[1,2-a]pyrazin-2(1H)-yl)-1H-indol-1-yl]sulfonyl}-2H-1,4-benzoxazin-3(4H)-one
31 1-(1-benzofuran-5-ylsulfonyl)-6-(4-methylpiperazin-1-yl)-1H-pyrrolo[2,3-b]pyridine
32 2-[1-(1-benzofuran-5-ylsulfonyl)-1H-indol-6-yl]octahydro-2H-pyrido[1,2-a]pyrazine
33 1-[(1-acetyl-2,3-dihydro-1H-indol-5-yl)sulfonyl]-6-(1,4-diazepan-1-yl)-1H-indazole
34 7-{[6-(1,4-diazepan-1-yl)-1H-indazol-1-yl]sulfonyl}-4-methyl-3,4-dihydro-2H-1,4-benzoxazine
35 1-(1-benzofuran-6-ylsulfonyl)-6-(1,4-diazepan-1-yl)-1H-indazole
36 1-(1-benzofuran-5-ylsulfonyl)-6-(1,4-diazepan-1-yl)-1H-indazole
37 1-[(1-acetyl-2,3-dihydro-1H-indol-5-yl)sulfonyl]-6-(4-methyl-1,4-diazepan-1-yl)-1H-indole
38 4-methyl-7-{[6-(4-methyl-1,4-diazepan-1-yl)-1H-indol-1-yl]sulfonyl}-3,4-dihydro-2H-1,4-benzoxazine
39 1-{[3-(3-methoxypyrrolidin-1-yl)phenyl]sulfonyl}-6-(4-methyl-1,4-diazepan-1-yl)-1H-indole
40 1-[(1-acetyl-2,3-dihydro-1H-indol-5-yl)sulfonyl]-6-(1,4-diazepan-1-yl)-1H-indole
41 7-{[6-(1,4-diazepan-1-yl)-1H-indol-1-yl]sulfonyl}-4-methyl-3,4-dihydro-2H-1,4-benzoxazine
42 6-(1,4-diazepan-1-yl)-1-{[3-(3-methoxypyrrolidin-1-yl)phenyl]sulfonyl}-1H-indole
43 1-(1-benzofuran-6-ylsulfonyl)-6-(1,4-diazepan-1-yl)-1H-indole
44 1-(1-benzofuran-5-ylsulfonyl)-6-(1,4-diazepan-1-yl)-1H-indole
45 6-(1,4-diazepan-1-yl)-1-{[3-(3-methoxypyrrolidin-1-yl)phenyl]sulfonyl}-1H-indazole
46 4-methyl-7-{[6-(4-methyl-1,4-diazepan-1-yl)-1H-indazol-1-yl]sulfonyl}-3,4-dihydro-2H-1,4-benzoxazine
47 1-{[3-(3-methoxypyrrolidin-1-yl)phenyl]sulfonyl}-6-(4-methyl-1,4-diazepan-1-yl)-1H-indazole
48 1-(1-benzofuran-6-ylsulfonyl)-6-(4-methyl-1,4-diazepan-1-yl)-1H-indazole
49 1-(1-benzofuran-5-ylsulfonyl)-6-(4-methyl-1,4-diazepan-1-yl)-1H-indazole
50 1-[(1-acetyl-2,3-dihydro-1H-indol-5-yl)sulfonyl]-6-piperazin-1-yl-1H-indole
51 4-methyl-7-{[6-(4-methylpiperazin-1-yl)-1H-indazol-1-yl]sulfonyl}-3,4-dihydro-2H-1,4-benzoxazine
52 ethyl 1-[(4-methyl-3,4-dihydro-2H-1,4-benzoxazin-7-yl)sulfonyl]-6-(4-methylpiperazin-1-yl)-1H-indazole-3-carboxylate
53 ethyl 1-{[3-(3-hydroxypyrrolidin-1-yl)phenyl]sulfonyl}-6-(4-methylpiperazin-1-yl)-1H-indazole-3-carboxylate
54 ethyl 1-[(4-methyl-3,4-dihydro-2H-1,4-benzoxazin-7-yl)sulfonyl]-6-piperazin-1-yl-1H-indazole-3-carboxylate
55 ethyl 1-{[3-(3-methoxypyrrolidin-1-yl)phenyl]sulfonyl}-6-piperazin-1-yl-1-H-indazole-3-carboxylate
56 7-{[3-ethyl-6-(4-methylpiperazin-1-yl)-1H-indazol-1-yl]sulfonyl}-4-methyl-3,4-dihydro-2H-1,4-benzoxazine
57 3-ethyl-1-{[3-(3-methoxypyrrolidin-1-yl)phenyl]sulfonyl}-6-(4-methylpiperazin-1-yl)-1H-indazole
58 1-(3-{[3-ethyl-6-(4-methylpiperazin-1-yl)-1H-indazol-1-yl]sulfonyl}phenyl)pyrrolidin-3-ol
59 7-[(3-ethyl-6-piperazin-1-yl-1H-indazol-1-yl)sulfonyl]-4-methyl-3,4-dihydro-2H-1,4-benzoxazine
60 3-ethyl-1-{[3-(3-methoxypyrrolidin-1-yl)phenyl]sulfonyl}-6-piperazin-1-yl-1H-indazole
61 1-{3-[(3-ethyl-6-piperazin-1-yl-1H-indazol-1-yl)sulfonyl]phenyl}pyrrolidin-3-ol
62 1-[(1-acetyl-2,3-dihydro-1H-indol-5-yl)sulfonyl]-3-ethyl-6-piperazin-1-yl-1H-indazole
63 ethyl 1-{[3-(3-methoxypyrrolidin-1-yl)phenyl]sulfonyl}-6-(4-methylpiperazin-1-yl)-1H-indazole-3-carboxylate
64 7-[(3-ethyl-6-piperazin-1-yl-1H-indazol-1-yl)sulfonyl]-2H-1,4-benzoxazin-3(4H)-one
65 7-[(6-piperazin-1-yl-1H-indol-1-yl)sulfonyl]-2H-1,4-benzoxazin-3(4H)-one
66 6-[(3-ethyl-6-piperazin-1-yl-1H-indol-1-yl)sulfonyl]-4-methyl-3,4-dihydro-2H-1,4-benzoxazine
67 6-[(3-ethyl-6-piperazin-1-yl-1H-indol-1-yl)sulfonyl]-2H-1,4-benzoxazin-3(4)-one
68 3-Ethyl-1-[3-((S)-3-methoxy-pyrrolidin-1-yl)-benzenesulfonyl]-6-piperazin-1-yl-1H-indole
69 (S)-1-[3-(3-Ethyl-6-piperazin-1-yl-indole-1-sulfonyl)-phenyl]-pyrrolidin-3-ol
70 5-(6-Piperazin-1-yl-indole-1-sulfonyl)-4H-benzo[1,4]oxazin-3-one
71 4-methyl-7-[(6-piperazin-1-yl-1H-indazol-1-yl)sulfonyl]-3,4-dihydro-2H-1,4-benzoxazine
72 4-methyl-7-[(3-methyl-6-piperazin-1-yl-1H-indazol-1-yl)sulfonyl]-3,4-dihydro-2H-1,4-benzoxazine
73 4-methyl-7-[(3-methyl-6-piperazin-1-yl-1H-indol-1-yl)sulfonyl]-3,4-dihydro-2H-1,4-benzoxazine
74 7-[(3-ethyl-6-piperazin-1-yl-1H-indol-1-yl)sulfonyl]-4-methyl-3,4-dihydro-2H-1,4-benzoxazine
75 4-methyl-6-[(6-piperazin-1-yl-1H-indazol-1-yl)sulfonyl]-3,4-dihydro-2H-1,4-benzoxazine
76 4-methyl-6-[(6-piperazin-1-yl-1H-indol-1-yl)sulfonyl]-3,4-dihydro-2H-1,4-benzoxazine
77 4-Methyl-6-(3-methyl-6-piperazin-1-yl-indazole-1-sulfonyl)-3,4-dihydro-2H-benzo[1,4]oxazine
78 3-Ethyl-1-[3-((S)-3-methoxy-pyrrolidin-1-yl)-benzenesulfonyl]-6-piperazin-1-yl-1H-indazole
79 3-Ethyl-1-[3-((S)-3-methoxy-pyrrolidin-1-yl)-benzenesulfonyl]-6-piperazin-1-yl-1H-indazole
80 1-[3-((S)-3-Methoxy-pyrrolidin-1-yl)-benzenesulfonyl]-3-methyl-6-piperazin-1-yl-1H-indazole
81 1-[3-((S)-3-Methoxy-pyrrolidin-1-yl)-benzenesulfonyl]-3-methyl-6-piperazin-1-yl-1H-indole
82 4-Methyl-6-(3-methyl-6-piperazin-1-yl-indole-1-sulfonyl)-3,4-dihydro-2H-benzo[1,4]oxazine
83 6-(3-Ethyl-6-piperazin-1-yl-indole-1-sulfonyl)-4-methyl-3,4-dihydro-2H-benzo[1,4]oxazine
84 7-(3-Ethyl-6-piperazin-1-yl-indazole-1-sulfonyl)-4-methyl-3,4-dihydro-2H-benzo[1,4]oxazine
85 6-(6-Piperazin-1-yl-indazole-1-sulfonyl)-4H-benzo[1,4]oxazin-3-one
86 6-(6-Piperazin-1-yl-indole-1-sulfonyl)-4H-benzo[1,4]oxazin-3-one
87 6-(3-Methyl-6-piperazin-1-yl-indazole-1-sulfonyl)-4H-benzo[1,4]oxazin-3-one
88 6-(3-Methyl-6-piperazin-1-yl-indole-1-sulfonyl)-4H-benzo[1,4]oxazin-3-one
89 4-methyl-7-[(6-piperazin-1-yl-1H-indazol-1-yl)sulfonyl]-3,4-dihydro-2H-pyrido[3,2-b][1,4]oxazine
90 1-(2,3-dihydro-1,4-benzodioxin-6-ylsulfonyl)-6-piperazin-1-yl-1H-indole
91 {3-[(6-piperazin-1-yl-1H-indazol-1-yl)sulfonyl]phenyl}(pyridin-2-yl)methanone
92 1-(2,3-dihydro-1,4-benzodioxin-6-ylsulfonyl)-6-piperazin-1-yl-1H-indazole
93 {3-[(6-piperazin-1-yl-1H-indol-1-yl)sulfonyl]phenyl}(pyridin-2-yl)methanone
94 3-[(6-piperazin-1-yl-1H-indazol-1-yl)sulfonyl]benzonitrile
95 3-[(6-piperazin-1-yl-1H-indol-1-yl)sulfonyl]benzonitrile
96 6-(4-methylpiperazin-1-yl)-1-(phenylsulfonyl)-1H-indole
97 1-(phenylsulfonyl)-6-piperazin-1-yl-1H-indole
98 ethyl 6-(4-methylpiperazin-1-yl)-1-(phenylsulfonyl)-1H-indazole-3-carboxylate
99 6-(4-methylpiperazin-1-yl)-1-(phenylsulfonyl)-1H-indazole-3-carboxylic acid
100 ethyl 1-(phenylsulfonyl)-6-piperazin-1-yl-1H-indazole-3-carboxylate
101 3-ethyl-6-(4-methylpiperazin-1-yl)-1-(phenylsulfonyl)-1H-indazole
102 3-ethyl-1-(phenylsulfonyl)-6-piperazin-1-yl-1H-indazole
103 ethyl 6-(4-methylpiperazin-1-yl)-1-(phenylsulfonyl)-1H-indazole-3-carboxylate
104 6-piperazin-1-yl-1-(pyridin-3-ylsulfonyl)-1H-indole
105 6-(4-methylpiperazin-1-yl)-1-(pyridin-3-ylsulfonyl)-1H-indole
106 6-(4-methylpiperazin-1-yl)-1-(pyridin-3-ylsulfonyl)-1H-indazole
107 6-piperazin-1-yl-1-(pyridin-3-ylsulfonyl)-1H-indole
108 6-(4-methylpiperazin-1-yl)-1-(pyridin-3-ylsulfonyl)-1H-pyrrolo[2,3-b]pyridine
109 6-(4-methyl-1,4-diazepan-1-yl)-1-(pyridin-3-ylsulfonyl)-1H-indole
110 6-(1,4-diazepan-1-yl)-1-(pyridin-3-ylsulfonyl)-1H-indole
111 6-(1,4-diazepan-1-yl)-1-(pyridin-3-ylsulfonyl)-1H-indazole
112 ethyl 6-piperazin-1-yl-1-(pyridin-3-ylsulfonyl)-1H-indazole-3-carboxylate
113 3-ethyl-6-(4-methylpiperazin-1-yl}-1-(pyridin-3-ylsulfonyl)-1H-indazole
114 3-ethyl-6-piperazin-1-yl-1-(pyridin-3-ylsulfonyl)-1H-indazole
115 6-piperazin-1-yl-1-(pyridin-3-ylsulfonyl)-1H-indole
116 1-[(3-fluorophenyl)sulfonyl]-6-piperazin-1-yl-1H-indole
117 1-[(2-fluorophenyl)sulfonyl]-6-piperazin-1-yl-1H-indole
118 1-[(2,4-difluorophenyl)sulfonyl]-6-piperazin-1-yl-1H-indole
119 1-[(2,5-difluorophenyl)sulfonyl]-6-piperazin-1-yl-1H-indole
120 1-[(3-chlorophenyl)sulfonyl]-6-piperazin-1-yl-1H-indole
121 1-[(2-chlorophenyl)sulfonyl]-6-piperazin-1-yl-1H-indole
122 1-[(3-fluorophenyl)sulfonyl]-6-piperazin-1-yl-1H-indazole
123 1-[(2-fluorophenyl)sulfonyl]-6-piperazin-1-yl-1H-indazole
124 1-[(2,4-difluorophenyl)sulfonyl]-6-piperazin-1-yl-1H-indazole
125 1-[(2,5-difluorophenyl)sulfonyl]-6-piperazin-1-yl-1H-indazole
126 1-[(3-chlorophenyl)sulfonyl]-6-piperazin-1-yl-1H-indazole
127 1-[(2-chlorophenyl)sulfonyl]-6-piperazin-1-yl-1H-indazole
128 1-[(3-methoxyphenyl)sulfonyl]-6-piperazin-1-yl-1H-indole
129 1-[(2-methoxyphenyl)sulfonyl]-6-piperazin-1-yl-1H-indole
130 -[(4-methoxyphenyl)sulfonyl]-6-piperazin-1-yl-1H-indole
131 1-[(3,4-dimethoxyphenyl)sulfonyl]-6-piperazin-1-yl-1H-indole
132 1-[(2,5-dimethoxyphenyl)sulfonyl]-6-piperazin-1-yl-1H-indole
133 1-[(3-methoxyphenyl)sulfonyl]-6-piperazin-1-yl-1H-indazole
134 1-[(2-methoxyphenyl)sulfonyl]-6-piperazin-1-yl-1H-indazole
135 1-[(4-methoxyphenyl)sulfonyl]-6-piperazin-1-yl-1H-indazole
136 1-[(3,4-dimethoxyphenyl)sulfonyl]-6-piperazin-1-yl-1H-indazole
137 1-[(2,5-dimethoxyphenyl)sulfonyl]-6-piperazin-1-yl-1H-indazole
138 1-(1-naphthylsulfonyl)-6-piperazin-1-yl-1H-indazole
139 1-(1-naphtbylsulfonyl)-6-piperazin-1-yl-1H-indole
140 3-[(6-piperazin-1-yl-1H-indol-1-yl)sulfonyl]quinoline
141 3-[(6-piperazin-1-yl-1H-indazol-1-yl)sulfonyl]quinoline
or a free base, a pharmaceutically acceptable salt, or solvate thereof.

18. A method of treating a central nervous system disorder (CNS), a memory/cognitive impairment, a gastrointestinal (GI) disorder, or a polyglutamine-repeat disease comprising administering a pharmacologically effective amount of a compound according to claim 1 to a patient in need thereof.

19. The method of claim 18 wherein the CNS disorder is Alzheimers disease, Parkinson's disease, Huntington's disease, anxiety, depression, manic depression, epilepsy, obsessive compulsive disorders, migraine, sleep disorders, feeding disorders such as anorexia and bulimia, panic attacks, attention deficit hyperactivity disorder (ADHD), attention deficit disorder (ADD), withdrawal from drug abuse, psychoses, or disorders associated with spinal trauma and/or head injury.

20. The method of claim 18, wherein the memory/cognitive impairment is associated with Alzheimer's disease, schizophrenia, Parkinson's disease, Huntington's disease Pick's disease, Creutzfeld Jakob disease, HM, cardiovascular disease, head trauma or age-related cognitive decline.

21. The method of claim 18, wherein the GI disorder is functional bowel disorder, constipation, gastroesophageal reflux disease (GERD), nocturnal-GERD, irritable bowel syndrome (IBS), constipation-predominant IBS (IBS-c) or alternating constipation/diarrhea IBS.

22. The method of claim 18, wherein the compound of claim 1 is administered in a pharmaceutically acceptable carrier.

23. A method of modulating 5-HT6 receptor activity comprising administering a pharmacologically effective amount of a compound according to claim 1 to a patient in need thereof.

24. The method of claim 23, wherein the compound of claim 1 is administered in a pharmaceutically acceptable carrier.

25. A pharmaceutical composition comprising a therapeutically effective amount of the compound of claim 1 and a pharmaceutically acceptable carrier.
